(19) United States
(12) Patent Application Publication
(10) Pub. No.: US 2024/0165151 A1
(43) Pub. Date: May 23, 2024

(54) COMPOSITIONS AND METHODS FOR TREATING UPPER RESPIRATORY INFECTIONS

(71) Applicant: SaNOtize Research and Development Corp., Vancouver (CA)

(72) Inventors: Gilly Regev, Vancouver (CA); Christopher C. Miller, North Vancouver (CA)

(73) Assignee: SaNOtize Research and Development Corp., Vancouver, BC (CA)

(21) Appl. No.: 17/920,984

(22) PCT Filed: Apr. 22, 2021
(86) PCT No.: PCT/IB2021/000268

§ 371 Date: Oct. 24, 2022

Related U.S. Application Data
(60) Provisional application No. 63/014,117, filed on Apr. 22, 2020.
(60) Provisional application No. 63/160,627, filed on Mar. 12, 2021.

(51) Int. Cl. A61K 33/00; A61K 9/00; A61K 9/08; A61P 31/14

(52) U.S. Cl. CPC A61K 33/00; A61K 9/0078; A61K 9/08; A61P 31/14

(57) ABSTRACT

The present disclosure is drawn to methods and compositions for treating infections and minimizing transmissibility. The method can comprise treating a subject for infection with Severe Acute Respiratory Syndrome Coronavirus (SARSr-CoV) comprising administering a therapeutically effective amount of a nitric oxide releasing solution (NORS) to the subject. The NORS can be administered as a spray having an average droplet volume which contains treatment within the upper respiratory tract. The method of minimizing subject-to-subject transmissibility for a pathogen can comprise administering a therapeutically effective amount of a nitric oxide releasing solution (NORS) to the subject. NORS can include at least one nitric oxide releasing compound and acidifying agent, which can release a therapeutically effective amount of spray having an average droplet volume which contains treatment within the upper respiratory tract.

### PRIORITY DATA

This application claims the benefit of U.S. Provisional Application Ser. No. 63/014,117, filed Apr. 22, 2020 and U.S. Provisional Application Ser. No. 63/160,627, filed Mar. 12, 2021, which are each incorporated herein by reference in their entirety.

### FIELD OF THE DISCLOSURE

The present disclosure relates to methods of treating a subject with an infection. Accordingly, this invention involves the fields of chemistry, pharmaceutical sciences, medicine, and other health sciences.

### BACKGROUND

Severe Acute Respiratory Syndrome Coronavirus 2 (SARS-CoV-2) is a strain of Severe Acute Respiratory Syndrome Coronavirus (SARSr-CoV). SARS-CoV-2 is a single-stranded RNA virus that causes coronavirus disease 2019 (COVID-19). This virus is highly contagious in humans and causes, among other symptoms, respiratory illness that can ultimately result in death. The virus is primarily spread between people through close contact and via respiratory droplets produced from coughs or sneezes. People may also become infected by touching a contaminated surface and then touching their eyes, nose, or mouth. The virus mainly enters human cells by binding to the receptor angiotensin converting enzyme 2 (ACE2). New mechanisms of preventing and treating the COVID-19 condition and other respiratory conditions are being sought.

Many chemical compounds show promise for various applications or uses, but in remain unusable due to various challenges such as instability, difficulty in transport and administration, or for other reasons. One example of such a compound is nitric oxide (NO). Because NO is a free radical, it is highly reactive and presents a significant challenge to store and administer for therapeutic purposes.

### BRIEF DESCRIPTION OF THE DRAWINGS

Features and advantages of the disclosure will be apparent from the detailed description which follows, taken in conjunction with the accompanying drawings, which together illustrate, by way of example, features of the disclosure; and, wherein:

FIG. 1 illustrates the difference in the change from baseline in SARS-CoV-2 RNA between the active nitric oxide nasal spray (NONS) group and the placebo (saline) group from day 1 to day 6 in accordance with an example;

FIG. 2 depicts a total of 80 subjects (40 NONS, 40 Placebo) randomized into the trial and receiving study treatments in accordance with an example;

FIG. 3 shows a graphical representation of the (log) viral load change from baseline to Days 2, 4, and 6 in accordance with an example;

FIG. 4 displays a Kaplan Meier curve for the time-to SARS-CoV-2 (log) viral load reduction for threshold 3 achievement at day 2, day 4 and day 6 (ITT Population) in accordance with an example;

FIG. 5a shows the droplet size distribution for samples 1 to 3 in accordance with an example;

FIG. 5b shows the droplet size distribution for samples 4 to 6 in accordance with an example;

FIG. 5c shows the droplet size distribution for samples 1 and 4 in accordance with an example;

FIG. 5d shows the droplet size distribution for sample 1 in accordance with an example;

FIG. 5e shows the droplet size distribution for sample 4 in accordance with an example;

FIG. 6a depicts a study dosage regimen in accordance with an example;

FIG. 6b shows a change in total modified Lund Kennedy (MLK) endoscopic score pre and post treatment in accordance with an example; and

FIG. 6c shows a change in disease-specific quality of life Sino-Nasal Outcome Test (SNOT-22) pre and post treatment with NOSi in accordance with an example.

Reference will now be made to the exemplary embodiments illustrated, and specific language will be used herein to describe the same. It will nevertheless be understood that no limitation of the scope of the invention is thereby intended.

### DESCRIPTION OF EMBODIMENTS

Although the following detailed description contains many specifics for the purpose of illustration, a person of ordinary skill in the art will appreciate that many variations and alterations to the following details can be made and are considered to be included herein. As such, the following embodiments are set forth without any loss of generality to, and without imposing limitations upon, any claims set forth. It is also to be understood that the terminology used herein is for the purpose of describing particular embodiments only, and is not intended to be limiting. Unless defined otherwise, all technical and scientific terms used have the same meaning as commonly understood by one of ordinary skill in the art to which this disclosure belongs.

### Definitions

In this written description, the singular forms “a,” “an” and “the” provide express support for plural referents unless the context clearly dictates otherwise. Thus, for example, reference to “a particle” includes a plurality of particles.

In this application, “comprises,” “comprising,” “containing” and “having” and the like can have the meaning ascribed to them in U.S. Patent law and can mean “includes,” “including,” and the like, and are generally interpreted to be open ended terms. The terms “consisting of” or “consists of” are closed terms, and include only the components, structures, steps, or the like specifically listed in conjunction with such terms, as well as that which is in accordance with U.S. Patent law. “Consisting essentially of” or “consists essentially of” have the meaning generally ascribed to them by U.S. Patent law. In particular, such terms are generally closed terms, with the exception of allowing inclusion of additional items, materials, components, steps, or elements, that do not materially affect the basic and novel characteristics or function of the item(s) used in connection therewith. For example, trace elements present in a composition, but not affecting the compositions nature or characteristics would be permissible if present under the “consisting essentially of” language, even though not expressly recited in a list of items following such terminology. When using an open ended term, like “comprising” or “including,” in this written description it is understood that direct support should be afforded also to “consisting essentially of” language as well as “consisting of” language as if stated explicitly and vice versa.

The terms “first,” “second,” “third,” “fourth,” and the like in the description and in the claims, if any, are used for distinguishing between similar elements and not necessarily for describing a particular sequential or chronological order. It is to be understood that any terms so used are interchangeable under appropriate circumstances such that the embodiments described herein are, for example, capable of operation in sequences other than those illustrated or otherwise described herein. Similarly, if a method is described as comprising a series of steps, the order of such steps as presented is not necessarily the only order in which such steps may be performed, and certain of the stated steps may possibly be omitted and/or certain other steps not described herein may possibly be added to the method.

Occurrences of the phrase “in one embodiment,” or “in one aspect,” herein do not necessarily all refer to the same embodiment or aspect.

As used herein, “subject” refers to a mammal that may benefit from the administration of a nitric oxide releasing solution (NORS). In some embodiments the NORS can be administered as a nitric oxide nasal spray (NONS). In one aspect the mammal may be a human.

As used herein, the terms “treat,” “treatment,” or “treating” when used in conjunction with the administration of NORS, including when administered as a NONS, including compositions and dosage forms thereof, refers to administration to subjects who are either asymptomatic or symptomatic. In other words, “treat,” “treatment,” or “treating” can be to reduce, ameliorate or eliminate symptoms associated with a condition present in a subject, or can be prophylactic, (i.e. to prevent or reduce the occurrence of the symptoms in a subject). Such prophylactic treatment can also be referred to as prevention of the condition.

As used herein, the terms “formulation” and “composition” are used interchangeably and refer to a mixture of two or more compounds, elements, or molecules. In some aspects the terms “formulation” and “composition” may be used to refer to a mixture of one or more active agents with a carrier or other excipients. Compositions can take nearly any physical state, including solid, liquid (i.e. solution), or gas. Furthermore, the term “dosage form” can include one or more formulation(s) or composition(s) provided in a format for administration to a subject. In one example, a composition can be a solution that releases nitric oxide.

A “kit” can mean a package or container that includes a composition or dosage form along with instructions regarding application or administration of the composition or dosage form according to a given regimen or within specified time and amount parameters to treat one or more specific indications. For example, a kit could include a NORS composition in a specific volume or amount along with a set of instructions on appropriate administration of the NORS to a subject in order to treat a given condition (e.g., indication). Instructions may include direction for a single type of administration or indication, or for multiple types of administration or indications. Additionally, the amount and form of the NORS composition in the kit can be suitable for a single administration for treatment of a single indication, multiple administrations creating a regimen for one indication, or single or multiple administrations for multiple indications. For example, a composition or dosage form of a NORS composition can be provided as a NONS in the kit along with instructions for applying the dosage form in terms of amount and volume that is suitable to treat a plurality of indications, such as improving immunity in the sinus and throat areas against a viral or bacterial infection, or infections therewith, or associated symptoms.

As used herein “NORS” refers to a nitric oxide (NO) releasing solution, composition or substance. In one aspect, NO released from NORS may be a gas. Additionally, “NONS” refers to a NORS in the form of a nasal spray (e.g. a nitric oxide nasal spray).

As used herein a “therapeutic agent” refers to an agent that can have a beneficial or positive effect on a subject when administered to the subject in an appropriate or effective amount. In one aspect, NO can be a therapeutic agent. In another aspect, therapeutic agents can include non-NORS agents with physiologic activity, such as antibiotics, antihistamines, antivirals, antimicrobials, biological molecules, such as siRNA, cDNA, steroids, vasodilators, vasoconstrictors, analgesics, anti-inflammatories, etc. In some aspects, therapeutic agent can be used interchangeably with “active agent” or “drug”.

As used herein, an “effective amount” of an agent is an amount sufficient to accomplish a specified task or function desired of the agent. A “therapeutically effective amount” of a composition, drug, or agent refers to a non-toxic, but sufficient amount of the composition, drug, or agent, to achieve therapeutic results in treating or preventing a condition for which the composition, drug, or agent is known to be effective. It is understood that various biological factors may affect the ability of a substance to perform its intended task. Therefore, an “effective amount” or a “therapeutically effective amount” may be dependent in some instances on such biological factors. Further, while the achievement of therapeutic effects may be measured by a physician, veterinarian, or other qualified medical personnel using evaluations known in the art, it is recognized that individual variation and response to treatments may make the achievement of therapeutic effects a somewhat subjective decision. The determination of an effective amount or therapeutically effective amount is well within the ordinary skill in the art of pharmaceutical sciences and medicine. See, for example, Meiner and Tonascia, “Clinical Trials: Design, Conduct, and Analysis,” Monographs in Epidemiology and Biostatistics, Vol. 8 (1986).

As used herein, a “dosing regimen” or “regimen” such as “treatment dosing regimen,” or a “prophylactic dosing regimen” refers to how, when, how much, and for how long a dose of a composition can or should be administered to a subject in order to achieve an intended treatment or effect.

As used herein, “daily dose” refers to the amount of active agent administered to a subject over a 24-hour period of time. The daily dose can be administered in one or more administrations during the 24-hour period. In one embodiment, the daily dose provides for 2-6 administrations in a 24-hour period.

As used herein, an “acute” condition refers to a condition that can develop rapidly and have distinct symptoms needing urgent or semi-urgent care. By contrast, a “chronic” condition refers to a condition that is typically slower to develop and lingers or otherwise progresses over time. Some examples of acute conditions can include without limitation, an asthma attack, bronchitis, a heart attack, pneumonia, and the like. Some examples of chronic conditions can include without limitation, arthritis, diabetes, hypertension, high cholesterol, and the like.

As used herein, the terms “release” and “release rate” are used interchangeably to refer to the discharge or liberation, or rate thereof, of a substance, including without limitation a therapeutic agent, such as NO, from the dosage form or composition containing the substance. In one example, a therapeutic agent may be released in vitro. In another aspect, a therapeutic agent may be released in vivo.

As used herein, “immediate release” or “instant release” can be used interchangeably and refer to immediate or near immediate (i.e. uninhibited or unrestricted) release of an agent or substance, including a therapeutic agent, such as NO, from a composition or formulation.

As used herein, the term “controlled release” refers to non-immediate release of an agent or substance, including a therapeutic agent, such as NO, from a composition or formulation. Examples of specific types of controlled release include without limitation, extended or sustained release and delayed release. Any number of control mechanisms or components can be used to create a controlled release effect, including formulation ingredients or constituents, formulation properties or states, such as pH, an environment in which the formulation is placed, or a combination of formulation ingredients and an environment in which the formulation is placed. In one example, extended release can include release of a therapeutic agent at a level that is sufficient to provide a therapeutic effect or treatment for a non-immediate specified or intended duration of time.

As used herein, the term “modulate” refers to any change in biological state, i.e. increasing, decreasing, and the like.

As use herein with respect to physiologic levels of a given substance, the term “baseline” refers to a level or concentration of the substance in a subject prior to administration of an active agent. For example, the baseline level of viral RNA load in a subject would the subject's viral RNA load prior (e.g. just prior) to the commencement of NORS administration or therapy.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, the term “about” is used to provide flexibility to a numerical range endpoint by providing that a given value may be “a little above” or “a little below” the endpoint. Unless otherwise stated, use of the term “about” in accordance with a specific number or numerical range should also be understood to provide support for such numerical terms or range without the term “about”. For example, for the sake of convenience and brevity, a numerical range of “about 50 ml to about 80 ml” should also be understood to provide support for the range of “50 ml to 80 ml.” Furthermore, it is to be understood that in this specification support for actual numerical values is provided even when the term “about” is used therewith. For example, the recitation of “about” 30 should be construed as not only providing support for values a little above and a little below 30, but also for the actual numerical value of 30 as well.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually, and further including decimal or fraction values such as 1.8, 2.3, 3.7, and 4.2.

This same principle applies to ranges reciting only one numerical value as a minimum or a maximum. Furthermore, such an interpretation should apply regardless of the breadth of the range or the characteristics being described.

Reference throughout this specification to “an example” means that a particular feature, structure, or characteristic described in connection with the example is included in at least one embodiment. Thus, appearances of the phrases “in an example” in various places throughout this specification are not necessarily all referring to the same embodiment.

Reference in this specification may be made to devices, structures, systems, or methods that provide “improved” performance. It is to be understood that unless otherwise stated, such “improvement” is a measure of a benefit obtained based on a comparison to the prior art. Furthermore, it is to be understood that the degree of improved performance may vary between disclosed embodiments and that no equality or consistency in the amount, degree, or realization of improved performance is to be assumed as universally applicable.

As used herein, comparative terms such as “increased,” “decreased,” “better,” “worse,” “higher,” “lower,” “enhanced,” “improved,” “maximized,” “minimized,” and the like refer to a property of a device, component, composition, biologic response, biologic status, or activity that is measurably different from other devices, components, compositions, biologic responses, biologic status, or activities that are in a surrounding or adjacent area, that are similarly situated, that are in a single device or composition or in multiple comparable devices or compositions, that are in a group or class, that are in multiple groups or classes, or as compared to an original (e.g. untreated) or baseline state, or the known state of the art. For example, a composition that “decreases” viral load provides a viral load in a subject that is lower as compared to a viral load at a previous point in time, such as a baseline level (e.g. prior to treatment), or as compared to an earlier treatment with a different dose (e.g. lower dose).

### EXAMPLE EMBODIMENTS

An initial overview of invention embodiments is provided below and specific embodiments are then described in further detail. This initial summary is intended to aid readers in understanding the technological concepts more quickly, but is not intended to identify key or essential features thereof, nor is it intended to limit the scope of the claimed subject matter.

The clinical spectrum of infection from coronavirus disease 2019 (COVID-19) is wide, ranging from no symptoms, mild signs of upper respiratory tract infection, to severe pneumonia and death. COVID-19 is caused by the severe acute respiratory syndrome coronavirus 2 (SARS-CoV-2) virus. It is a single-stranded, positive-sense, RNA virus, which has a similar receptor-binding domain structure to that of SARS-CoV and MERS-CoV. The virus is transmitted via airborne droplets to the nasal mucosa. Rapid viral reproduction occurs within these cells and viral shedding into nasal secretions and sputum causes disease of the lower respiratory tract potentially causing fatal viral pneumonia.

Sputum may continue to contain the virus for almost 2 weeks after recovery and the disease can spread before onset of symptoms, during the symptomatic period and even after recovery. Infection of epithelial cells by the SARS-CoV-2 virus uses angiotensin converting enzyme 2 (ACE-2) as a receptor for cellular entry which is highly expressed in the nose. The binding affinity of the Spike protein to its cognate receptor ACE-2 is a major determinant of the SARS-CoV replication rate and disease severity.

Nitric oxide (NO) is a free radical gas molecule that plays a major role in innate immunity, mammalian host defense against infection, modulation of wound healing, vasodilation, neurotransmission and angiogenesis. NO has been reported to have antimicrobial activity against bacteria, yeast, fungi, and viruses both in vitro and in vivo in animal studies. NO also prevents the fusion between the Spike protein and ACE-2. Together, the antiviral and ACE-2 inhibitory effects of NO qualify it for a potential molecule to prevent and/or to minimize the severity of COVID-19 infections.

NORS provides antimicrobial chemical characteristics (in a solution) that can be equivalent to hours of exposure of 160 ppm NO gas. The NORS formulation has been developed and evaluated to enable a safe dose for host cells and can have a rapid microbicidal effect (within seconds) against bacterial, fungal and viral pathogens.

The present disclosure relates to the discovery that a liquid nitric oxide releasing solution (NORS) is effective in killing or otherwise deactivating SARS-CoV-2 and upper respiratory tract infections. Accordingly, it is thought that NORS can be used for the purpose of treating, preventing, or reducing its infection of individuals.

In one embodiment, a method of treating a subject for infection with a Severe Acute Respiratory Syndrome Coronavirus (SARSr-CoV) can include administering a therapeutically effective amount of a nitric oxide releasing solution (NORS) to the subject. In another embodiment, a method of minimizing subject-to-subject transmissibility for a pathogen can include administering a therapeutically effective amount of a nitric oxide releasing solution (NORS) to the subject.

In yet another embodiment, a method of treating a subject for infection of a pathogen in an upper respiratory tract can include administering a therapeutically effective amount of a nitric oxide releasing solution (NORS) to the subject as a spray having an average droplet volume which contains treatment within the upper respiratory tract. In one more embodiment, a nitric oxide releasing solution (NORS) can include at least one nitric oxide releasing compound and at least one acidifying agent. In one aspect, the NORS can release a therapeutically effective amount as a spray having an average droplet volume which contains treatment within the upper respiratory tract

### NORS Composition

In one embodiment, a nitric oxide releasing solution (NORS) can include at least one nitric oxide releasing compound and at least one acidifying agent. In one aspect, the NORS can be released as a spray having an average droplet volume which contains treatment within the upper respiratory tract or airway.

In some embodiments, the NORS can provide release (e.g. an extended release) of gaseous nitric oxide (gNO) when administered to a situs. By “extended release,” it is meant that an effective amount of NO gas is released from the formulation at a controlled rate such that therapeutically beneficial levels (but below toxic levels) of gNO are maintained over an extended period of time, ranging from, e.g., about 5 seconds to about 24 hours, thus, providing, for example, a 30 to 60 minute, or several hour, dosage form. In one embodiment, the NO gas is released over a period of at least 30 minutes. In a further embodiment, the NO gas is released over a period of at least 1 hour, or from about 1.5 hours to about 3 hours. In another embodiment, the NO gas is released over a period of at least 8 hours. In another embodiment, the NO gas is released over a period of at least 12 hours. In another embodiment, the NO gas is released over a period of at least 24 hours. An extended release NORS is beneficial in that the solution can be administered to a situs over a short period of time, while the release of NO from the solution continues following administration.

The solution of the present invention becomes active when the nitrites and acids mix in saline or water in which the pH of the solution is below about 4.0 and exhibits an increased or enhanced production level of nitric oxide gas over an extended period of time. In one embodiment, the pH of the active state of the nitric oxide releasing solution is between a pH of about 1.0 and a pH of about 4.0. In another embodiment, the pH of the active state of the nitric oxide releasing solution is between a pH of about 3.0 and a pH of about 4.0. In one embodiment, the pH is about 3.2. In another embodiment, the pH is about 3.4. In another embodiment, the pH is about 3.5. In another embodiment, the pH is about 3.6. In another embodiment, the pH is about 3.7. In one embodiment, the pH is about 4.0. In another embodiment, the pH is below about 4.0.

Because the nitric oxide releasing solution of the present invention is not active until the acid interacts with the nitrites in liquid, the nitrite solution can be pre-made, transported and set up for administration while in its dormant state (pH greater than 4.0), without producing any appreciable nitric oxide gas or without losing its ability to produce an effective amount of nitric oxide gas. Then, when a user is ready to deliver or administer the solution for treatment of a human subject, the solution can be activated immediately prior to administration to the human subject by the addition of an acid (pH driven below 4.0), thereby maximizing the amount of nitric oxide gas produced by the administered dosage of solution.

In one embodiment, the pH of the solution can be lowered via addition of at least one acidifying agent into the solution. Introduction of the acidifying agent drives the solution reaction towards the reactants, thus reducing the pH (creating more acid), which in turn creates more nitric oxide gas.

For example, by introducing sodium nitrite (or other salts of nitrites) to a saline solution it will very slowly produce nitric oxide gas, but in an undetectable amount (as measured by chemiluminescence analysis methodology (ppb sensitivity)). The rate of NO produced from the solution increases as the pH is decreased, particularly as it drops below pH 4.0. NO is produced based on the following equilibrium equations:

NO₂⁻+H⁺ [symbol] HNO₂ 1.

2HNO₂ [symbol] N₂O₃+H₂O [symbol] H₂O+NO+NO₂ 2a.

3HNO₂ [symbol] 2NO+NO₃⁻+H₂O+H⁺ 2b.

Therefore, an acidifying agent, for example an acid, may donate the H⁺ to the nitrite (NO₂⁻). The more H⁺ present, the faster the reaction will go towards HNO₂ and the more NO will be produced.

In one embodiment, the nitric oxide releasing solution includes the use of a water or saline-based solution and at least one nitric oxide releasing compound, such as nitrite or a salt thereof. In one embodiment, the solution is a saline-based solution. In one embodiment, the nitric oxide releasing compound is a nitrite, a salt thereof, and any combinations thereof. Non-limiting examples of nitrites include salts of nitrite such as sodium nitrite, potassium nitrite, barium nitrite, and calcium nitrite, mixed salts of nitrite such as nitrite orotate, and nitrite esters such as amyl nitrite. In one embodiment, the nitric oxide releasing compound is selected from the group consisting of sodium nitrite and potassium nitrite, and any combinations thereof. In another embodiment, the nitric oxide releasing compound is sodium nitrite. In one embodiment, the solution is comprised of sodium nitrite in a saline solution. In another embodiment, the solution is comprised of potassium nitrite in a saline solution.

In one embodiment, the concentration of nitrites in the solution is between 0.07% w/v and about 0.5% w/v. In one embodiment, the concentration of nitrites in the solution is no greater than about 0.5% w/v. In another embodiment, the concentration of nitrites in the solution is about 0.41% w/v. In another embodiment, the concentration of nitrites in the solution is between about 0.07-0.5% w/v. As used herein, the term “w/v” refers to the (weight of solute/volume of solution)×100%.

The acidifying agent can be any suitable acidifying agent. As described elsewhere here, the addition of at least one acidifying agent to the solution of the present invention contributes toward increased production of NO. Any acidifying agent which provides increased production of NO is contemplated by the present invention. In one embodiment, the acidifying agent is an acid, such as an inorganic or an organic acid. Non-limiting examples of acids include ascorbic acid, ascorbyl palmitate, salicylic acid, malic acid, lactic acid, citric acid, formic acid, benzoic acid, tartaric acid, hydrochloric acid, sulfuric acid, and phosphoric acid acetic acid and the like. In one embodiment, the acid is selected from the group consisting of ascorbic acid, citric acid, malic acid, hydrochloric acid, and sulfuric acid, and any combinations thereof. In one embodiment, the acid is citric acid.

As described above, the amount of acidifying agent present in the solution can affect the rate of the reaction to produce NO. In one embodiment, the amount of acidifying agent is no greater than about 0.5% w/v. In another embodiment, the amount of acidifying agent is about 0.5% w/v. In another embodiment, the amount of acidifying agent is about 0.2% w/v. In one embodiment, the amount of acidifying agent is about 0.07% w/v. In another embodiment, the amount of acidifying agent is between about 0.07-0.5% w/v.

In addition to affecting the rate of the NO-producing reaction, the acidifying agent can provide a low pH-level that reduces viral load (e.g., viral load of the SARS-CoV-2 virus). The increase in NO compared to baseline levels before treatment can also block viral access (e.g., SARS-CoV-2 viral access) to the host cell by enveloping epithelial cells with a physical barrier. The NO can further block viral access (e.g., SARS-CoV-2 access) to host cells through the ACE-2 receptors.

The NORS may release an effective or therapeutically effective concentration of NO. In one embodiment, the concentration of NO is between about 100 ppb and about 1000 ppm. In another embodiment, the concentration of NO is between about 120 ppb and about 400 ppb. In another embodiment, the therapeutically effective concentration of NO can be about 160 ppb. In another example, the concentration of NO is between about 100 ppb and 1000 ppb. In another example, the concentration of NO is between about 100 ppb and 500 ppb. In another example, the concentration of NO is between about 100 ppb and 300 ppb.

In another embodiment, the nitric oxide releasing solution can comprise a thickener, such as polyacrylic acids (e.g. Carbopols, for example), gelatin, pectin, tragacanth, methyl cellulose, hydroxylethylcellulose, hydroxypropylcellulose, HPMC, CMC, alginate, starch, polyvinyl alcohol, polyvinyl pyrrolidone, co-polymers of polyoxyethylene and polyoxypropylene, polyethylene glycol, the like, or combinations thereof. In one example, the thickener can comprise hydroxypropyl methylcellulose (HPMC).

In another embodiment, the nitric oxide releasing solution can comprise a preservative. Non-limiting examples of preservatives can include ascorbic acid, acetylcysteine, bisulfite, metabisulfite, monothioglycerol, phenol, meta-cresol, benzyl alcohol, methyl paraben, propyl paraben, butyl paraben, benzalkonium chloride, benzethonium chloride, butylated hydroxyl toluene, myristyl gamma-picolimium chloride, 2-phenoxyethanol, phenyl mercuric nitrate, chlorobutanol, thimerosal, tocopherols, the like, or combinations thereof.

In another embodiment, the nitric oxide releasing solution can comprise a tonicity agent. Non-limiting examples of tonicity agents include sodium chloride, potassium chloride, calcium chloride, magnesium chloride, mannitol, sorbitol, dextrose, glycerin, propylene glycol, ethanol, trehalose, phosphate-buffered saline (PBS), Dulbecco's PBS, Alsever's solution, Tris-buffered saline (TBS), water, balanced salt solutions (BSS), such as Hank's BSS, Earle's BSS, Grey's BSS, Puck's BSS, Simm's BSS, Tyrode's BSS, and BSS Plus, the like, or combinations thereof. In one embodiment, the tonicity agent can have sufficient NaCl to create an isotonic saline solution (e.g., 0.9% NaCl). In one aspect, the tonicity of the composition can be from about 250 to about 350 milliosmoles/liter (mOsm/L). In another aspect, the tonicity of the composition can be from about 277 to about 310 mOsm/L.

### Dosage Forms and Regimens

The NORS may be administered in a variety of forms. The NORS may be administered as a liquid, a spray, a vapor, micro-droplets, mist, gargle, lavage, aerosol, or any form which provides the release of nitric oxide from the solution. In one embodiment, the NORS is administered as a spray. The amount or dosing volume of administered nitric oxide releasing solution may be varied in order to maximize the duration of nitric oxide production and delivery. In one embodiment, the amount of nitric oxide releasing solution administered is between about 0.1 mL and 5000 mL. In another embodiment, the amount of nitric oxide releasing solution administered is between about 10 mL and 1000 mL. The nitric oxide releasing solution may be readministered one or more times, as used to effectively treat the situs.

When the NORS is administered as a spray, the amount of administered nitric oxide releasing solution can be from about 100 μl to about 1000 μl for each actuation of a spray device. In one aspect, the amount of administered nitric oxide releasing solution can be actuated from 1 to 6 times for each administration to provide a total amount per administration of from about 100 μl to about 5000 μl.

In one example, the amount of administered nitric oxide releasing solution can be from about 100 μl to about 200 μl for each actuation of a spray device. In this example, 100 μl to about 200 μl of NORS can be actuated 1 to 4 times for each administration to provide a total amount per administration of from about 100 μl to about 800 μl.

In another example, the amount of administered nitric oxide releasing solution can be from about 120 μl to about 140 μl for each actuation of a spray device. In this example, 120 μl to about 140 μl of NORS can be actuated 2 to 4 times for each administration to provide a total amount per administration of from about 240 μl to about 560 μl.

When the NORS is administered as a lavage, the amount of administered nitric oxide releasing solution can be from about 50 mL to about 500 mL. In one aspect, the amount of administered nitric oxide releasing solution can be from about 100 mL to about 400 mL. In another aspect, the amount of administered nitric oxide releasing solution can be from about 150 mL to about 350 mL.

When the NORS is administered as a gargle, the amount of administered nitric oxide releasing solution can be from about 5 mL to about 50 mL. In one aspect, the amount of administered nitric oxide releasing solution can be from about 10 mL to about 40 mL. In another aspect, the amount of administered nitric oxide releasing solution can be from about 10 mL to about 30 mL.

The NORS can be administered according to various dosage regimens. In one aspect, the NORS can be administered to the subject according to a dosage regimen of 1 to 6 times per day for a duration of from about a single day to about 2 weeks. In another aspect, the NORS can be administered to the subject according to a dosage regimen of 4 to 6 times per day for a duration of from about a single day to about 2 weeks. In yet another aspect, the NORS can be administered to the subject according to a dosage regimen of 5 to 6 times per day for a duration of from about a single day to about 2 weeks. In yet another aspect, the NORS can be administered to the subject according to a dosage regimen of 5 to 6 times per day for a duration of from about 1 day to about 9 days.

The NORS can be administered to a subject in varying dosage amounts. In one example, the NORS can be administered to the subject at a single dose of from about 300 μl of NORS to about 700 μl of NORS. In another example, the NORS can be administered to the subject at a single dose of from about 400 μl of NORS to about 600 μl of NORS.

The NORS can also be administered to a subject at a daily dose. In one example, the NORS can be administered to the subject at a daily dose of from about 1500 μl of NORS to about 4200 μl of NORS. In another example, the NORS can be administered to the subject at a daily dose of from about 2000 μl of NORS to about 3600 μl of NORS.

In certain embodiments, the nitric oxide releasing solution is prepared just prior to administration to the situs through the administration of an acidifying agent to a dormant solution. For example, as described elsewhere herein, administration of the acidifying agent to the dormant solution results in the lowering of the pH of the dormant solution, thereby activating the nitric oxide releasing solution to be administered to the treatment site.

The nitric oxide releasing solution provides for extended production of nitric oxide. In one embodiment, the nitric oxide releasing solution produces nitric oxide for a period of between 1 minute and 24 hours. In one embodiment, the nitric oxide releasing solution produces nitric oxide for a period of between 10 and 45 minutes. In one embodiment, the nitric oxide releasing solution produces nitric oxide for at least 15 minutes. In one embodiment, the nitric oxide releasing solution produces nitric oxide for at least 30 minutes. In another embodiment, the nitric oxide releasing solution produces nitric oxide for at least 1 hour. In another embodiment, the nitric oxide releasing solution produces nitric oxide for at least 4 hours. In another embodiment, the nitric oxide releasing solution produces nitric oxide for at least 8 hours. In another embodiment, the nitric oxide releasing solution produces nitric oxide for at least 12 hours. In another embodiment, the nitric oxide releasing solution produces nitric oxide for at least 24 hours. Thus, the administered nitric oxide releasing solution provides for continuous delivery of nitric oxide to the treatment site of the subject, or a location distal therefrom.

The NORS can be administered to an afflicted situs. In one aspect, the afflicted situs can be an upper respiratory tract or upper airway of the subject. In another aspect, the afflicted situs can be a mucosal membrane. When the afflicted situs is a mucosal membrane, the NORS can be administered to a subject's nasal passage or sinus and the NORS can be administered as a sprayed solution or as a lavage. In another example, when the mucosal membrane is a conjunctival mucosa of the subject, the NORS can be administered as an ophthalmic solution. In another example, when the afflicted situs is a mucosal membrane and the mucosal membrane is a subject's mouth or throat, the NORS can be administered to the afflicted situs as a gargle solution.

In some aspects, NORS can be administered to a site that is different from, including distal from, an afflicted situs, but which location still allows NO to reach the intended treatment site or afflicted situs to have a therapeutic effect. In one example, when the NORS is administered as a spray, a lavage, or a gargle, the NORS may not be administered to the upper airway of a subject, but can reach the upper airway to have a therapeutic effect.

In certain embodiments, nitric oxide releasing solution is directly administered into the upper respiratory tract of a subject. For example, in one embodiment, the nitric oxide releasing solution is sprayed into the upper respiratory tract of the subject. The solution may be administered into the upper respiratory tract of the subject once an hour, once a day, once a week, once every two weeks, once a month, once every two months, once a year, and any and all ranges therebetween as used to treat the subject. In one embodiment, the solution is sprayed once a week. In another embodiment, the solution is sprayed once a week for four consecutive weeks. The nitric oxide releasing solution provides for extended nitric oxide production, thereby providing continuous delivery of therapeutic nitric oxide to the upper respiratory infection of the subject.

In one embodiment, a nitric oxide releasing solution (NORS) can include at least one nitric oxide releasing compound and at least one acidifying agent. In one aspect, the NORS can release a therapeutically effective amount as a spray having an average droplet volume which contains treatment within an upper respiratory tract.

In one aspect, the spray can have a median droplet size (Dv50) of greater than one or more of 100 μm, 150 μm, 200 μm, 250 μm, 300 μm, 350 μm, 400 μm, 450 μm, 500 μm, 550 μm, or 600 μm when measured a 30 mm or 60 mm distance from actuation.

In another aspect, the spray can have a percentage of spray by volume at droplet sizes of less than 10 μm (%<10 μm) of less than one or more of: 0.5%, 0.4%, 0.3%, 0.2%, or 0.1% when measured a 30 mm or 60 mm distance from actuation. In another aspect, the spray can have a percentage of spray by volume at droplet sizes of less than 5 μm (%<5 μm) of less than one or more of: 0.5%, 0.4%, 0.3%, 0.2%, or 0.1% when measured a 30 mm or 60 mm distance from actuation.

In another aspect, the spray can have a 10^{th} percentile by volume of spray (Dv(10)) of greater than one or more of 100 μm, 150 μm, 200 μm, 250 μm, 300 μm, 350 μm, or 400 μm when measured a 30 mm or 60 mm distance from actuation. In another aspect, the spray can have a 90^{th} percentile by volume of spray (Dv(90)) of greater than one or more of 500 μm, 550 μm, 600 μm, 650 μm, 700 μm, 750 μm, 800 μm, 850 μm, 900 μm, 950 μm, or 1000 μm when measured a 30 mm or 60 mm distance from actuation. In another aspect, the spray can have a droplet size distribution of about 0.5 to about 2.0 (the droplet size distribution can be defined as (Dv(90)−Dv(10))/Dv(50)).

The duration of administering the nitric oxide releasing solution to the subject may be varied in order to maximize delivery. In one embodiment, the nitric oxide releasing solution is administered over a time period of less than 5 seconds. In another embodiment, the nitric oxide releasing solution is administered over a time period of about 5 seconds. In another embodiment, the nitric oxide releasing solution is administered over a time period of about 30 seconds. In another embodiment, the nitric oxide releasing solution is administered over a time period of about 1 minute to about 20 minutes.

### Methods

In one embodiment, a method of treating a subject for infection with a Severe Acute Respiratory Syndrome Coronavirus (SARSr-CoV) can include administering a therapeutically effective amount of a nitric oxide releasing solution (NORS) to the subject. In one aspect, the NORS can be administered to the subject when the subject is asymptomatic. In another aspect, the NORS can be administered to the subject when displaying mild symptoms of SARS-CoV-2 infection. In another aspect, the NORS can be administered to the subject when displaying moderate symptoms of SARS-CoV-2 infection. In another aspect, the NORS can be administered to the subject when displaying severe symptoms of SARS-CoV-2 infection. In another aspect, the NORS can be administered to the subject when displaying critical symptoms of SARS-CoV-2 infection.

A subject can be asymptomatic when the subject does not display mild, moderate, severe, or critical symptoms. A subject can display mild symptoms when the subject has non-pneumonia or mild pneumonia with no chest pain or shortness of breath, where pneumonia is defined as ‘inflammation of one or both lungs.’ Mild symptoms could include but were not limited to fever (>37.2 C), dry cough, tiredness, sore throat, malaise, headache, muscle pain, lack of taste or smell, and gastrointestinal symptoms.

A subject can display moderate symptoms when there is clinical or radiographic evidence of lower respiratory tract disease and when oxygen saturation is greater than or equal to 94%. A subject can display server symptoms when oxygen saturation is less than 94%, the respiratory rate is greater than or equal to 30 breaths-minute, and the lung has been infiltrated by greater than 50%. A subject can display critical symptoms when there is respiratory failure, shock, or multiorgan dysfunction or failure.

In some cases, administering a therapeutically effective amount of NORS to the subject can reduce the duration and severity of symptoms compared to the duration of the symptoms when NORS is not administered to a subject. For example, administering the therapeutically effective amount of NORS can provide these benefits by reducing the viral load of the subject.

In another aspect, the NORS can be administered to the subject within a selected number of days of an identified first-person exposure. In another aspect, the NORS can be administered to the subject without an identified first-person exposure.

In another aspect, the NORS can be administered to the subject when displaying acute symptoms of SARS-CoV-2 infection. In another aspect, the NORS can be administered to the subject when displaying chronic symptoms of SARS-CoV-2 infection.

Treatment of a respiratory disease by way of the present invention can comprise the delivery of a nitric oxide releasing solution into the upper respiratory tract of the subject to be treated. For example, in certain embodiments, the nitric oxide releasing solution may be injected, sprayed, inhaled, or instilled into the respiratory tract of the subject. In one embodiment, the nitric oxide releasing solution may be administered to the respiratory tract of the subject using a nitric oxide nasal spray (NONS) via the nasal cavity or oral cavity of the subject. One specific example of such a NONS is shown in Applicant's copending U.S. Provisional Patent Application Ser. No. 63/079,277, filed on Sep. 16, 2020, which incorporated herein by reference in its entirety. In one embodiment, the nitric oxide releasing solution is sprayed into the upper respiratory tract of the subject. In one embodiment, the solution is administered to the subject intranasally. In one embodiment, the solution is administered to the sinuses. The nitric oxide releasing solution provides for extended nitric oxide production, thereby providing continuous delivery of therapeutic nitric oxide to the upper respiratory tract of the subject.

In one example, the method can comprise the treatment, prevention, or reduction of incidence of a virally induced condition. The virally induced condition can be caused by or associated with one or more of: adenovirus, influenza, enteroviruses, human metapneumoviruses, astrovirus, rhinovirus, respiratory syncytial virus, parainfluenza, severe acute respiratory syndrome (SARS), coronavirus, H1N1, H2N2, H3N2, H1N1pdm09, or combinations thereof. In one example, the virally induced condition can be caused by or associated with one or more of human coronavirus 229E, human coronavirus OC43, human coronavirus HKU1, human coronavirus NL63, MERS-coronavirus, human respirovirus 1, human rubulavirus 2, human respirovirus 3, human rubulavirus 4, human enterovirus, human respiratory virus, rhinovirus A, rhinovirus B, rhinovirus C, or combinations thereof. In one aspect, influenza can be any of Influenza A, Influenza B, Influenza C, or Influenza D.

In another example, the method can comprise the treatment, prevention, or reduction of incidence of a bacterial condition. The bacterial condition can be one or more of: Bacillus, Bartonella, Bordetella, Borrelia, Brucella, Campylobacter, Chlamydia, Chlamydophila, Clostridium, Corynebacterium, Enterococcus, Escherichia, Francisella, Haemophilus, Helicobacter, Legionella, Leptospira, Listeria, Mycobacterium, Mycoplasma, Neisseria, Pseudomonas, Rickettsia, Salmonella, Shigella, Staphylococcus, Streptococcus, Treponema, Ureaplasma, Vibrio, Yersinia, the like, and combinations thereof.

In another example, the method can comprise the treatment, prevention, or reduction of incidence of a fungal condition. The fungal condition can be caused by a fungus selected from a genus including: Aspergillus, Histoplasma, Pneumocystis, Stachybotrys, the like, and combinations thereof.

In one embodiment, the method comprises the treatment, prevention, or reduction of incidence of a Severe Acute Respiratory Syndrome Coronavirus (SARSr-CoV). In one embodiment, administration of NORS can treat, prevent, or reduce the incidence of SARS-CoV-2 or a variant thereof. In one aspect, the SARS-CoV-2 virus can be one or more of: Lineage A (also termed as the sequence WIV04/2019), Cluster 5 (also termed as the ΔFVI-spike by the Danish State Serum Institute), Lineage B.1.1.7 (also termed as the UK variant, the variant under investigation (VUI) 202012/01, or 201/501Y.V1), B.1.1.7 with E484K (also termed as the variant of concern 202102/02 (VOC 202102/02), Lineage B.1.1.207, Lineage B.1.1.317, Lineage B.1.1.318 (VUI-202102/04), Lineage B.1.351 (also termed as the 501.V2 variant, 20H/501Y.V2, or variant of concern (VOC-202012/02), Lineage B.1.429/CAL.20C, Lineage B.1.525 (also termed as VUI-202102/03 and formerly termed as UK 1188), Lineage P.1 (VOC 202101/02), Lineage B.1.427 (also termed as 20C/S:452R), Lineage B.1.526, Lineage P.2, or a combination thereof.

In another embodiment, administration of NORS can provide a treatment, prevention, or reduction of incidence of SARS-CoV-2 or a variant thereof that can be substantially equally effective for treating one or more of: Lineage A, Cluster 5, Lineage B.1.1.7, B.1.1.7 with E484K, Lineage B.1.1.207, Lineage B.1.1.317, Lineage B.1.1.318, Lineage B.1.351, Lineage B.1.429/CAL.20C, Lineage B.1.525, Lineage P.1, Lineage B.1.427, Lineage B.1.526, Lineage P.2, or a combination thereof. In one example, administration of NORS can be substantially equally effective in treating SARS-CoV-2 or a variant thereof when the viral load of SARS-CoV-2 or the variant is reduced by more than 80%, 90%, or 95%, or 99% in a subject within 24 hours, or 48 hours, or 72 hours, or 96 hours, or 120 hours, or 148 hours of treatment compared to a baseline viral load of SARS-CoV-2 or the variant before the commencement of treatment for SARS-CoV-2 or for the variant.

The NORS can reduce the viral load compared to a baseline level. In one aspect, the therapeutically effective amount of the NORS can reduce a viral RNA load compared to a baseline level by greater than one or more of: 80%, 90%, 95%, or 99% after a treatment duration of one or more of: 1 day, 2 days, 3 days, 4 days, 5 days, or 6 days.

The NORS can reduce the viral load through various mechanisms. In one mechanism, the NORS can provide a low pH (e.g., a pH of about 3.5) that can reduce the viral load (e.g., SARS-CoV-2 viral load). In another mechanism, the NORS can provide a physical barrier over epithelial cells of the subject that can prevent access of the virus to the host cells of the subject. In one example, when the virus is SARS-CoV-2, the NORS can provide a physical barrier that prevents the SARS-CoV-2 virus from accessing host cells of the subject. In another mechanism, the NORS can provide an angiotensin-converting enzyme 2 (ACE-2) receptor blocking agent.

NORS can also be used in a method of minimizing subject-to-subject transmissibility for a pathogen. In one example, a therapeutically effective amount of a nitric oxide releasing solution (NORS) can be administered to a subject. The NORS can be administered to the subject before or after exposure of the subject to the pathogen. The NORs can also be administered to the subject before or after exposure of the subject to a person displaying symptoms of the pathogen.

The pathogen can include any pathogen disclosed herein. For example, the pathogen can be a Severe Acute Respiratory Syndrome Coronavirus (SARSr-CoV) or a variant thereof. The SARSr-CoV can be a SARS-CoV-2 virus including one or more of: Lineage A, Cluster 5, Lineage B.1.1.7, B.1.1.7 with E484K, Lineage B.1.1.207, Lineage B.1.1.317, Lineage B.1.1.318, Lineage B.1.351, Lineage B.1.429/CAL.20C, Lineage B.1.525, Lineage P.1, Lineage B.1.427, Lineage B.1.526, Lineage P.2, the like, or a combination thereof. In one example, the therapeutically effective amount of the NORS can be substantially equally effective for minimizing subject-to-subject transmissibility for a a Severe Acute Respiratory Syndrome Coronavirus (SARSr-CoV) or a variant thereof including one or more of: Lineage A, Cluster 5, Lineage B.1.1.7, B.1.1.7 with E484K, Lineage B.1.1.207, Lineage B.1.1.317, Lineage B.1.1.318, Lineage B.1.351, Lineage B.1.429/CAL.20C, Lineage B.1.525, Lineage P.1, Lineage B.1.427, Lineage B.1.526, Lineage P.2, the like, or a combination thereof.

The NORS can minimize subject-to-subject transmissibility by reducing the viral load of the pathogen. In one example, the therapeutically effective amount of the NORS can reduce a viral RNA load compared to a baseline level by greater than one or more of: 80%, 90%, 95%, or 99% after a treatment duration of one or more of: 1 day, 2 days, 3 days, 4 days, 5 days, or 6 days. The NORS can reduce the viral load through various mechanisms discussed herein including a low pH, a physical barrier over epithelial cells of the subject that prevents viral access to host cells of the subject, or an ACE-2 receptor blocking agent.

In some instances, method of treatment can involve NORS that can be constrained to an upper airway of a subject. In one embodiment, a method of treating a subject for infection of a pathogen in an upper respiratory tract can comprise administering a therapeutically effective amount of a nitric oxide releasing solution (NORS) to the subject as a spray (e.g. from a NONS) having an average droplet volume which contains treatment within the upper respiratory tract. Droplet size can be controlled by employing specific structures with the NONS. In this example, the NORS can remain in the nasal cavity and upper airways without substantial dispersion into the lungs of the subject. In such a case, there may be no detectable systemic increase in NO metabolites (e.g., methemoglobin) when NORS has been administered to the subject.

In order to reduce dispersion of the NORS and NO beyond the upper airway of the subject, the method can comprise providing a median droplet size (Dv50) of greater than one or more of 100 μm, 150 μm, 200 μm, 250 μm, 300 μm, 350 μm, 400 μm, 450 μm, 500 μm, 550 μm, or 600 μm when measured a 30 mm or 60 mm distance from actuation.

In another example, the distribution of droplet size can be minimal below a certain value. For example, the method can comprise providing a percentage of spray by volume at droplet sizes of less than 10 μm (%<10 μm) of less than one or more of: 0.5%, 0.4%, 0.3%, 0.2%, or 0.1% when measured a 30 mm or 60 mm distance from actuation. In another example, the method can comprise providing a percentage of spray by volume at droplet sizes of less than 5 μm (%<5 μm) of less than one or more of: 0.5%, 0.4%, 0.3%, 0.2%, or 0.1% when measured a 30 mm or 60 mm distance from actuation.

The distribution of droplet size can be bounded at the 10^{th} percentile and the 90^{th} percentile. In one example, the method can comprise providing a 10^{th} percentile by volume of spray (Dv(10)) of greater than one or more of 100 μm, 150 μm, 200 μm, 250 μm, 300 μm, 350 μm, or 400 μm when measured a 30 mm or 60 mm distance from actuation. In another example, the method can comprise providing a 90^{th} percentile by volume of spray (Dv(90)) of greater than one or more of 500 μm, 550 μm, 600 μm, 650 μm, 700 μm, 750 μm, 800 μm, 850 μm, 900 μm, 950 μm, or 1000 μm when measured a 30 mm or 60 mm distance from actuation.

The span of the distribution of the droplet size can be calculated from the 90^{th} percentile droplet size, the 10^{th} percentile droplet size, and the median droplet size. The method can comprise providing a droplet size distribution of about 0.5 to about 2.0, wherein the droplet size distribution is: (Dv(90)−Dv(10))/Dv(50).

In one embodiment, a composition for use in treating a subject for infection with a Severe Acute Respiratory Syndrome Coronavirus (SARSr-CoV) can comprise administering a therapeutically effective amount of nitric oxide releasing solution (NORS) to the subject. In another embodiment, a composition for use in minimizing subject-to-subject transmissibility for a pathogen can comprise administering a therapeutically effective amount of nitric oxide releasing solution (NORS) to the subject. In yet another embodiment, a composition for use in treating a subject for infection of a pathogen in an upper respiratory tract can comprise administering a therapeutically effective amount of nitric oxide releasing solution (NORS) to the subject as a spray having an average droplet volume which contains treatment within the upper respiratory tract.

In another embodiment, the use of a pharmaceutical composition for the manufacture of a medicament for treating a subject for infection with a Severe Acute Respiratory Syndrome Coronavirus (SARSr-CoV) can comprise administering a therapeutically effective amount of nitric oxide releasing solution (NORS) to the subject. In another embodiment, the use of a pharmaceutical composition for the manufacture of a medicament for minimizing subject-to-subject transmissibility for a pathogen can comprise administering a therapeutically effective amount of nitric oxide releasing solution (NORS) to the subject. In another embodiment, the use of a pharmaceutical composition for the manufacture of a medicament for treating a subject for infection of a pathogen in an upper respiratory tract can comprise administering a therapeutically effective amount of nitric oxide releasing solution (NORS) to the subject as a spray having an average droplet volume which contains treatment within the upper respiratory tract.

### EXAMPLE EMBODIMENTS

In one example, a method of treating a SARS-CoV-2 virus can comprise administering an effective amount of a nitric oxide releasing solution (NORS) to a location where the SARS-CoV-2 virus resides.

In one example, the SARS-CoV-2 virus can reside on an exposed surface of an object.

In one example, the SARS-CoV-2 virus can reside on an exterior surface of a subject.

In one example, the SARS-CoV-2 virus can reside within a tissue of a subject.

In one example, the tissue can be a mucosal tissue.

In one example, the SARS-CoV-2 virus can be one or more of: Lineage A, Cluster 5, Lineage B.1.1.7, B.1.1.7 with E484K, Lineage B.1.1.207, Lineage B.1.1.317, Lineage B.1.1.318, Lineage B.1.351, Lineage B.1.429/CAL.20C, Lineage B.1.525, Lineage P.1, Lineage B.1.427, Lineage B.1.526, Lineage P.2, or a combination thereof.

In one example, the effective amount of the NORS can be substantially equally effective for treating one or more of: Lineage A, Cluster 5, Lineage B.1.1.7, B.1.1.7 with E484K, Lineage B.1.1.207, Lineage B.1.1.317, Lineage B.1.1.318, Lineage B.1.351, Lineage B.1.429/CAL.20C, Lineage B.1.525, Lineage P.1, Lineage B.1.427, Lineage B.1.526, Lineage P.2, or a combination thereof.

In one example, a method of treating a subject for infection with a Severe Acute Respiratory Syndrome Coronavirus (SARSr-CoV) can comprise administering a therapeutically effective amount of a nitric oxide releasing solution (NORS) to the subject.

In one example, the method can comprise administering the NORS to the subject when the subject is asymptomatic.

In one example, the method can comprise administering the NORS to the subject within a selected number of days of an identified first-person exposure.

In one example, the method can comprise administering the NORS to the subject without an identified first-person exposure.

In one example, the method can comprise administering the NORS to the subject when displaying mild symptoms of SARS-CoV-2 infection.

In one example, the method can comprise administering the NORS to the subject when displaying moderate symptoms of SARS-CoV-2 infection.

In one example, the method can comprise administering the NORS to the subject when displaying severe symptoms of SARS-CoV-2 infection.

In one example, the method can comprise administering the NORS to the subject when displaying critical symptoms of SARS-CoV-2 infection.

In one example, the method can comprise administering the NORS to the subject according to a dosage regimen of 1 to 6 times per day for a duration of from about a single day to about 2 weeks.

In one example, the method can comprise administering the NORS to the subject at a single dose of from about 300 ul of NORS to about 700 ul of NORS.

In one example, the method can comprise administering the NORS to the subject at a single dose of from about 400 ul of NORS to about 600 ul of NORS.

In one example, the method can comprise administering the NORS to the subject at a daily dose of from about 1500 ul of NORS to about 4200 ul of NORS.

In one example, the method can comprise administering the NORS to the subject at a daily dose of from about 2000 ul of NORS to about 3600 ul of NORS.

In one example, the therapeutically effective amount of the NORS can reduce a viral RNA load compared to a baseline level by greater than one or more of: 80%, 90%, 95%, or 99% after a treatment duration of one or more of: 1 day, 2 days, 3 days, 4 days, 5 days, or 6 days.

In one example, the therapeutically effective amount of the NORS can: provide a low pH that reduces SARS-CoV-2 viral load, or provide a physical barrier over epithelial cells of the subject that prevents SARS-CoV-2 access to host cells of the subject, or provide an angiotensin-converting enzyme 2 (ACE-2) receptor blocking agent.

In one example, the NORS can be administered to an afflicted situs.

In one example, the afflicted situs can be an upper respiratory tract of the subject.

In one example, the afflicted situs can be a mucosal membrane.

In one example, the mucosal membrane can be a subject's nasal passage or sinus and the NORS is administered as a sprayed solution or as a lavage.

In one example, the mucosal membrane can be a subject's mouth or throat and the NORS is administered as a gargle solution.

In one example, the virus can be a SARS-CoV-2 virus or a variant thereof.

In one example, the SARS-Co-V-2 virus or the variant thereof can be one or more of: Lineage A, Cluster 5, Lineage B.1.1.7, B.1.1.7 with E484K, Lineage B.1.1.207, Lineage B.1.1.317, Lineage B.1.1.318, Lineage B.1.351, Lineage B.1.429/CAL.20C, Lineage B.1.525, Lineage P.1, Lineage B.1.427, Lineage B.1.526, Lineage P.2, or a combination thereof.

In one example, the therapeutically effective amount of the NORS can be substantially equally effective for treating one or more of: Lineage A, Cluster 5, Lineage B.1.1.7, B.1.1.7 with E484K, Lineage B.1.1.207, Lineage B.1.1.317, Lineage B.1.1.318, Lineage B.1.351, Lineage B.1.429/CAL.20C, Lineage B.1.525, Lineage P.1, Lineage B.1.427, Lineage B.1.526, Lineage P.2, or a combination thereof.

In one example, a method of minimizing subject-to-subject transmissibility for a pathogen can comprise administering a therapeutically effective amount of a nitric oxide releasing solution (NORS) to the subject.

In one example, the method can comprise administering the NORS to the subject before or after exposure of the subject to the pathogen.

In one example, the method can comprise administering the NORS to the subject before or after exposure of the subject to a person displaying symptoms of the pathogen.

In one example, the pathogen can be a Severe Acute Respiratory Syndrome Coronavirus (SARSr-CoV) or a variant thereof.

In one example, the Severe Acute Respiratory Syndrome Coronavirus (SARSr-CoV) can be a SARS-CoV-2 virus including one or more of: Lineage A, Cluster 5, Lineage B.1.1.7, B.1.1.7 with E484K, Lineage B.1.1.207, Lineage B.1.1.317, Lineage B.1.1.318, Lineage B.1.351, Lineage B.1.429/CAL.20C, Lineage B.1.525, Lineage P.1, Lineage B.1.427, Lineage B.1.526, Lineage P.2, or a combination thereof.

In one example, the therapeutically effective amount of the NORS can be substantially equally effective for minimizing subject-to-subject transmissibility for a a Severe Acute Respiratory Syndrome Coronavirus (SARSr-CoV) or a variant thereof including one or more of: Lineage A, Cluster 5, Lineage B.1.1.7, B.1.1.7 with E484K, Lineage B.1.1.207, Lineage B.1.1.317, Lineage B.1.1.318, Lineage B.1.351, Lineage B.1.429/CAL.20C, Lineage B.1.525, Lineage P.1, Lineage B.1.427, Lineage B.1.526, Lineage P.2, or a combination thereof.

In one example, the therapeutically effective amount of the NORS can reduce a viral RNA load compared to a baseline level by greater than one or more of: 80%, 90%, 95%, or 99% after a treatment duration of one or more of: 1 day, 2 days, 3 days, 4 days, 5 days, or 6 days.

In one example, the therapeutically effective amount of the NORS can: provide a low pH that reduces SARS-CoV-2 viral load, or provide a physical barrier over epithelial cells of the subject that prevents SARS-CoV-2 access to host cells of the subject, or provide an angiotensin-converting enzyme 2 (ACE-2) receptor blocking agent.

In one example, wherein the NORS can be administered to a mucosal membrane.

In one example, the mucosal membrane can be a subject's nasal passage or sinus and the NORS is administered as a sprayed solution or as a lavage.

In one example, the mucosal membrane can be a subject's mouth or throat and the NORS is administered as a gargle solution.

In one example, a method of treating a subject for infection of a pathogen in an upper respiratory tract can comprise administering a therapeutically effective amount of a nitric oxide releasing solution (NORS) to the subject as a spray having an average droplet volume which contains treatment within the upper respiratory tract.

In one example, the method can comprise providing a median droplet size (Dv50) of greater than one or more of 100 μm, 150 μm, 200 μm, 250 μm, 300 μm, 350 μm, 400 μm, 450 μm, 500 μm, 550 μm, or 600 μm when measured a 30 mm or 60 mm distance from actuation.

In one example, the method can comprise providing a percentage of spray by volume at droplet sizes of less than 10 μm (%<10 μm) of less than one or more of: 0.5%, 0.4%, 0.3%, 0.2%, or 0.1% when measured a 30 mm or 60 mm distance from actuation.

In one example, the method can comprise providing a percentage of spray by volume at droplet sizes of less than 5 μm (%<5 μm) of less than one or more of: 0.5%, 0.4%, 0.3%, 0.2%, or 0.1% when measured a 30 mm or 60 mm distance from actuation.

In one example, the method can comprise providing a 10th percentile by volume of spray (Dv(10)) of greater than one or more of 100 μm, 150 μm, 200 μm, 250 μm, 300 μm, 350 μm, or 400 μm when measured a 30 mm or 60 mm distance from actuation.

In one example, the method can comprise providing a 90th percentile by volume of spray (Dv(90)) of greater than one or more of 500 μm, 550 μm, 600 μm, 650 μm, 700 μm, 750 μm, 800 μm, 850 μm, 900 μm, 950 μm, or 1000 μm when measured a 30 mm or 60 mm distance from actuation.

In one example, the method can comprise providing a droplet size distribution of about 0.5 to about 2.0, wherein the droplet size distribution is: (Dv(90)−Dv(10))/Dv(50).

In one example, a nitric oxide releasing solution (NORS) can comprise at least one nitric oxide releasing compound and at least one acidifying agent, wherein the NORS releases a therapeutically effective amount as a spray having an average droplet volume which contains treatment within an upper respiratory tract.

In one example, the NORS can provide a median droplet size (Dv50) of greater than one or more of 100 μm, 150 μm, 200 μm, 250 μm, 300 μm, 350 μm, 400 μm, 450 μm, 500 μm, 550 μm, or 600 μm when measured a 30 mm or 60 mm distance from actuation.

In one example, the NORS can provide a percentage of spray by volume at droplet sizes of less than 10 μm (%<10 μm) of less than one or more of: 0.5%, 0.4%, 0.3%, 0.2%, or 0.1% when measured a 30 mm or 60 mm distance from actuation.

In one example, the NORS can provide a percentage of spray by volume at droplet sizes of less than 5 μm (%<5 μm) of less than one or more of: 0.5%, 0.4%, 0.3%, 0.2%, or 0.1% when measured a 30 mm or 60 mm distance from actuation.

In one example, the NORS can provide a 10th percentile by volume of spray (Dv(10)) of greater than one or more of 100 μm, 150 μm, 200 μm, 250 μm, 300 μm, 350 μm, or 400 μm when measured a 30 mm or 60 mm distance from actuation.

In one example, the NORS can provide a 90th percentile by volume of spray (Dv(90)) of greater than one or more of 500 μm, 550 μm, 600 μm, 650 μm, 700 μm, 750 μm, 800 μm, 850 μm, 900 μm, 950 μm, or 1000 μm when measured a 30 mm or 60 mm distance from actuation.

In one example, the NORS can provide a droplet size distribution of about 0.5 to about 2.0, wherein the droplet size distribution is: (Dv(90)−Dv(10))/Dv(50).

In one example, the at least one nitric oxide releasing compound can be selected from the group consisting of a nitrite, a salt thereof, and any combinations thereof.

In one example, the at least one acidifying agent can be an acid.

In one example, a composition for use in treating a subject for infection with a Severe Acute Respiratory Syndrome Coronavirus (SARSr-CoV) can comprise administering a therapeutically effective amount of nitric oxide releasing solution (NORS) to the subject.

In one example, a composition for use in minimizing subject-to-subject transmissibility for a pathogen can comprise administering a therapeutically effective amount of nitric oxide releasing solution (NORS) to the subject.

In one example, a composition for use in treating a subject for infection of a pathogen in an upper respiratory tract can comprise administering a therapeutically effective amount of nitric oxide releasing solution (NORS) to the subject as a spray having an average droplet volume which contains treatment within the upper respiratory tract.

In one example, use of a pharmaceutical composition for the manufacture of a medicament for treating a subject for infection with a Severe Acute Respiratory Syndrome Coronavirus (SARSr-CoV) can comprise administering a therapeutically effective amount of nitric oxide releasing solution (NORS) to the subject.

In one example, use of a pharmaceutical composition for the manufacture of a medicament for minimizing subject-to-subject transmissibility for a pathogen can comprise: administering a therapeutically effective amount of nitric oxide releasing solution (NORS) to the subject.

In one example, use of a pharmaceutical composition for the manufacture of a medicament for treating a subject for infection of a pathogen in an upper respiratory tract can comprise administering a therapeutically effective amount of nitric oxide releasing solution (NORS) to the subject as a spray having an average droplet volume which contains treatment within the upper respiratory tract.

### EXPERIMENTAL EXAMPLES

The following examples are provided to promote a more clear understanding of certain embodiments of the present invention, and are in no way meant as a limitation thereon.

### EXAMPLE 1—Virucidal Assay

Testing was performed to determine if liquid samples inactivate virus when exposed for a contact time of 2 minutes or 8 minutes. Virus solution was mixed with samples for the liquid contact period, then surviving infectious virus was quantified by standard endpoint dilution and compared with untreated controls.

### Procedures:

### Virus, Media and Cells

SARS-CoV-2 stocks were prepared prior to testing by growing in Vero 76 cells in MEM supplemented with 2% FBS and 50 ug/mL gentamicin (test media).

### Virucidal Assay

Product G (nitrite) and Product H (acidifying agent) were mixed together at a 1:1 ratio prior to testing (G+H). The Product G+H produced a NO concentration of about 0.7 ppm to about 4 ppm with the area under the curve (AUC) after two minutes of from 0.7 ppm to about 2.0 ppm*min. Product J (nitrite) and Product K (acidifying agent) were likewise mixed (J+K). The Product J+K produced a NO concentration of from about 1.4 ppm to about 8 ppm with an area under the curve after two minutes of from about 1.4 ppm to about 4.0 ppm*min. Immediately after combining test samples, virus solution was added to the mixed samples at a 1:9 ratio (10 μL of virus in 90 μL of pre-mixed test samples). The mixtures were incubated together for 2 minutes or 8 minutes at room temperature. An untreated virus control of water and a positive control of EtOH (63%) were tested in parallel. For toxicity controls, media (no virus) was added to each sample. The test was performed in triplicate tubes. Following the contact period, samples were diluted 1/10 in test media and then stored at −80° C. until time of virus quantification.

### Virus Quantification

Surviving virus from each sample was quantified by standard CCID50 end-point dilution assay. Samples were thawed and serially diluted 1/10 in test medium. Then 100 NL of each dilution were plated into quadruplicate wells of 96-well plates containing 80-90% confluent Vero 76 cells. Plates were incubated at 37±2° C. with 5% CO2 for 6 days. Each well was then scored for presence or absence of virus. The CCID50 values were calculated using the Reed-Muench (1948) equation. Three independent replicates of each sample were tested, and the average and standard deviation were calculated. Results were compared with untreated controls by one-way ANOVA with Dunnett's multiple comparison tests using GraphPad Prism (version 8) software.

### Controls

Virus controls were tested in water and the reduction of virus in test wells compared to virus controls was calculated as the log reduction value (LRV). Toxicity controls were tested with media not containing virus to see if the samples were toxic to cells. Neutralization controls were tested to ensure that virus inactivation did not continue after the specified contact time, and that residual sample in the titer assay plates did not inhibit growth and detection of surviving virus. This was done by adding toxicity samples to titer test plates then spiking each well with a low amount of virus that would produce an observable amount of CPE during the incubation period.

### Results

Table 1 shows results for SARS-CoV-2 titers and LRV after contact with G+H and J+K liquid samples. After a 2-minute contact time, G+H reduced virus by from 3.9 to 1.8 log CCID50 per 0.1 mL (>99%). After 10 minutes, G+H reduced virus below the limit of detection, <0.7 CCID50 per 0.1 mL (>99.9%). After the 2-minute and 10-minute contact time, J+K reduced virus below the limit of detection of 0.7 CCID50 per 0.1 mL (>99.9%). Residual test sample did not inhibit virus growth and detection in the endpoint titer assays. Ethanol was cytotoxic in culture after the 1/10 dilution in media and therefore the lower limit of detection of virus was 1.7 CCID50 per well. Virus controls and positive controls performed as expected.

TABLE 1
Virucidal activity of G + H and J + K against SARS-
CoV-2 after contact period at 22□2□C.

| | | | Virus | |
|---|---|---|---|---|
| | Contact | | CCID₅₀ per | |
| Sample | Time | | well □ SD^{a} | LRV^{b} |
| G + H | 2 | minutes | 1.8 □ 0.46**** | 2.1 |
| J + K | 2 | minutes | <0.7**** | >3.2 |
| EtOH | 2 | minutes | <1.7**** | >2.2 |
| (63%)^{c} | | | | |
| Virus | 2 | minutes | 3.9 □ 0.19 | — |
| Control | | | | |
| G + H | 10 | minutes | <0.7**** | >3.2 |
| J + K | 10 | minutes | <0.7**** | >3.2 |
| EtOH | 10 | minutes | <1.7**** | >2.2 |
| (63%)^{c} | | | | |
| Virus | 10 | minutes | 3.9 □ 0.19 | — |
| Control | | | | |

^{a}Average of 3 replicates □ standard deviation
^{b}LRV (log reduction value) is the log₁₀ reduction of virus compared to the virus control
^{c}Ethanol was toxic to cells at 1/10 dilution and therefore the limit of detection of virus was <1.7 log
CCID50 per 0.1 mL.
****P < 0.0001 by one-way ANOVA with Dunnett's multiple comparison test, compared with untreated control (water).

### EXAMPLE 2—NONS Reduces SARS-CoV-2 Viral Titers in Hamsters

In a recent hamster study (N=12) conducted at Colorado State University, animals were inoculated with SARS-CoV-2. Half of the animals received NONS and half received no intervention. The intervention group received two treatments of nasal spray per day post-infection for three days. The NONS intervention was administered to the hamsters at the same dose as the proposed human dose (0.11 ppm) as a nasal spray. There was a reduction of viral titer on day 1 of approximately 2 logs compared to the control. Additionally, but not reflected in the graph, three of the six treated hamsters had titers at non-detectable levels and maintained low counts over the treatment period. The research team at Colorado State University confirmed that the controls were robust and additionally commented that to date, no other intervention evaluated in their laboratory against SARS-CoV-2 has produced such a marked decline on Day 1. More studies are being conducted with extended use of the nasal spray (up to 5 times daily).

### EXAMPLE 3-A—Overview of Clinical Study

### Summary

In a randomized, double-blind, placebo-controlled Phase II-b clinical trial that evaluated 80 confirmed cases of COVID-19, a therapeutically effective amount of NORS succeeded in significantly reducing the level of SARS-CoV-2, including in patients with high viral loads. The majority of these patients had been infected with the UK variant (e.g., Lineage B.1.1.7 or Variant Under Investigation 202012/01), which is considered a variant of concern. There were no adverse health events recorded in the UK trial, or in over 7,000 self-administered treatments given in earlier Canadian clinical trials.

The study was conducted to determine the clinical efficacy of NORS for the treatment of mild COVID-19 infection. The outcome measure was the difference in SARS-CoV-2 RNA load from baseline through Day 6 between NORS and control arms.

The study concluded that NORS accelerated clearance of SARS-CoV-2 by a factor of 16-fold versus the placebo, and it presents supporting evidence for the emergency use of NORS for prevention or treatment of patients with recent or established SARS-CoV-2 RNA infection. The study also concluded that patients with a self-administered nasal spray of NORS were found to have reduced SARS-CoV-2 log viral load by more than 95% in infected participants within 24 hours of treatment, and by more than 99% in 72 hours.

### Methods

### Study Design

This was a double-blinded, placebo-controlled phase IIb clinical trial to evaluate the use of NONS for the treatment of COVID-19 in individuals with mild COVID-19 infection. The trial was carried out at Ashford and St. Peter's Hospitals NHS Foundation Trust (ASPHFT). Ethical approval was obtained from the NHS Health Research Authority. The trial was carried out in accordance with the Good Clinical Practice (GCP) and principles of the Declaration of Helsinki and the ethical guidelines of the Council for International Organizations of Medical Sciences.

### Participants

Eligible participants were men and women aged 18 to 70 years with mild COVID-19 infection within 5 days of symptom onset and confirmed by a laboratory SARS-CoV-2 reverse transcription polymerase chain reaction (RT-PCR) nasal and throat swab within the 48 hours prior to randomization. It is intended that patients over 70 years of age will be tested in a further sub-analysis. ‘Mild’ COVID-19 infection was defined by non-pneumonia or mild pneumonia with no chest pain or shortness of breath, where pneumonia is defined as ‘inflammation of one or both lungs.’ Mild symptoms could include but were not limited to: fever (>37.2 C), dry cough, tiredness, sore throat, malaise, headache, muscle pain, lack of taste or smell, and gastrointestinal symptoms. Participants had to be capable of providing informed consent and self-administering the nasal spray. Exclusion criteria included asymptomatic infection with COVID-19, current tracheostomy or laryngectomy, pregnancy or current lactation, hypersensitivity to the active substance or to any of the excipients, concomitant respiratory therapy (e.g., oxygen or ventilator support) and any clinical signs indicative of moderate, severe or critical COVID-19 symptoms as defined by FDA COVID-19 Guidance Document. Written consent was obtained from all participants.

### Randomization and Masking

80 eligible participants were randomized in a 1:1 ratio to receive the NONS or an identically packaged placebo saline nasal spray. The original study was intended for 50 participants though this expanded in view of not all participants completing symptom data. The study was blinded for the trial participants, care providers and outcome assessors. A blinded randomization list was devised that attributed a coded treatment to each randomization number. This list was computer generated using a block identifier, block size, sequence within block treatment. Seeding was used. Protocols were established for emergency unblinding if used. One participant had not followed the protocol correctly so was not included in the analysis.

### Procedures

Participants were supplied with the nasal spray—either NONS or placebo—and instructed to self-administer 4 sprays, 5-6 times per day for a total of 9 days of treatment, with each spray dispensing approximately 120-140 μL of solution. Participants were instructed to blow their nose prior to use, insert the tip of the spray nozzle into the nostril and administer sprays alternating into each nostril whilst inhaling. 9 days of treatment was decided upon as this would represent at least day 13 since symptoms whereby the viral load would be expected to already be reduced in mild infection.

Nasal and throat swabs were taken at presentation for baseline, and treatment commenced on day 1. Participants were already in isolation and advised to ensure using the spray and taking swabs was performed in isolation of others. Participants took self-sampled swabs on days 2, 4 and 6 and these were taken in the morning prior to treatment to avoid interference with the swab results. Quantitative RT-PCR was performed at the Berkshire Surrey Pathology Services Virology laboratory at ASPHFT to determine SARS-CoV-2 RNA levels. SARS-CoV-2 sequencing for variants was performed at the Public Health England Colindale. Participants completed daily self-reporting questionnaires on symptoms, compliance and tolerance of treatment. Follow-up was carried out for a total of 18 days.

### Outcomes

The outcome measure was the difference in SARS-CoV-2 viral load from baseline through day 6 between NONS and control arms, as measured on quantitative RT-PCR. It was originally intended to study the cycle threshold (ct) value and the viral load though as the viral load is derived from the ct value and represents a more reliable measure of quantity then the load was solely analyzed.

Secondary outcome measures were the difference in proportion of participants and time taken to reach unmeasurable levels of viral load between NONS and control arms on days 2, 4 and 6; the proportion of participants requiring hospitalization or A&E visits for COVID-19 related symptoms by 18 days; the difference in symptom scores from baseline through day 6 between NONS and control arms and the rate of adverse events and discontinuation of treatment.

### Statistical Analysis

The analysis utilized a linear mixed effect model for log (10)SARS-CoV-2 RNA. A random (Gaussian) intercept for subject was included to account for multiple observations. Parameter estimation was performed using maximum likelihood (not restricted/REML) and inference was based on a likelihood ratio test of the full model, versus a null model excluding the main and interaction effects involving arm. In addition to this overall test for treatment effect, least-square mean log (10) SARS-CoV-2 RNA estimates for each time point were generated from the full model accompanied by 95% confidence. As a scenario analysis, a second model was constructed using random effects for treatment group. The analysis used R (2020 version), while simple exploratory analyses were accomplished using MedCalc version 19.7.

### Results

From 11^{th} January through to 21^{st} February 2021, a total of 80 patients were randomized to receive either NONS (40 patients) or placebo (40 patients). Of the patients who had been randomized, 79 met the criteria for inclusion in the analysis (39 in the NONS group and 40 in the placebo group). NONS or placebo nasal spray was self-administered in these patients 5-6 times per day. 79 participants recorded that they had successfully utilized the spray and provided all of the swabs. 1 participant recorded that they had not correctly followed the instructions for using the spray so were excluded.

The two trial groups were well balanced regarding risk factors (Table 3A-1). Patients started NONS or placebo at least 4 days after the onset of symptoms. All of the patients had mild symptoms at the time of randomization. The baseline mean SARS-CoV-2 load of ˜2.4×10⁷ RNA copies/mL of all participants corresponded with expectations of a high SARS-CoV-2 RNA burden.

TABLE 3A-1
Characteristics of the Patients at Baseline

| | |
|---|---|
| NONS group | Placebo group |
| (n = 39) | (n = 40) |

| | | |
|---|---|---|
| Age - mean | 44 | 43.9 |

Sex

| | | | | |
|---|---|---|---|---|
| Male | 16 | (41%) | 13 | (32.5%) |
| Female | 23 | (59%) | 27 | (67.5%) |

Ethnicity

| | | | | |
|---|---|---|---|---|
| White | 33 | (84.6%) | 37 | (92.5%) |
| Black African + Caribbean | 1 | (2.6%) | 0 | (0%) |
| South Asian | 2 | (5.1%) | 3 | (7.5%) |
| Mixed | 2 | (5.1%) | 0 | (0%) |
| Other | 1 | (2.6%) | 0 | (0%) |

BMI

| | | | | |
|---|---|---|---|---|
| BMI ≥ 30 | 12 | (30.8%) | 7 | (17.5%) |
| BMI < 30 | 18 | (46.2%) | 26 | (65%) |
| No data | 9 | (23%) | 7 | (17.5%) |

Comorbidities

| | | | | |
|---|---|---|---|---|
| Any comorbidity | 5 | (12.8%) | 4 | (10%) |
| Chronic lung disease | 1 | (2.6%) | 0 | (0%) |
| Chronic liver disease | 0 | (0%) | 0 | (0%) |
| Chronic heart disease | 0 | (0%) | 0 | (0%) |
| Diabetes | 3 | (7.7%) | 2 | (5%) |
| Hypertension | 2 | (5.1%) | 3 | (7.5%) |

Presenting symptoms

| | | | | |
|---|---|---|---|---|
| Dry cough | 22 | (56.4%) | 27 | (67.5%) |
| Fever | 7 | (17.9%) | 16 | (40%) |
| Loss of sense of smell | 9 | (23.1%) | 5 | (12.5%) |
| None of the above | 6 | (15.4%) | 8 | (20%) |

On day 6, the mean difference from patients on NONS to placebo in the decrease from baseline in the mean log viral load was −0.98 (95% CI, −2.04 to −0.08; P=0.07). The difference of NONS from placebo in the decrease from baseline through day 6 was −5.22 (95% confidence interval [CI], −9.14 to −1.30; P=0.01) by an area under the curve analysis (Table 3A-2).

TABLE 3A-2
Change from Baseline in SARS-CoV-2 RNA

| | NONS | Placebo | Difference |
|---|---|---|---|
| Variable | (N = X) | (N = Y) | (95% CI) |
| Mean difference | −1.36 | −0.15 | −1.21 |
| from baseline at | | | (−2.07 to −0.35); |
| day 2 | | | P = 0.01 |
| Mean difference | −2.40 | −1.19 | −1.21 |
| from baseline at | | | (−2.19 to −0.24); |
| day 4 | | | P = 0.02 |
| Mean difference | −3.32 | −2.34 | −0.98 |
| from baseline at | | | (−2.04 to −0.08); |
| day 6 | | | P = 0.07 |
| Mean difference | −10.17 | −4.95 | −5.22 |
| from baseline | | | (−9.14 to −1.30); |
| through day 6 | | | P = 0.01 |
| (area under the | | | |
| curve analysis) | | | |

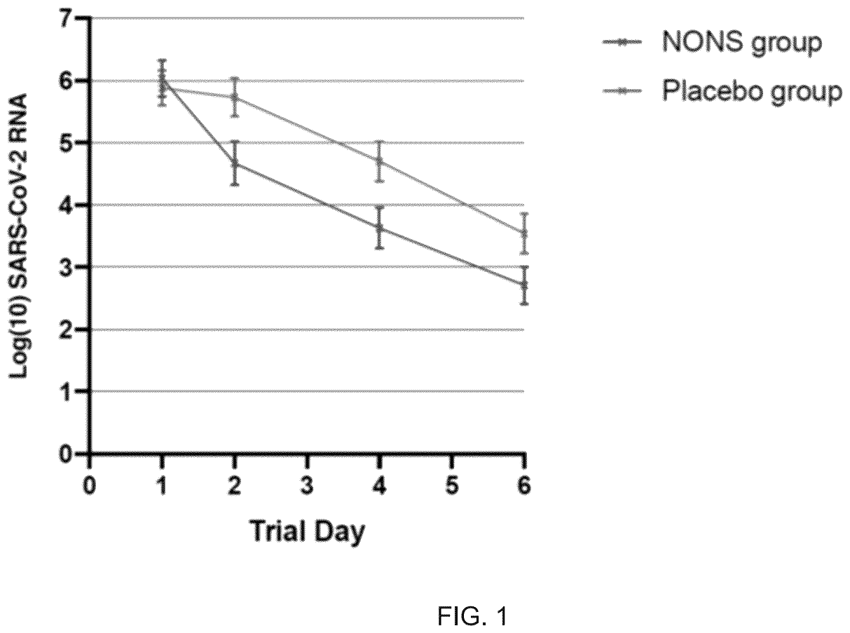

On day 2, the mean difference from patients on NONS to placebo in the decrease from baseline in the mean log viral load was −1.21 (95% CI, −2.07 to −0.35; P=0.01) (Table 3A-2). On day 4, the mean difference from patients on NONS to placebo in the decrease from baseline in the mean log viral load was −1.21 (95% CI, −2.19 to −0.24; P=0.02). Consequently, the mean SARS-CoV-2 RNA concentration was lower on NONS by a factor of 16.2 on days 2 and 4, as illustrated in FIG. 1.

Results were available for 48/79 patients in the per-protocol population on day 1, declining to 32/79 patients on day 9. Assessment of the difference between day 9 score vs day 0 score showed a mean reduction in score for NONS of −5.50 and for control of −4.38 (p=0.495). The time to achieving a sustained symptom score of zero was 7.74 for NONS versus 8.35 for placebo (p=0.48). The time to achieving a sustained symptom score of less than 3 was 5.15 for NONS versus 6.22 for placebo (p=0.65).

One patient in each of the NONS and placebo group sought care due to COVID-19. The patient in the NONS group seeking care was aged 37 years old, had a medical history of asthma and did not have hospitalization beyond 24 hours. A patient in the placebo group had emergency hospitalization and was aged 65 years old. Insufficient numbers of patients completed the symptom scores for statistical analysis.

There were no serious adverse events in patients within the NONS or placebo groups. All viral samples were sequenced for the presence of known SARS-CoV-2 variants of concern. 34 (87.2%) of the NONS group were determined to be lineage B.1.1.7 (VOC202012/01) and the remainder were not determined to be a variant of concern. 34 (85%) of the placebo group were determined to be B.1.1.7 and the remainder were not determined to be a known variant of concern. A PHE matched cohort analysis has reported a death risk ratio for VOC-infected individuals compared to non-VOC of 1.65 (95% CI 1.21-2.25). It is therefore a possibility VOC B.1.1.7 infection is associated with an increased risk of mortality compared to infection with non-VOC viruses.

### Discussion:

In this analysis of the NONS trial, treatment with NONS was found to be effective in reducing the viral load in patients with mild, symptomatic COVID-19 infection. The trial was designed to enroll patients with a recent disease onset to evaluate the effect of early intervention with NONS on the SARS-CoV-2 RNA load in nose and throat swabs. Among the patients who received NONS, the viral load at days 2 and 4 were lower by a factor of 16.2 than that in the placebo group. The clinical interpretation of viral loads to determine whether a virological response has occurred often considers whether there is a 1.0 log change representing a 10 times difference which was exceeded in this study. The difference was less marked at day 6 though this corresponded with a point where the viral load was substantially reduced from baseline for the majority of patients, including those in the placebo group. This correlates with at least day 10 since symptom onset by this point and the reduced load is consistent with the natural course of infection. In addition, the symptom resolution was faster for NONS than placebo in this study though more symptom data would be needed to achieve statistical significance.

It is envisaged that an agent with antiviral and viricidal properties towards SARS-CoV-2 should shorten or prevent the course of disease, reduce immune mediated pathological damage and reduce progression to severe disease. Inhaled NO treatment in severe illness has been observed to improve arterial oxygenation in patients with SARS-CoV beyond termination of NO suggesting a direct effect on the disease. It has also been observed that reducing the transport of viruses towards the acinar airways could provide an opportunity for the prevention of pneumonia. Moreover, a delayed viral clearance for instance can be observed in patients with more severe disease. Ultimately though, the nasopharyngeal viral load has not been validated as a predictor of clinical disease course in COVID-19. Consequently, further study is needed though to fully understand the dynamics of SARS-CoV-2 including the duration of shedding and causal association with clinical progression and severity.

The reduction of viral load is pertinent to antiviral therapy though and in comparison to this study, SARS-CoV-2 neutralizing antibody LY-CoV555 revealed a relative SARS-CoV-2 RNA decrease versus placebo of −0.64 and −0.45 log 10 copies/ml at days 3 and 7 respectively. Furthermore, Oseltamivir is available for influenza virus infection and has been shown to be independently associated with an accelerated decrease in viral RNA concentration −1.19 [0.43] and −0.68 [0.33] log 10 copies/mL for patients treated on day 1 and days 2-3, respectively (P<0.05). The study also observed that undetectable viral RNA levels within 5 days after symptom onset was independently associated with hospital discharge (adjusted hazard ratio, 1.98; 95% CI, 1.34 to 2.93; P=0.001). The observed decreases with NONS at days 2 and 4 in this study exceed the differences with LY-CoV555 and Oseltamivir. A 1-log higher RSV RNA load can be independently associated with a 0.8-day longer hospitalization, respiratory failure and use for intensive care. These observations for influenza and RSV load are not consistent across all studies though this may be due to study limitations including the absence of standardized methods to quantify respiratory viruses.

The lower levels SARS-CoV-2 RNA loads in patients with NONS may also be beneficial in the prevention of SARS-CoV-2 transmission. It has been described that the higher viral loads in patients with SARS-CoV-2 earlier than SARS-CoV may have contributed to the greater difficulty in reducing the onward transmission. Furthermore, it has been observed that the risk of symptomatic COVID-19 was associated with the SARS-CoV-2 RNA levels of contacts and incubation time was shortened in a dose-dependent manner. It is therefore proposed that NONS could reduce the spread of infection by reducing the period of infectivity in contacts. If NONS were also to be used prophylactically, then it could potentially reduce the dose in those exposed. It is envisaged though that the greatest benefit of treatment would be demonstrated by treating as early as possible and potentially before onset of symptoms

Nevertheless, there were no adverse events recorded in the NONS group and no usage problems were described. Uniquely, NONS is portable and can be self-administered at home which would reduce the risk of healthcare associated infections. In addition, the risk of adverse events would be significantly lower with localized application of NONS than by systemic usage of NO at higher dosage requiring specialized gas cylinders and inhalation technologies. Due to the aforementioned rapid and broad effects of NONS against viral, bacterial and fungal pathogens, NONS could be a safe and well tolerated treatment for SARS-CoV-2 and other infections as well. The accelerated clearance of SARS-CoV-2 by NONS could be used to prevent the spread of SARS-CoV-2.

NONS accelerated reduction of SARS-CoV-2 by a mean factor of 16.2 versus placebo at days 2 and 4. Advancing the clearance of SARS-CoV-2 could lead to shorter duration of symptoms and reduced progression to severe infection. The reduction of SARS-CoV-2 could decrease the period of infectivity and prevent transmission. There are interventions recommended for sever SARS-CoV-2 including remdesivir and dexamethasone. However, there are currently no available agents for mild COVID-19 infection so NONS could become a useful treatment for emergency use.

### EXAMPLE 3-B—Synopsis of Clinical Study

### Objectives

The objective was to evaluate the efficacy of NONS compared to placebo to shorten the duration of COVID-19 viral infectivity through day 6 from randomization on treatment (Days 1 to 6).

The secondary objective was to assess the virucidal effect of NONS compared to placebo in the nasal cavity on Days 2, 4 and 6. Additional secondary objectives include an assessment of the efficacy of NONS in the prevention of the progression of COVID-19, an assessment of the reduction in subjects' COVID-19 clinical symptom score, an assessment of the tolerability of NONS in subjects with COVID-19, and the safety of NONS in subjects with COVID-19.

### Methodology

This was a single center, randomized, double-blind, placebo-controlled, Phase IIb study to evaluate the efficacy and safety of nitric oxide nasal spray to reduce the viral load of SARS-CoV-2 in patients with COVID-19 infection.

Potential participants with mild COVID-19 symptoms were approached, briefed and given an information sheet about the trial. If the participant agreed to participate, informed consent was taken. The subject was immediately scheduled to have a SARS-CoV-2 nasal swab rt-PCR and antigen test. If the subject's antigen test was positive, or the subject had previously tested positive for COVID-19 (nasal SARS-CoV-2 rT-PCR within the past 48 hours), a qualified medical staff completed the pertinent medical history and physical examination, including an assessment of flu-like symptoms, and enrolled the participant into the study.

Upon enrollment, subjects were randomized, with a 1:1 NONS treatment to placebo (control) ratio, and given a package containing a 9-day supply of investigational product, nasal spray pump, home nasal swab test kits, shipping envelopes and instructions on how to self-administer the product daily on an outpatient basis (with access to online video directions). Subjects agreed to isolate for the duration of the treatment portion of the study (Days 1 to 9). The total duration of isolation was dictated by the local governments' COVID-19 guidelines and policy.

During the 9 treatment days, each subject administered 2 sprays per nostril using a metered dose nasal spray pump repeated 5-6 times daily while awake, with a cleansing procedure every morning before the first treatment. Subjects collected and entered daily symptom result scores in their diary, including symptom relief medications, through an on-line portal (monitored by study staff for deterioration of condition) to the end of the study (Day 18). Subjects were pre-scheduled to have a nasal swab obtained at their COVID-19 test center or via a home self-test on Days 2, 4 and 6 for viral load analysis. A telephone follow-up occurred on Days 2, 4, 6, 9 and 18. Adverse events, discomfort, pain, discontinuation of treatment, urgent care, emergency room, and hospitalizations were recorded.

### Number of Subjects

Completion of 50 total subjects, 25 per treatment group (NONS and placebo) was initially planned. The trial continued after an informal interim analysis performed on the first 50 subjects suggested NONS treatment was better than placebo (treatment difference p=0.088) and no safety concerns were observed. The revised sample size was increased to 45-50 subjects per group. The study was terminated after approximately 40 subjects per group had been enrolled.

Eighty-three subjects, 53 females and 30 males were enrolled. Forty subjects were randomized to NONS and 40 subjects to placebo. Seventy-nine subjects completed the study with no subject terminating early unrelated to an adverse event. One NONS subject withdrew consent before receiving treatment. Efficacy and safety analyses were performed on 79 subjects (39 NONS and 40 placebo) having data post-baseline.

### Diagnosis and Main Criteria for Inclusion

All subjects meet the following inclusion criteria to participate in the study:
Capable of understanding and providing signed informed consent; ability to adhere to the protocol;
Men and women 18 to 70 years of age;
Internet access; capable plus willingness to use the access to participate in audio or audio/video engagements with medical professionals, receive texts, emails, and phone calls from study staff and had a device with reasonable cellular data or other internet access to submit daily study information using a smart phone, tablet, laptop, or desktop computer during the study period;
COVID-19 infection confirmed with a laboratory SARS-CoV-2 RT-PCR nasal swab;
Specimen (nasal) collected within the past 48 hours;
Mild COVID/FLU symptoms which may include fever, cough, sore throat, malaise, headache, muscle pain, gastrointestinal symptoms, lack of taste or smell without shortness of breath or dyspnea, or no symptoms.

In order to participate subjects do not meet any of the following exclusion criteria:
Not fit to consent and unable to follow the protocol;
Men and women age >70 years;
Current tracheostomy or laryngectomy;
Concomitant respiratory therapy such as oxygen or ventilator support (however, positive airway pressure for obstructive sleep apnea was permitted if treatment was established with good compliance at least 3 months before enrollment);
Need for hospitalization for any reason;
Inability to safely self-administer the nasal spray;
Any clinical contraindications, as judged by the qualified medical practitioner;
Clinical signs indicative of moderate, severe, or critical COVID severity symptoms (as defined by FDA COVID-19 Guidance Document);
Mentally or neurologically disabled patients who were considered not fit to consent to their participation in the study;
Lactating, pregnant or planning to become pregnant during the study period;
Diagnosed with prior COVID-19 infection (i.e., more than 48 hours from the time the test was reported prior to the time of screening).

### Test Product, Dose, and Mode of Administration

The investigational medicinal product (IMP) for nasal irrigation, i.e., NONS was provided in two, 5 mL tubes which was added to the nasal spray bottle (10 mL) solution and replaced every 3 days with fresh solution. The IMP contained sufficient NaCl to create an isotonic (0.9% NaCl) saline solution.

Nitric Oxide Nasal Spray (NONS) was self-administered daily by the subjects throughout each day for 9 continuous treatment days. A cleansing procedure was performed every morning before the first treatment (two sprays, then nose blown after 30 seconds to clear mucous/debris from the epithelial cell surfaces). On treatment Days 1 through 9, each subject administered 2 sprays per nostril (2×140 L=240 μL/nostril; 480 μL for both nostrils) per treatment using the provided metered dose nasal spray pump containing either the NORS formulation or the isotonic saline placebo solution. Each treatment was repeated 5-6 times daily while awake.

Each subject was encouraged to blow their nose prior to each treatment to clear potential mucous debris. The tip of the spray nozzle was placed just barely into the nostril. Sprays were administered into the nostrils, alternating sprays into the lateral nostrils and then straight into the nasal cavities while inhaling. The maximum total exposure per day was 3.36 mL and 30.2 mL over the 54 maximum treatments (9 days).

### Reference Product, Dose, and Mode of Administration

Matching placebo was provided in two, 5 mL tubes (sodium chloride [NaCl] designed to create an isotonic saline solution), which was added to the nasal spray bottle (10 mL) and replaced every 3 days with fresh solution. Placebo nasal spray was administered in the same dose and fashion as active treatment.

### Duration of Treatment

Each subject participated in the study for up to 20 days, including a screening period of up to 2 days (Day −2 to Day 0), 9 continuous treatment days (Day 1 to Day 9), and 8 days for follow up (Day 10 to Day 18). Over the course of the study, 41.7% of the NONS doses and 46.3% of placebo doses were recorded as having been administered; 4.3% of the NONS doses and 4.8% of placebo doses were recorded as having been missed.

### Criteria for Evaluation

One efficacy variable and endpoint for this study is difference in SARS-CoV-2 viral load (Cycle threshold [Ct]) from baseline through Day 6 between NONS and control arms. The Ct value is inversely related to the viral load and for every 3.3 increase in the Ct value reflects a 10-fold reduction in COVID-19 starting material.

A secondary efficacy variable assessment included multiple evaluations. An assessment of the virucidal effect of NONS was compared to placebo in the nasal cavity on Days 2, 4, and 6. An assessment of the proportion of subjects requiring hospitalization or an emergency department visit(s) for COVID-19 flu-like symptoms by Day 18. An assessment of efficacy in the reduction of COVID-19 subject clinical symptom (modified Jackson) scores between NONS and placebo control at Days 2, 4, 6, 9 and 18.

### Safety

Safety included both a tolerability and general safety assessment of NONS compared to placebo in COVID-19 subjects.

### Statistical Methods

Descriptive statistics for continuous monitoring data include the number of subjects with data to be summarized (n), mean, standard deviation and median. All categorical and qualitative data are presented using counts and percentages. Summary statistics were presented by treatment group and overall, unless stated otherwise. Baseline was defined as the last non-missing value before the dose of study drug.

The efficacy analysis was conducted using the intent-to-treat (ITT) population and utilized a linear mixed effect model for Ct with fixed effects for randomized treatment group (NONS, control), age (continuous), presence of comorbidities at randomization (no/yes), and study day (0, 2, 4, 6) was included in the model along with an interaction between treatment group and study day. A random (Gaussian) intercept for subject was included to account for multiple observations. Parameter estimation were performed using maximum likelihood (not restricted/REML) and inference was based on a likelihood ratio test of the full model (above) versus a model excluding the main and interaction effects involving arm.

In addition to this overall test for treatment effect, least-square mean Ct estimates for each timepoint were generated from the full model accompanied by 95% confidence estimates utilizing the sandwich variance estimator. If there was convergence errors for the full model, the comorbidity variable was omitted. The analysis was repeated for the PP population. Estimates of mean Ct by center and time point were also calculated.

All analyses of secondary efficacy endpoints were carried out on the ITT and PP analysis populations, unless otherwise stated, as follows:
1. Mean SARS-CoV-2 (log₁₀) Viral Load Change from Baseline at Day 2, Day 4 and Day 6 (ITT Population)—Mean difference in the (log)viral load over the first 6 continuous days of treatment was compared between treatment groups using a repeated measures t-test. Separate analyses were performed for the change in (log₁₀) viral load at Day 2, Day 4 and Day 6 compared to Baseline for the NONS and Placebo groups.
2. Proportion of Subjects Achieving SARS-CoV-2 (log₁₀) Viral Load Reduction Below Threshold at Day 2, Day 4 and Day 6—Proportion of subjects reaching Ct threshold, i.e., unmeasurable (log)viral load, were compared between the treatment groups using a logistic regression model with fixed effects for randomized group, age, and baseline comorbidity (yes/no). Separate models were fitted for Days 2, 4, and 6. Imputation as in the analysis of the endpoint were followed for this analysis. Rates and 95% CIs were calculated using least-square means.
3. Time to SARS-CoV-2 (log₁₀) Viral Load Reduction Below a Range of Thresholds (1, 2 and 3) at Day 2, Day 4 and Day 6—Time to Ct threshold (unmeasurable viral load) was modeled using a Cox proportional hazards model including fixed effects for treatment group, age, and baseline co-morbidity. Individuals who did not achieve threshold were considered censored at the last available Ct measurement time. Median time and 95% confidence intervals were calculated.
4. Proportion of subjects requiring hospitalization or ED/ER visits by day 18—Subjects were to be modeled using a logistic regression with fixed effects as in the analysis (group, age, co-morbidity) for this assessment. Point estimates and 95% confidence intervals were to be provided. Subjects for whom hospitalization or ER/ED visits cannot be definitively determined for day 18 were to be imputed as having a visit.
5. Modified Jackson Scores Modeled on the Endpoint Analysis (ITT)—Modified Jackson scores were modeled as in the analysis of the endpoint, but all days from randomization through Day 6 were used each with an indicator for whether on or prior to treatment. Symptom scores on the day treatment was initiated were considered prior to treatment unless times of NONS administration and questionnaire completion indicated the opposite. Scores that were missing due to hospitalization, or death, were imputed with the maximum possible score. If scores were missing and a subject subsequently died or was hospitalized prior to Day 18, they were also imputed with the maximum possible score. All other missingness were treated as missing at random and all available data for the subject included in the model.
6. Proportion of Subjects Experiencing Modified Jackson Scores Change from Baseline ≥5 or Reduction to Zero at Days 2, 4, 6, 9 and 18—Proportion of subjects experiencing modified Jackson score (or COVID-19 PRO Score) change of ≥5 from baseline or reduction to zero were modeled using a logistic regression as described for other secondary endpoints at Day 2, 4, 6, 9, and 18. Scores that were missing due to hospitalization, or death, were imputed with the maximum possible score. Scores that were missing and a subject subsequently died or was hospitalized were also imputed with the maximum possible score. All other missingness were treated as missing at random.

All safety analyses were performed on the safety population. The proportion of subjects who discontinued NONS or control treatment on-treatment (Days 1 to 9) were estimated and compared using a Fischer's Exact test (if available). Severity and frequency of adverse events as well as clinically significant changes in oximetry and symptoms (if available) were summarized and presented by arm and period (on-treatment [Days 1 to 9], post-treatment [Days 10 to 18]).

### Results and Conclusions

One objective was to demonstrate the efficacy of NONS to shorten COVID-19 infectivity duration in mild COVID-19 patients. Fifty-four (54) nasal spray treatments were administered 6 times per day for 9 continuous days.

Each treatment considered of 4 sprays (560 μL total), 2 sprays per nostril, for a total dosage regimen of approximately 30 mL. The mean baseline SARS-CoV-2 viral load was 2.415×10¹³ RNA copies per cm³ (mL) across all study subjects.

### Efficacy Results

Objective was to demonstrate the efficacy of NONS to shorten COVID-19 infectivity duration in mild COVID-19 patients. Fifty-four (54) nasal spray treatments were administered 6 times per day for 9 continuous days.

Each treatment considered of 4 sprays (560 μL total), 2 sprays per nostril, for a total dosage regimen of approximately 30 mL. The mean baseline SARS-CoV-2 viral load was 2.415×10¹³ RNA copies per cm³ (mL) across all study subjects.

The efficacy variable was the change from baseline reduction through Day 6 of treatment in ‘Cycle Threshold’, i.e., the difference in SARS-CoV-2 viral load.

The efficacy endpoint for this trial was the comparison of the mean (log)viral load reduction change from baseline between treatment groups. The analysis performed utilized a generalized linear mixed effect model for (log₁₀) viral load with fixed effects for randomized treatment group (NONS, control), age (continuous), presence of comorbidities at randomization, study day (0, 2, 4, 6), and interaction between treatment group and each study day.

SARS-CoV-2 (log)viral load was significantly decreased over the first 6 days of NONS treatment compared to placebo: The treatment difference between NONS and placebo was statistically significant for all three days (p<0.05 at Day 2, 4 and 6 for NONS treatments compared to placebo). Viral load observed in subjects randomized to placebo was significantly higher over the first 6 days compared to those allocated to NONS active treatment.

Impact of the main covariate (treatment group) on viral load over time was assessed by the null model excluding the main covariate and the interaction effect between the main covariate and the study day. The full model and the null model were compared using a likelihood ratio test. The likelihood ratio test for the full model was statistically significantly different from the null model (p=0.01). The combination of the assessment day and use of active treatment significantly predicted viral load level. The null hypothesis is therefore excluded.

The mean treatment difference of the (log)viral load change from baseline to Days 2, 4 and 6 using the area under curve (AUC) assessment from Baseline to Days 2, 4 and 6 was statistically significant favoring NONS over placebo (−5.220; 95% CI=−9.136 to −1.305; p=0.01).

A rapid reduction (95%) in the high SARS-CoV-2 viral load was observed within 24 hours, and a 99% reduction within 72 hours, with NONS treatment. The efficacy results for the per protocol (PP) population are comparable to the ITT population results.

Sensitivity analyses was repeated utilizing a generalized linear mixed effect model for (log)viral load with random effects for randomized treatment group which confirmed the analyses.

### Secondary Efficacy Results

Secondary efficacy endpoints analyzed for the most part broadly support the efficacy of NONS administration in mild COVID-19 patients.
1. Mean SARS-CoV-2 (log)Viral Load Change from Baseline at Day 2, Day 4 and Day 6 (ITT Population)—Change in baseline reduction in SARS-CoV-2 (log)viral load were statistically significant at Day 2 (p=0.008) and Day 4 (p=0.021) on NONS treatments compared to placebo. The CFB reduction in SARS-CoV-2 (log)viral load was greater, but not statistically significant at Day 6 (p=0.094) on NONS treatments compared to placebo. Overall, these results are consistent with the result outcomes. Similar results were observed for the PP population. All viral samples were sequenced for the presence of known SARS-CoV-2 variants of concern (VOC). Thirty-four (87.2%) of the NONS group subjects were determined to have the B.1.1.7 lineage (VOC202012/01) with the remainder determined not to be VOC. Thirty-four (85.0%) of the placebo group subjects were determined to be B.1.1.7 lineage with the remainder determined not to be a known variant of concern.
2. Proportion of Subjects Achieving SARS-CoV-2 (log)Viral Load Reduction Below Threshold at Day 2, Day 4 and Day 6—No statistically significant difference between NONS treatment and placebo at any of the three threshold values were established, with the exception of the day 4 analysis for the (log) viral load threshold of 3 (p=0.012). A numerical benefit for NONS treatment was observed in all analyses, with the exception of day 2 analysis for the log viral load threshold of 1. The study was likely underpowered to demonstrate a consistent statistically significant impact for this secondary endpoint. Similar results were observed for the PP population.
3. Time to SARS-CoV-2 (log)Viral Load Reduction Below a Range of Thresholds (1, 2 and 3) at Day 2, Day 4 and Day 6—None of the differences between the NONS treatments and placebo at each of the thresholds were statistically significant. The threshold 3 median time was the same for both the NONS treatment and placebo (6 hours), while the mean time was 4 hours on NONS treatment and 6 hours for placebo. No additional analyses were conducted on the PP population.
4. Proportion of subjects requiring hospitalization or ED/ER visits by day 18—2 subjects had hospitalization (one in each treatment group) which were considered non-treatment related and not adverse events per the investigator. No additional ED/ER visits were used by patients subsequent to their recovery. No statistical analyses were carried out for this outcome on the ITT or PP populations.
5. Modified Jackson Scores Modeled on the Endpoint Analysis (ITT)—Complete data on modified Jackson symptom scores were available for 48 of the 79 subjects in the PP population on Day 1, declining to 32 of the 79 subjects on Day 9. The statistically analysis for the aggregate total Modified Jackson symptom scores on each of the 9 days demonstrated no statistically significant difference between treatments for any day.
6. Proportion of Subjects Experiencing Modified Jackson Scores Change from Baseline ≥5 or Reduction to Zero at Days 2, 4, 6, 9 and 18—No analysis was undertaken for the modified Jackson symptom scores change from baseline of greater than or equal to 5 for either the ITT or PP populations.

The time-to-event analysis to achieve a sustained symptom score of zero was limited in that there were 11 subjects overall that achieved this endpoint. Due to this small sample, a second analysis based on the achievement of a sustained symptom score of <3 was performed which yielded 32 subjects. In both cases, the Kaplan-Meier curves showed an apparent benefit for NONS, although not statistically significant in either case.

### Safety Results

NONS treatments were well-tolerated and considered safe by the investigator, in that:
No deaths and no life-threatening TEAEs occurred during the study. No subject withdrew from the study due to an AE.
No subjects reported experiencing a TEAE from treatment per the investigator. One subject in each treatment group reported a hospitalization unrelated to treatment.
There were no complications, misuse, abuse or overdose situations with the self-administration of NONS.

Overall, the 54 total nitric oxide nasal spray treatments, administered 6 times per day for 9 continuous days, with each treatment of 4 sprays (560 μL total), delivered as 2 sprays per nostril for a total dosage regimen of approximately 30 mL was well-tolerated in this Phase IIb efficacy and safety study. No new safety concerns were identified with this novel NO therapy (NONS) and its administration to mild COVID-19 patients.

### Conclusion

The nasal cavity is the major route for host entry and infectivity of the COVID-19 virus. An effective COVID-19 antiviral therapy that could shorten or prevent the course of the disease, reduce immune mediated pathological damage and lower disease severity is currently lacking. Nitric oxide has antimicrobial activity against bacteria, yeast, fungi, and viruses both in vitro and in vivo animal studies. NO also prevents the fusion between the SARS-CoV-2 Spike protein and its cognate receptor, ACE-2.

The current study was devised to determine the clinical efficacy of Nitric Oxide Nasal Spray (NONS) for the treatment of mild COVID-19 infection. The goal was to characterize nasal viral concentrations by quantitative real-time reverse-transcriptase polymerase chain reaction and establish the ability of treatment to clear the virus from the nasal cavity. The outcome measure was the difference in SARS-CoV-2 viral concentrations from baseline through Day 6 between NONS treatment and placebo. Eighty adults diagnosed with mild COVID-19 in the community were randomized in this double-blinded, placebo-controlled, phase IIb clinical trial.

NONS treatment started on or before day 5 of COVID-19 symptom onset was independently associated with an accelerated decrease in SARS-CoV-2 RNA concentration of −1.21 and −1.21 log₁₀ copies/mL on days 2 and 4 compared to placebo. Mean SARS-CoV-2 RNA concentration was lower on NONS treatment by a factor of 16.2 at both day 2 and 4. Day 6 equated to at least 10 days since symptom onset when a reduction of SARS-CoV-2 viral load with or without treatment was expected. Nevertheless, the SARS-CoV-2 RNA concentration was still lower on NONS treatment at day 6. The area under the curve analysis revealed a mean difference of −5.22 log₁₀ copies/mL with NONS treatment compared to placebo over the first 6 days of treatment. A rapid reduction (95%) in the high SARS-CoV-2 viral load occurred within 24 hours, and 99% reduction within 72 hours, on NONS treatments.

The majority of patients in this study were positive for the SARS-CoV-2 variant of concern (VOC202012_01). By comparison, a study of SARS-CoV-2 neutralizing antibody LY-CoV555 revealed a relative SARS-CoV-2 RNA concentration decrease versus placebo of −0.64 and −0.45 log₁₀ copies/mL at days 3 and 7, respectively. Oseltamivir has been shown to be independently associated with an influenza RNA concentration decrease of −1.19 and −0.68 log₁₀ copies/mL on days 2 and 4, respectively.

The reduction of SARS-CoV-2 viral load reduction in COVID-19 infected patients strongly correlates with better clinical outcomes, with viral load kinetics and duration of viral shedding being some determinants of disease transmission. Outcomes are best if viral load reduction begins within the first 5 days of symptom onset resulting in the lack of persistence of live viruses after 8-9 days, as demonstrated in monoclonal antibodies studies and vaccine trials using measured nasal viral load techniques comparable to the current trial.

Nasal deliver of NONS is effective in reducing COVID-19 viral load as are systemic therapies under investigation or currently being used to treat viral infections. Nitric oxide therapy is often overlooked as a COVID-19 treatment when potential beneficial treatments and pathogenic new insights are reviewed.

Overall, a difference in treatment across the efficacy and multiple secondary efficacy endpoints was observed between NONS compared to placebo. SARS-CoV-2 viral concentrations were immediately and consistently lowered with NONS treatment by a factor of 16.2-fold, suggesting a shortened COVID-19 infectivity duration in mild COVID-19 patients.

NONS could be used across the population for prevention or early treatment if new variants reduce the efficacy of the current vaccines. NONS could also provide antiviral treatment to those infected who may not yet have been fully vaccinated, those who are unable to be vaccinated or those with infection despite vaccination.

### Efficacy

The objective was to demonstrate the efficacy of NONS to shorten COVID-19 infectivity duration in mild COVID-19 patients (ITT Population). The efficacy variable was the change from baseline reduction through Day 6 of treatment in ‘Cycle Threshold’, i.e., the difference in SARS-CoV-2 viral load. The analysis performed utilized a linear mixed effect model for (log₁₀) viral load with fixed effects for the randomized treatment groups. The mean baseline SARS-CoV-2 viral load was 2.415×10¹³ RNA copies per cm³ (mL) across all study subjects.

SARS-CoV-2 (log)Viral Load Reduction: Viral load was significantly decreased over the first 6 days of treatment with the NO Nasal Spray Treatments compared to Placebo. The treatment difference was statistically significant for Day 2 (p=0.006), Day 4 (p=0.007), and Day 6 (p=0.035), respectively. The PP population results were comparable to the ITT population results.

Likelihood Ratio Test: Impact of the main covariate (treatment group) on viral load over time was assessed using the full model compared to a null model, excluding the main covariate and the interaction effect between the main covariate and the study day. The likelihood ratio test suggests that the full model significantly differs from the null model (p=0.01). The combination of the assessment day and use of active treatment significantly predict the viral load level. The null hypothesis is therefore excluded. The PP population results were comparable to the ITT population results.

Area Under the Curve (log)Viral Load Change from Baseline: Mean treatment difference using the area under curve from baseline (CFB) over the first 6 days of NONS treatment was −5.220 with a 95% CI −9.136 to −1.305 (p=0.01). A rapid reduction (95%) in the high SARS-CoV-2 viral load was observed within 24 hours, and a 99% reduction within 72 hours, with NONS treatments. The PP Population results were comparable to the ITT Population results.

SARS-CoV-2 (log)viral load reduction and the likelihood ratio test using sensitivity analyses with random effects for treatment groups was comparable to the fixed effects assessment.

Secondary efficacy analyses for the most part broadly support the efficacy of nitric oxide nasal spray treatment in mild COVID-19 patients:

Mean SARS-CoV-2 (log₁₀) Viral Load CFB at Day 2, Day 4 and Day 6: The change in baseline reduction in SARS-CoV-2 (log)viral load were statistically significant at Day 2 (p=0.008) and Day 4 (p=0.021) with NONS treatments compared to Placebo. The CFB reduction in SARS-CoV-2 (log)viral load was greater, but not statistically significant at Day 6 (p=0.094) on NONS for the ITT population. Similar results were observed for the PP population.

Proportion of Subjects Achieving SARS-CoV-2 (log₁₀) Viral Load Reduction Below Threshold Ranges: No statistically significant difference between NONS treatment and placebo at any of the three threshold values were established, with the exception of the day 4 analysis for the (log) viral load threshold of 3 (p=0.012) for the ITT population. Similar results were observed for the PP population.

Time to SARS-CoV-2 (log₁₀) Viral Load Reduction Below a Range of Thresholds: None of the differences between the NONS treatments and placebo at each of the thresholds were statistically significant for the ITT population. No analyses were conducted on the PP population.

Modified Jackson Scores Modeled on the Endpoint Analysis: There was no statistically significant difference between treatments for any of the 9-day aggregate total scores.

Proportion of Subjects Experiencing Modified Jackson Scores Change from Baseline ≥5 or Reduction to Zero at Days 2, 4, 6 and 9: No statistical analyses were carried on the proportion of subjects experiencing a CFB≥5 or reduction to zero at Days 2, 4, 6, 9 and 18. A time-to-event analysis to achieve a sustained symptom score of zero to Day 9 was performed which showed an apparent benefit for NONS, although not statistically significant.

No statistical analyses were carried out on the ITT and PP populations for the proportion of subjects requiring hospitalization or ED/ER visits.

Overall, a difference in treatment across the efficacy and multiple secondary efficacy endpoints was observed between NONS and placebo. SARS-CoV-2 viral loads were immediately and consistently lower with NONS treatments, suggesting a shortened COVID-19 infectivity duration in mild COVID-19 patients. All viral samples were sequenced for the presence of known SARS-CoV-2 variants of concern (VOC). Thirty-four (87.2%) of the NONS group subjects were determined to have the B.1.1.7 lineage (VOC202012/01) with the remainder determined not to be VOC. Thirty-four (85.0%) of the placebo group subjects were determined to be B.1.1.7 lineage with the remainder determined not to be a known variant of concern.

### Safety

No clinically relevant changes from baseline were evident in NO subjects with continuous safety monitoring procedures instituted during each dose administration, and follow-up procedures.

The 54 total nitric oxide nasal spray treatments, administered 6 times per day for 9 continuous days, with each treatment of 4 sprays (560 μL total volume), delivered as 2 sprays per nostril for a total dosage regimen of approximately 30 mL was efficacious and well-tolerated in this Phase IIb efficacy and safety study. No new safety concerns were identified with this novel NO therapy (NONS) and its nasal administration to mild COVID-19 patients.

### EXAMPLE 3-C—Clinical Study Introduction and Objectives

### Introduction

The dosage regimen of NORS proposed for this study has been tested in vitro against H1N1 and H3N2. NORS eradicated H1N1 and H3N2 within 30-60 seconds of exposure using very high viral titers (>10⁶ PFU/mL). Recent tests confirm NORS inactivates more than 99.9% (to below the limit of detection) of SARS-CoV-2, within two minutes in laboratory tests using recent clinical isolates of titers of >10⁴ PFU/mL.

NORS has been previously administered topically to 21 individuals at the same concentration as the nitric oxide nasal spray (NONS) proposed for this study in a Health Canada approved clinical trial to treat Tenia Pedis. The treatment was tolerated, with no SAEs and a small number of mild adverse events (AEs) reported. In addition, environmental and user safety were evaluated and NORS was deemed to be safe.

In one embodiment, the NORS can be used as a sinus irrigation therapy for treating sinusitis, including individuals with recalcitrant chronic sinusitis (CRS). Initially, a dose escalation study was conducted to identify the maximum tolerated dose for a single daily treatment in subjects with CRS. The study identified the maximum tolerated dose as 4X the current study proposed dose. It was also noted that no severe adverse events were recorded and that all 5 subjects had a significant improvement in their quality of life (measured by SNOT-22) and sinusitis severity (measured by endoscopy evaluation). The dosage range for treating sinusitis can vary, including the dosage ranges based on the NORS compositions recited herein. In one embodiment, the dosage range from a 50 mL sinus irrigation to a 500 mL sinus irrigation. In some embodiments, the dosage range can be a 240 mL sinus irrigation. In some embodiments, the irrigation amount can be from 100 mL to 240 mL and the NORS used can be any specific NORS recited herein.

### Objectives

The objective was to evaluate the efficacy of NONS compared to placebo to shorten the duration of COVID-19 viral infectivity through day 6 from randomization on treatment (Days 1 to 6). The secondary objective was to assess the virucidal effect of NONS compared to placebo in the nasal cavity on Days 2, 4 and 6. Additional secondary objectives include an assessment of the efficacy of NONS in the prevention of the progression of COVID-19, an assessment of the reduction in subjects' COVID-19 clinical symptom score, an assessment of the tolerability of NONS in subjects with COVID-19, and the safety of NONS in subjects with COVID-19.

### EXAMPLE 3-D—Study Design and Plan

### Description:

This was a multicenter, randomized, double-blind, placebo-controlled, Phase IIb study to evaluate the efficacy and safety of nitric oxide nasal spray to reduce the viral load of SARS-CoV-2 in patients with COVID-19 infection.

Potential participants with mild COVID-19 symptoms were approached, briefed and given an information sheet about the trial. If the participant agreed to participate, informed consent was taken. The subject was immediately scheduled to have a SARS-CoV-2 nasal swab rt-PCR and antigen test. If the subject's antigen test was positive, or the subject had previously tested positive for COVID-19 (nasal SARS-CoV-2 rT-PCR within the past 48 hours), a qualified medical staff completed the pertinent medical history and physical examination, including an assessment of flu-like symptoms, and enrolled the participant into the study.

Upon enrollment, subjects were randomized, with a 1:1 NONS treatment to placebo (control) ratio, and given a package containing a 9-day supply of investigational product, nasal spray pump, home nasal swab test kits, shipping envelopes and instructions on how to self-administer the product daily on an outpatient basis (with access to online video directions). Subjects agreed to isolate for the duration of the treatment portion of the study (Days 1 to 9). The total duration of isolation was dictated by the local governments' COVID-19 guidelines and policy.

During the 9 treatment days, each subject administered 2 sprays per nostril using a metered dose nasal spray pump repeated 5-6 times daily while awake, with a cleansing procedure every morning before the first treatment. Subjects collected and entered daily symptom result scores in their diary, including symptom relief medications, through an on-line portal (monitored by study staff for deterioration of condition) to the end of the study (Day 18). Subjects were pre-scheduled to have a nasal swab obtained at their COVID-19 test center or via a home self-test on Days 2, 4 and 6 for viral load analysis. A telephone follow-up occurred on Days 2, 4, 6, 9 and 18. Adverse events, discomfort, pain, discontinuation of treatment, urgent care, emergency room, and hospitalizations were recorded.

### Discussion:

Potential control subjects were randomly assigned placebo from the targeted study population. During the pretreatment phase, all subjects' baseline data included demographics, medical history and physical examination. Concomitant medications were recorded at each visit. All subjects could continue their usual daily medications, including ACE inhibitors. Intranasal steroids, antihistamines, anticholinergics and migraine therapy could be continued, but could not be administered within 1 hour before or after treatments.

The Daily Treatment Questionnaire recorded treatment compliance, tolerance, ease of use, and adverse events. A discomfort/pain scale was included, and used. A Patient Reported Outcome (PRO) COVID Symptoms Questionnaire (e-form) was used which combined symptom items from the Common Terminology Criteria for Adverse Event (CTCAE) with the Modified Jackson Cold Score for acute upper respiratory tract infections to capture COVID-19 related symptoms. Twelve relevant symptoms were scored based on a scale of 0-3, i.e., 0=absent; 1=mild; 2=moderate; 3=severe for a maximum score of 36 for the worst most symptoms.

A PRO EuroQol five dimensions questionnaire (EQ5D5L) was used which defines health in terms of 5 dimensions: Mobility, Self-Care, Usual Activities, Pain/Discomfort, and Anxiety/Depression. Each dimension has 5 levels of response categories corresponding to no problems, slight problems, moderate problems, severe problems and extreme problems. The instrument is designed for self-completion. Subjects rated their overall health at the end of the questionnaire on a 0-100 vertical (hash-marked), visual analogue scale.

The follow-up telephone calls conducted by the study staff requested subjects to confirm their current health status including the presence of any COVID-19 symptoms such as fever, cough, dyspnea, sneezing, ageusia, anosmia, headache, major fatigue, loss of appetite, general muscle pain, diarrhea, sore throat and loss of smell/taste. Subjects were reminded about their daily treatment regimen and were asked about any challenges with the administration or any adverse events they may have experienced. Follow-up questionnaires were emailed on Days 9 and 18, which took 2-5 minutes to complete. Total participation time was up to 19 days (1 day for screening [Day 0], 9 days on treatment [Days 1-9], and 9 days of follow-up [Days 10-19]).

### Selection of Study Population:

Inclusion: All subjects meet the following inclusion criteria to participate in the study:
Capable of understanding and providing signed informed consent; ability to adhere to the protocol;
Men and women 18 to 70 years of age;
Internet access; capable plus willingness to use the access to participate in audio or audio/video engagements with medical professionals, receive texts, emails, and phone calls from study staff and had a device with reasonable cellular data or other internet access to submit daily study information using a smart phone, tablet, laptop, or desktop computer during the study period;
COVID-19 infection confirmed with a laboratory SARS-CoV-2 RT-PCR nasal swab;
Specimen (nasal) collected within the past 48 hours;
Mild COVID/FLU symptoms which may include fever, cough, sore throat, malaise, headache, muscle pain, gastrointestinal symptoms, lack of taste or smell without shortness of breath or dyspnea, or no symptoms.

Exclusion: In order to participate subjects do not meet any of the following exclusion criteria:
Not fit to consent and unable to follow the protocol;
Men and women age >70 years;
Current tracheostomy or laryngectomy;
Concomitant respiratory therapy such as oxygen or ventilator support (however, positive airway pressure for obstructive sleep apnea was permitted if treatment was established with good compliance at least 3 months before enrollment);
Need for hospitalization for any reason;
Inability to safely self-administer the nasal spray;
Any clinical contraindications, as judged by the qualified medical practitioner;
Clinical signs indicative of moderate, severe, or critical COVID severity symptoms (as defined by FDA COVID-19 Guidance Document);
Mentally or neurologically disabled patients who were considered not fit to consent to their participation in the study;
Lactating, pregnant or planning to become pregnant during the study period;
Diagnosed with prior COVID-19 infection (i.e., more than 48 hours from the time the test was reported prior to the time of screening).

### Removal

Withdrawal criteria included that any participant could withdraw from the trial at any time without giving a reason.

### EXAMPLE 3-E—Treatments

The nitric oxide nasal spray (NONS) and placebo solutions were delivered to individuals every three days over the nine days of treatment.

The investigational medicinal product (IMP) for nasal irrigation, i.e., NONS was provided in two, 5 mL tubes which was added to the nasal spray bottle (10 mL) solution and replaced every 3 days with fresh solution. Placebo was provided in two, 5 mL tubes (sodium chloride [NaCl] designed to create an isotonic saline solution), which was added to the nasal spray bottle (10 mL) and replaced every 3 days with fresh solution. The IMP contained sufficient NaCl to create an isotonic (0.9% NaCl) saline solution, as depicted in Table 3E-1.

TABLE 3E-1
Description of Investigational Medicinal Products

| | Treatment | Comparator |
|---|---|---|
| Intervention | Nitric Oxide (NONS-20 | Placebo (Saline-Nasal |
| Name: | Nasal Spray) | Spray) |
| Dosage | NORS, liquid 20 mM | Matching placebo solution |
| Formulation: | sodium nitrite at | in a manual nasal spray |
| | pH 3.5 (NO 0.22 ppm) | pump container. Each 3 |
| | solution in a manual | days the nasal spray |
| | nasal spray pump | bottle was comprised of |
| | container. Each 3 | two 5 mL liquid tubes |
| | days the nasal spray | (C and D). Tube C |
| | bottle was comprised | contained 5 mL of saline. |
| | of two 5 mL liquid | Tube D contained 5 mL of |
| | tubes (A and B). Tube | saline. Content of tubes |
| | A contained 5 mL of | C and D were emptied into |
| | saline and 13.8 mg of | the nasal spray bottle |
| | sodium nitrite. Tube | every 3 days. |
| | B contained 5 mL of | |
| | saline and 11.5 mg | |
| | of citric acid. | |
| | Content of tubes A | |
| | and B were emptied | |
| | into the nasal spray | |
| | bottle every 3 days. | |

The NONS Package Kit contained 3 tubes labelled A, 3 tubes labelled B, 1 empty nasal spray bottle, and labelled secondary packaging. The Placebo package Kit containing 3 tubes labelled C, 3 tubes labelled D, 1 empty nasal spray bottle, and labelled secondary packaging. Active investigational product and placebo were delivered in identical packaging with unique identification codes for each package.

Enrolled subjects were randomized to NONS treatment or the placebo group. Randomization was performed prior to study treatment and was based on a computer-generated list via the study eCRF database.

The objective was to provide a NO gas formulation equivalent using a nitric oxide releasing solution (NORS) without the need for gas cylinders or high pressure. The benefit of NORS includes its ability to release the fast-acting viricidal dose of NO for a sustained period of time (at least 5 minutes), at the target site (nasal mucosa/lungs), while producing insignificant oxidative insult to respiratory epithelia, and minimal systemic methemoglobin levels.

Nitric Oxide Nasal Spray (NONS) was self-administered daily, by the participant throughout each day, for 9 continuous treatment days. A cleansing procedure was performed every morning before the first treatment (two sprays, then nose blown after 30 seconds to clear mucous/debris from the epithelial cell surfaces). Video instructions for the preparation and use of the spray pump were available online for subjects.

On treatment Days 1 through 9, each subject administered 2 sprays per nostril (2×140 μL=240 μL/nostril; 480 μL for both nostrils) per treatment using the provided metered dose nasal spray pump containing either the NORS formulation or the isotonic saline placebo solution. Each treatment was repeated 5-6 times daily while awake.

Each subject was encouraged to blow their nose prior to each treatment to clear potential mucous debris. The tip of the spray nozzle was placed just barely into the nostril. Sprays were administered into the nostrils, alternating sprays into the lateral nostrils and then straight into the nasal cavities while inhaling. The maximum total exposure per day was 3.36 mL and 30.2 mL over the 54 maximum treatments (9 days).

### Prior and Concomitant Therapy:

Concomitant medications were recorded at each visit. All subjects could continue their usual daily medications, including ACE inhibitors. Intranasal steroids, antihistamines and anticholinergics could be continued. Intranasal rescue medications could be used as prescribed for migraine, etc. Prescribed nasal sprays could not be used less than one hour before or one hour after each treatment. Asthma inhalers could be taken as previously prescribed and as needed.

Acetaminophen, naproxen sodium and ibuprofen could be used for pain and fever according to package labelling BUT needed to be linked to symptoms and recorded in the Daily Treatment Questionnaire. Guaifenesin and dextromethorphan could be used orally for cough according to package labelling. Pseudoephedrine could be used orally for nasal congestion.

The medications listed in Table 3E-1 were excluded or prohibited within the times indicated before or during the execution of the study. Any use of a prohibited medication was considered a protocol violation.

TABLE 3E-1
Excluded or Prohibited Medications

| | Prohibited Time with |
|---|---|
| Excluded Medications | Respect to Procedures |
| Oxymetazoline (Afrin) | Day 0 to Day 18, or |
| | end of the Study |
| Xymetazoline | Day 0 to Day 18, or |
| | end of the Study |
| Phenylephrine | Day 0 to Day 18, or |
| (Neosynephrine) | end of the Study |

Subjects requiring initiation of new medications, or treatment for COVID-19 symptoms, at any point contacted the study coordinator at the central site. Medications used for COVID-19 symptoms were recorded for each use for a specific symptom and reported in the Daily Treatment Questionnaire.

### Treatment Compliance:

Study personnel monitored compliance by reviewing study medication dispensed in the pharmacy drug accountability log. Subjects were asked to return completed, partially used and unused treatment packs to the trial center. In the follow-up call after 18 days subjects were asked for their (honest) assessment of how much of the investigational treatment had been used and subsequently recorded.

### EXAMPLE 3-F—Efficacy and Safety Assessment

All efficacy and safety assessments performed in this study are listed Table 3F-1. The objective of the trial was to shorten the duration of COVID-19 infectivity on treatment Day 1 to Day 6 using NONS. To determine if NONS could treat mild COVID-19 infection, the endpoint needed to demonstrate that the viral load decreased in a shorter period for those subjects being treated with NONS than those in the control arm.

However, live COVID-19 virus is often isolable during the first week of symptoms, even with positive SARS-CoV-2 reverse transcriptase quantitative PCR (RT-qPCR) tests. Laboratories are difficult to find with the capability and bandwidth to support research studies that used repeated SARS-CoV-2 viral culture support, let alone perform actual live viral loads. Currently, diagnosis and surveillance depends on RT-qPCR testing, but these results are binary and are reported as positive or negative, and do not provide a measure of viral load.

Viral load for this study was measured as “cycle threshold”, reported as a whole number to quantitate changes in viral load from nasal swabs without the need for viral cultures. The cycle threshold (Ct) correlates inversely to live virus and viral load and was used in this study as the endpoint measure to determine the antiviral effectiveness of NONS.

The secondary objectives were to assess the degree of NONS virucidal effect, prevention of COVID-19 progression, reduction of COVID-19 symptoms, and assess to the tolerability and safety of NONS in subjects with COVID-19.

TABLE 3F-2
Study Schedule for Screening, Qualification (Baseline), Procedures and Follow-up

| | | | | | Follow Up | |
|---|---|---|---|---|---|---|
| | | Treatment | | | End | |
| | | | Days 2, | | of | Early |
| | Screening | Daily | 4 &6 | | Study | Termination |
| | Baseline | Days | (for | Days | Days | Days |
| Study Day | Day 0 | 1-9 | swabs) | 9 ± 2 | 18 ± 2 | 1 to 9 |
| Phone Call/Visit | X | X | X | X | X | X |
| Inclusion & Exclusion | X | | | | | |
| Criteria | | | | | | |
| Subject Informed Consent | X | | | | | |
| Demographics | X | | | | | |
| Medical History | X | | | | | |
| (Telehealth & Self- | | | | | | |
| Reporting) | | | | | | |
| Physical Exam (If possible/ | X | | | | X | X |
| Information collected | | | | | | |
| Concomitant Medications | X | | | | X | X |
| Randomization | X | | | | | |
| COVID Related Symptoms | | | | X | X | |
| Captured | | | | | | |
| NORS Self Administration | | X | | | | |
| (Video link instruction) | | | | | | |
| Nasal Swab (Self- | X | | X | | | |
| collecting) Viral Load | | | | | | |
| Patient Reported Outcome | | X | | | | |
| COVID Symptoms | | | | | | |
| (Modified Jackson | | | | | | |
| Questionnaire) | | | | | | |
| PRO EuroQol 5 | | X | | | | |
| Dimensions Questionnaire | | | | | | |
| (EQ5D5L) | | | | | | |
| Daily Treatment | | X | | | | |
| Questionnaire (Self- | | | | | | |
| Reporting) | | | | | | |
| Follow Up Questionnaire | | | X | X | | |
| Adverse Event Assessment | | | | X | X | X |
| Study Drug Accountability/ | | | | X | | X |
| Compliance | | | | | | |
| Need for Hospitalization | | | | X | X | |

The efficacy variable and endpoint for this study is difference in SARS-CoV-2 viral load (Cycle threshold [Ct]) from baseline through Day 6 between NONS and control arms.

The SARS-CoV-2 RT-qPCR test provides real-time quantification by first reverse transcribing SARS-CoV-2 RNA into DNA (RT operation), and then performing qPCR where a fluorescence signal increases proportionally to the amount of amplified nucleic acid, enabling accurate quantitation of the RNA in the sample. If the fluorescence reaches a specified threshold within a certain number of PCR cycles (Ct value), the sample is considered a positive result. The Ct value is inversely related to the viral load and for every 3.3 increase in the Ct value reflects a 10-fold reduction in COVID-19 starting material. Secondary efficacy variable assessments include multiple endpoints.

An assessment of the virucidal effect of NONS was compared to placebo in the nasal cavity on Days 2, 4, and 6. The three secondary endpoints for this assessment were the difference in the mean SARS-CoV-2 (log)viral load; proportion of subjects reaching Ct threshold, the unmeasurable viral load; and the difference in time to unmeasurable viral load achieved between NONS and placebo, all evaluated at Days 2, 4 and 6 to match the analysis.

Also, an assessment of the efficacy of NONS in prevention of the progression of COVID-19 was performed. The secondary endpoint for this assessment was the proportion of subjects requiring hospitalization or an emergency department visit(s) for COVID-19 flu-like symptoms by Day 18.

An assessment of efficacy in the reduction of COVID-19 subject clinical symptom scores was performed. The two secondary endpoints for this assessment were the difference in modified Jackson score from baseline through Day 6 between NONS and placebo to match the analysis; and the difference in proportion of subjects experiencing modified Jackson score reduction of ≥5 from baseline or a reduction to zero from baseline, between NONS and placebo control at Days 2, 4, 6, 9 and 18.

Safety included both a tolerability and general safety assessment of NONS in COVID-19 subjects. The safety endpoints included:
A tolerability endpoint which assessed AEs for each treatment and discontinuation of NONS in subjects with COVID-19 during treatment Days 1-9.
A safety endpoint which assessed AEs over the entire clinical execution of the study, i.e., Days 1 to 18.

### EXAMPLE 3-G—Efficacy and Safety Analysis

The efficacy analysis was conducted using the intent-to-treat (ITT) population and utilized a linear mixed effect model for Ct with fixed effects for randomized treatment group (NONS, control), age (continuous), presence of comorbidities at randomization (no/yes), and study day (0, 2, 4, 6) was included in the model along with an interaction between treatment group and study day. A random (Gaussian) intercept for subject was included to account for multiple observations. Parameter estimation were performed using maximum likelihood (not restricted/REML) and inference was based on a likelihood ratio test of the full model (above) versus a model excluding the main and interaction effects involving arm. In addition to this overall test for treatment effect, least-square mean Ct estimates for each timepoint were generated from the full model accompanied by 95% confidence estimates utilizing the sandwich variance estimator. If there was convergence errors for the full model, the comorbidity variable was omitted.

To corroborate the findings of the linear model, and to accommodate the possibility of a ceiling effect for Ct, a clustered Cox model was fitted to the data. Individuals with Ct beyond the threshold of 40, were considered right censored at 40. The Cox model utilized the same fixed and random effects as the linear mixed model and the p-value for treatment effect was from a likelihood ratio test, i.e., partial likelihood ratio test of full versus reduced model.

Ct measurements that were missing because of a subject being hospitalized, or dies on or prior to Day 6, was imputed with the lowest Ct (highest viral load) value observed in either arm. If Ct measurements were missing and a subject was subsequently hospitalized, or dies prior to Day 18, they were imputed with the lowest Ct value observed in either arm. Withdrawal of consent, loss-to-follow-up, investigator withdrawal, or other missingness unassociated with COVID-19 progression were assumed missing-at-random and their remaining available scores were used in the analysis. Subjects missing all four Ct measurements (baseline, Days 2, 4, and 6) were imputed with the lowest Ct score observed in either arm for the analysis. If more than 5% of subjects (3 or more subjects) were missing all four Ct results, two sensitivity analyses were to be conducted; 1) imputing mean (within-arm/site/age) strata) scores and 2) multiple imputation.

The analysis was repeated for the PP population. Estimates of mean Ct by center and time point were also calculated. Secondary endpoints were analyzed in the ITT and PP populations.
1. Mean SARS-CoV-2 (log₁₀) Viral Load Change from Baseline at Day 2, Day 4 and Day 6 (ITT Population)—Mean difference in the (log)viral load over the first 6 continuous days of treatment was compared between treatment groups using a repeated measures t-test. Separate analyses were performed for the change in (log)viral load at Day 2, Day 4 and Day 6 compared to Baseline for the NONS and Placebo groups.
2. Proportion of Subjects Achieving SARS-CoV-2 (log₁₀) Viral Load Reduction Below Threshold at Day 2, Day 4 and Day 6—Proportion of subjects reaching Ct threshold, i.e., unmeasurable (log)viral load, were compared between the treatment groups using a logistic regression model with fixed effects for randomized group, age, and baseline comorbidity (yes/no). Separate models were fitted for Days 2, 4, and 6. Imputation as in the analysis of the endpoint were followed for this analysis. Rates and 95% CIs were calculated using least-square means.
3. Time-to SARS-CoV-2 (log₁₀) Viral Load Reduction Below a Range of Thresholds (1.00, 2.00 and 3.00) at Day 2, Day 4 and Day 6—Time to Ct threshold (unmeasurable viral load) was modeled using a Cox proportional hazards model including fixed effects for treatment group, age, and baseline co-morbidity. Individuals who did not achieve threshold were considered censored at the last available Ct measurement time. Median time and 95% confidence intervals were calculated.
4. Proportion of subjects requiring hospitalization or ED/ER visits by day 18—Subjects will be modeled using a logistic regression with fixed effects as in the analysis (arm, age, co-morbidity) for this assessment. Point estimates and 95% confidence intervals will be provided. Subjects for whom hospitalization or ER/ED visits cannot be definitively determined for day 18 will be imputed as having a visit. This may include some blinded data review by the sponsor for subjects who are deemed recovered, and withdrew consent or were lost to follow-up prior to day 18.
5. Proportion of Subjects Experiencing Modified Jackson Scores Change from Baseline ≥5 or Reduction to Zero at Days 2, 4, 6, 9 and 18—Modified Jackson scores were modeled as in the analysis of the endpoint, but all days from randomization through Day 6 were used each with an indicator for whether on or prior to treatment. Symptom scores on the day treatment was initiated were considered prior to treatment unless times of NONS administration and questionnaire completion indicated the opposite. Scores that were missing due to hospitalization, or death, were imputed with the maximum possible score. If scores were missing and a subject subsequently died or was hospitalized prior to Day 18, they were also imputed with the maximum possible score. All other missingness were treated as missing at random and all available data for the subject included in the model.
6. Proportion of Subjects Experiencing Modified Jackson Scores Change from Baseline ≥5 or Reduction to Zero at Days 2, 4, 6, 9 and 18—Proportion of subjects experiencing modified Jackson score (or COVID-19 PRO Score) change of ≥5 from baseline or reduction to zero were modeled using a logistic regression as described for other secondary endpoints at Day 2, 4, 6, 9, and 18. Scores that were missing due to hospitalization, or death, were imputed with the maximum possible score. Scores that were missing and a subject subsequently died or was hospitalized were also imputed with the maximum possible score. All other missingness were treated as missing at random.

The proportion of subjects who discontinued NONS or control treatment prior on-treatment (Days 1 to 9) were estimated and compared using a Fisher's Exact test. Severity and frequency of adverse events as well as clinically significant changes in oximetry and symptoms (if available) were summarized and presented by arm and period (on-treatment [Days 1 to 9], post-treatment [Days 10 to 18]) in tabular form.

### EXAMPLE 3-H—Sample Size and Population

The sample size for the study was based on the endpoint, however there was insufficient information available regarding the correlation of the Ct results over time within-subjects, precluding a sample size calculation involving unfounded assumption. Instead, the sample size was justified using a single-timepoint and assumed to be a lower bound on power considering the inclusion of additional time-points in the analysis.

A sample size of 50 subjects (25 per arm) was determined to have 91% power to demonstrate superiority of treatment versus control if the true underlying mean Cts were 31 versus 26, respectively, using a two-sided 0.05-level Wilcoxon-signed rank test and assuming a common standard deviation of 5 for both groups.

These assumptions were based on unpublished CDC data available online where specimens from which replication-competent virus was recovered had an average Ct of 26 (N1, N2, N3 targets) compared to 35 (N2 and N3 targets) when not recovered with an estimated standard deviations of 3.8 and 5 for these groups, respectively.

The lower mean Ct for the treatment group assumed some attenuation due to variable treatment effect and therefore a lower average Ct. The assumed standard deviation of 5 was conservative and should have sufficiently accommodated the variability of treatment effect since those with live virus present tended to have lower variance.

Safety Population: The safety population included all subjects who received at least 1 dose of study treatment. Participants were analyzed, according to the intervention, they actually received. Safety results are to be separated into events on-treatment (Day 1-9) and off-treatment (Day 10-18).

Intent-to-treat (ITT) Population: The ITT population included all subjects who were enrolled and randomized irrespective of adherence to study protocol or study treatments received. All subject data were analyzed according to their assigned randomization group.

Per-Protocol (PP) Population: The PP population consisted of all subjects who were enrolled in the study, had been randomized, received at least one dose of their assigned study treatment and had no major protocol deviations, were not lost-to-follow-up for reasons unrelated to treatment, and had documented test article administration on a minimum of 80% of their on-study days while enrolled in the study. All subject data was analyzed according to their received treatment.

Demographics and other baseline characteristics were summarized for the safety population by treatment group and overall, with descriptive statistics including n, mean, and SD for numeric variables and frequency and percentage for categorical variables. Demographics included age, gender, and race. Medical history included comorbidities (any, or chronic heart, liver, and lung disease; diabetes; and hypertension) and presenting symptoms (dry cough, fever, loss or sense of smell, or none). Disposition was summarized by efficacy for the NONS treatment, placebo and overall. The number and percentage of subjects discontinuing, along with the reasons for discontinuation, were summarized for subjects in the safety population. The analysis of the efficacy endpoint was carried out on the observed data, i.e., a complete case analysis. All analyses of demographics, secondary endpoints, and safety data were based on the observed data.

### EXAMPLE 3-I—Study Subjects

A total of 183 potential participants were screened for the trial. Subjects had mild COVID-19 infection defined by non-pneumonia or mild pneumonia with no chest pain or shortness of breath, where pneumonia was defined as ‘inflammation of one or both lungs’. Mild symptoms included, but were not limited to fever (>37.2° C.), dry cough, tiredness, sore throat, malaise, headache, muscle pain, lack of taste or smell and gastrointestinal symptoms.

One hundred subjects did not meet eligibility criteria, and 83 subjects were enrolled and randomized into the trial. One patient withdrew consent prior to receiving any randomized treatment. Two randomized subjects were not able to initiate treatments because their treatment packs were damaged and were excluded from further participation.

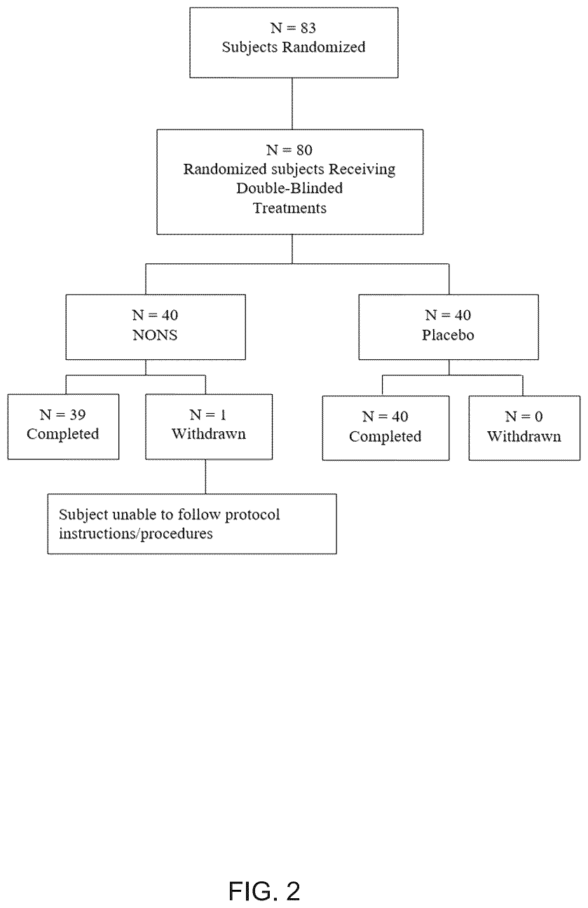

Overall, a total of 80 subjects (40 NONS, 40 Placebo) were randomized into the trial and received study treatments as depicted in FIG. 2. One subject in the NO nasal spray treatment group was prematurely discontinued unrelated to adverse events by the investigator for an inability to follow protocol instructions and procedures. The subject's data was included in the ITT population efficacy analyses datasets, but excluded from the PP population efficacy analyses datasets.

The number of subjects randomized, completing the trial and withdrawn per treatment group assignment are presented in Table 3I-1. No subject withdrew due to an adverse event (AE), death, was lost-to-follow-up, or due to the Sponsor's closure of the trial. Over the course of the study, 41.7% of the NONS doses and 46.3% of placebo doses were recorded as having been administered; 4.3% of the NONS doses and 4.8% of placebo doses were recorded as having been missed.

TABLE 3I-1
Subject Disposition by Treatment and Overall

| | NONS | Placebo | Overall |
|---|---|---|---|
| | (N = 40) | (N = 40) | (N = 80) |
| Enrolled | 40 | 40 | 80 |
| Completed | 39 | 40 | 79 |
| | (97.5%) | (100%) | (98.75%) |
| Withdrawn | 1 | 0 | 1 |
| | (2.5%) | | (1.25%) |
| Reason for Withdrawal | | | |
| Adverse Event | 0 | 0 | 0 |
| Subject Decision | 0 | 0 | 0 |
| Physician Decision | 1 | 0 | 1 |
| Death | 0 | 0 | 0 |
| Lost to Follow Up | 0 | 0 | 0 |
| Sponsor Closed Study | 0 | 0 | 0 |
| Other Reason | 0 | | 0 |
| | (2.5%) | | (1.25%) |

No protocol deviations were recorded as having occurred during the execution of the study. However, 54.0% of the NONS doses and 49.0% of placebo doses did not have data recorded as being administered or missed. Overall, no irregularities occurred during the study. No issues of significant noncompliance occurred which would have impacted the results of the trial. No geographical area on the trial reported any irregularity with regard to the drug product.

### EXAMPLE 3-J—Efficacy Evaluation

The ITT population consisted of all subjects that had been enrolled and randomized irrespective of adherence to study protocol or study treatments received. This population was used for the efficacy analyses. No subject was excluded from the ITT population data.

The per protocol (PP) population includes all study subjects that had been enrolled, randomized, and received a minimum of 80% of on-study treatments. In addition, no subject had been lost-to-follow-up, withdrew consent, and had no major protocol deviations while enrolled in the study. The safety population consisted of all subjects who were enrolled in the clinical trial and had received at least 1 dose of study treatment. Safety results are separated into events occurring on-treatment (Day 1-9) and off-treatment (Day 10-18).

Demographics for the safety population are presented in Table 3J-1. Overall, 36.3% of subjects were male and 63.7% of subjects were female. The mean subject age was 44.0 years, majority of subjects were White (85.0%), and 23.8% of subjects were extremely obese (BMI≥30).

Demographic characteristics were generally similar between the treatment groups, with the exception that more subjects were White in the placebo group and extremely obese in the NONS group (p=0.034; although 22.5% NONS and 7.5% placebo had missing data).

TABLE 3J-1
Demographics and Baseline Characteristics (Safety Population)

| | NONS | | Placebo | | All Subjects | |
|---|---|---|---|---|---|---|
| | n (%)^{a} | | n (%)^{a} | | n (%)^{a} | |
| | (N = 40) | | (N = 40) | | (N = 80) | |
| Age (years) | | | | | | |
| Mean (SD) | 44.1 | (12.1) | 43.9 | (12.6) | 44.0 | (12.4) |
| Gender (%) | | | | | | |
| Male | 16 | (40.0) | 13 | (32.5) | 29 | (36.3) |
| Female | 24 | (60.0) | 27 | (67.5) | 51 | (63.7) |
| Race (%) | | | | | | |
| Asian | 2 | (5.0) | 3 | (7.3) | 5 | (6.2) |
| Black African | 1 | (2.5) | 0 | | 1 | (1.25) |
| (Caribbean) | | | | | | |
| Mixed | 2 | (5.0) | 0 | | 2 | (2.5) |
| White | 34 | (85.0) | 37 | (92.5) | 71 | (88.8) |
| Other | 1 | (2.5) | 0 | | 1 | (1.25) |
| Obesity (%) | | | | | | |
| BMI ≥ 30 | 12 | (30.0) | 7 | (17.5) | 19 | (23.8) |
| BMI < 30 | 19 | (47.5) | 30 | (75.0) | 49 | (61.2) |
| No Data | 9 | (22.5) | 3 | (7.5) | 12 | (15.0) |

^{a}n = number of subjects with data available.

Other baseline characteristics for the safety population are presented in Table 3J-2. Over-all, 12.5% of subjects exhibited a comorbidity, with chronic lung disease being reported in the NONS group (5.0%); 6.25% of subjects were hypertensive and 6.25% were diabetic.

Overall, 61.3% of subjects presented with a dry cough, 28.8% with a fever, 17.5% had a loss of the smell, while 16.3% of subjects were asymptomatic. COVID-19 presenting symptoms were generally similar between the treatment groups, with the exception that more subjects had a fever in the placebo group (p=0.081). The number of asymptomatic subjects in the placebo group (15.0%) were comparable to the NONS group (17.5%; p=0.743).

TABLE 3J-2
Other Baseline Characteristics (Safety Population)

| | NONS | | Placebo | | All Subjects | |
|---|---|---|---|---|---|---|
| | n (%)^{a} | | n (%)^{a} | | n (%)^{a} | |
| | (N = 40) | | (N = 40) | | (N = 80) | |
| Comorbidities | | | | | | |
| Any comorbidity | 6 | (15.0) | 4 | (10.0) | 10 | (12.5) |
| Chronic lung disease | 2 | (5.0) | 0 | | 2 | (2.5) |
| Chronic heart disease | 0 | | 0 | | 0 | |
| Chronic liver disease | 0 | | 0 | | 0 | |
| Cardiovascular | | | | | | |
| Hypertension | 2 | (5.0) | 3 | (7.5) | 5 | (6.25) |
| Metabolic Disorders | | | | | | |
| Diabetes | 3 | (7.5) | 2 | (5.0) | 5 | (6.25) |
| Presenting COVID-19 | | | | | | |
| Symptoms | | | | | | |
| Dry Cough | 22 | (55.0) | 27 | (67.5) | 49 | (61.3) |
| Fever | 7 | (17.5) | 16 | (40.0) | 23 | (28.8) |
| Loss of Sense of Smell | 9 | (22.5) | 5 | (12.5) | 14 | (17.5) |
| Asymptomatic | 6 | (15.0) | 7 | (17.5) | 13 | (16.3) |
| No Data | 5 | (12.5) | 3 | (7.5) | 8 | (10.0) |

^{a}n = number of subjects with data available.

Treatment compliance was recorded by subjects via the daily treatment questionnaire, i.e., nasal sprays administered or missed per day. Returned completed, partially used, and unused treatments for each subject were summarized by the trial center. Verification of subjects' treatment compliance from the end of study follow-up call were summarized by the trial center.

The objective was to demonstrate the efficacy of NONS to shorten COVID-19 infectivity duration in mild COVID-19 patients. Fifty-four (54) nasal spray treatments were administered 6 times per day for 9 continuous days. Each treatment considered of 4 sprays (560 μL total), 2 sprays per nostril, for a total dosage regimen of approximately 30 mL. The efficacy variable was the change from baseline reduction through Day 6 of treatment in ‘Cycle Threshold’, i.e., the difference in SARS-CoV-2 viral load.

The efficacy endpoint for this trial is the comparison of the mean (log₁₀) viral load reduction change from baseline between treatment groups. The analysis performed utilized a linear mixed effect model for (log)viral load with fixed effects for randomized treatment group (NONS, control), age (continuous), presence of comorbidities at randomization (no/yes), study day (0, 2, 4, 6), and interaction between treatment group and each study day. Subjects missing all four (log)viral load measurements (baseline, Day, 2, 4 and 6) were imputed with the highest (log)viral load observed in either group for the analysis. A random (Gaussian) intercept for subject was included to account for multiple observations (full model).

Parameter estimation was performed using maximum likelihood (not restricted maximum likelihood/REML) and inference based on the likelihood ratio test of the full model versus a null model, excluding the main and interaction effects involving the groups. In addition to this overall test for treatment effect, least-square mean (log)viral load estimates for each timepoint were generated from the full model accompanied by 95% confidence.

Parameter estimation was performed using maximum likelihood (not restricted/REML). The efficacy results for the intent-to-treat (ITT) population are presented in Table 3J-3.

The mean baseline SARS-CoV-2 viral load was 2.415×10¹³ RNA copies per cm³ (mL) across all study subjects. The SARS-CoV-2 viral load was significantly decreased over the first 6 days of treatment. In addition, the viral load observed in subjects randomized to placebo was significantly higher over the first 6 days compared with subjects allocated to the NONS active treatment group. The treatment difference between NONS and placebo was statistically significant for all three days (p<0.05 at Day 2, 4 and 6 for NONS treatments compared to placebo).

TABLE 3J-3
Summary of Treatment Difference for SARS-CoV-2 (log)Viral Load
Change from Baseline at Day 2, Day 4 and Day 6 (Generalized Linear
Model with Fixed Effect for Treatment Group; ITT Population)

| Predictors^{a} | Estimates^{b} | 95% CI^{c} | P-Value | Estimates | 95% CI | P-Value |
|---|---|---|---|---|---|---|
| Day 2 | −1.33 | −1.92 to −0.73 | <0.001 | −0.74 | −1.17 to −0.31 | 0.001 |
| Day 4 | −2.34 | −2.93 to −1.75 | <0.001 | −1.76 | −2.19 to −1.34 | <0.001 |
| Day 6 | −3.24 | −3.84 to −2.65 | <0.001 | −2.79 | −3.22 to −2.37 | <0.001 |
| Treatment Difference | 1.17 | 0.33 to 2.01 | 0.006 | | | |
| Day 2 | | | | | | |
| Treatment Difference | 1.15 | 0.31 to 1.99 | 0.007 | | | |
| Day 4 | | | | | | |
| Treatment Difference | 0.90 | 0.06 to 1.74 | 0.035 | | | |
| Day 6 | | | | | | |

^{a}The analysis was performed utilizing a generalized linear mixed effect model for (log₁₀) viral load with fixed effects for randomized treatment group (NONS, control), age (continuous), presence of comorbidities at randomization (no/yes), study day (0, 2, 4, 6), interaction between treatment group and study day. Subjects missing all four Viral Load measurements (baseline, Day, 2, 4, and 6) were imputed with the highest (log)viral load observed in either group. A random (Gaussian) intercept for subject was included to account for multiple observations.
^{b}Parameter estimation were performed using maximum likelihood (not restricted/REML) and inference was based on the full model versus a null model excluding the main and interaction effects involving arm.

In order to assess the impact of the main covariate (treatment group) on viral load over time, the null model was performed excluding the main covariate and the interaction effect between the main covariate and the study day. The full model and the null model were compared using a likelihood ratio test.

The likelihood ratio test suggests that the full model significantly differs from the null model. The combination of the assessment day and use of active treatment significantly predict the viral load level. The null hypothesis is therefore excluded.

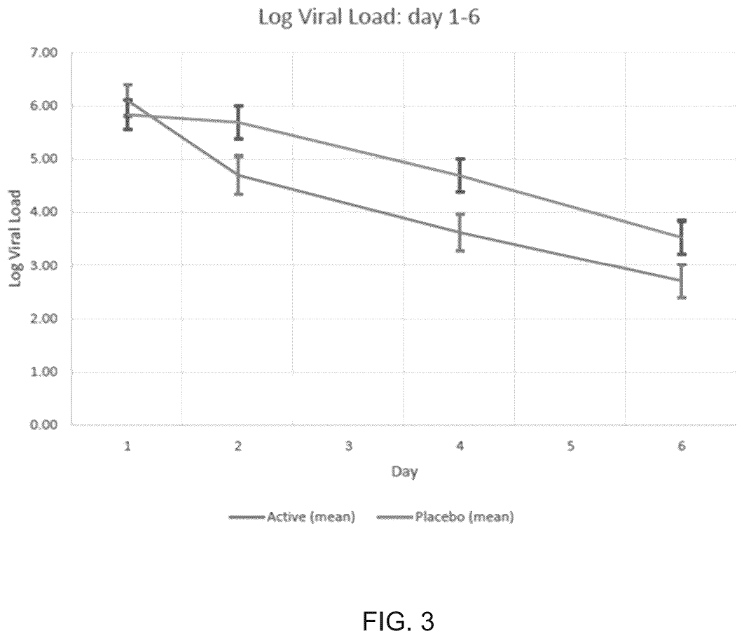

A graphical representation of the (log)viral load change from baseline to Days 2, 4 and 6 is shown in FIG. 3. The mean treatment difference using the area under curve from Baseline through Day 6 was −5.220 with a 95% CI −9.136 to −1.305 (p=0.01). A rapid reduction (95%) in the high SARS-CoV-2 viral load was observed within 24 hours, and a 99% reduction within 72 hours, with NONS treatments.

As a scenario analysis, a second model will be constructed using random effects for treatment group. A further scenario will repeat the analysis using the per protocol population, excluding any patients with missing data.

The analysis was repeated utilizing a linear mixed effect model for (log₁₀) viral load with random effects for randomized treatment group (NONS, control), age (continuous), presence of comorbidities at randomization, study day (0, 2, 4, 6), and interaction between treatment group and each study day. A random (Gaussian) intercept for subject was included to account for multiple observations (full model). Parameter estimation was performed using maximum likelihood (not restricted maximum likelihood/REML) and inference based on the likelihood ratio test of the full model versus a null model, excluding the main and interaction effects involving the groups. In addition to this overall test for treatment effect, least-square mean (log)viral load estimates for each timepoint were generated from the full model accompanied by 95% confidence.

Parameter estimation was performed using maximum likelihood (not restricted/REML). The efficacy results for the intent-to-treat (ITT) population are comparable to the efficacy results (fixed effect). The likelihood ratio test also suggests that the full model significantly differs from the null model. The secondary efficacy endpoint analyses broadly support the efficacy of nitric oxide nasal spray administration in mild COVID-19 patients.

Mean difference in the log viral load over the first 6 continuous days of treatment was compared between treatment groups using a repeated measures t-test for the ITT population. Specific separate analyses were performed for the change in log viral load at Day 2, Day 4 and Day 6 compared to Baseline for the NONS and Placebo groups, as depicted in Table 3J-4.

The change in baseline reduction in SARS-CoV-2 (log₁₀) viral load were statistically significant at Day 2 (p=0.008) and Day 4 (p=0.021) on NONS treatments compared to Placebo. The CFB reduction in SARS-CoV-2 (log)viral load was greater, but not statistically significant at Day 6 (p=0.094) on NONS treatments compared to Placebo. Overall, these results are consistent with the result outcomes.

TABLE 3J-4
Mean SARS-CoV-2 (log)Viral Load Change from Baseline
at Day 2, Day 4 and Day 6 (ITT Population)

| | NONS^{a} | Placebo^{a} | | | |
|---|---|---|---|---|---|
| | Mean Change | Mean Change | Mean | | |
| | from Baseline | from Baseline | Treatment | | |
| | (N = 40) | (N = 40) | Difference | 95% CI^{b} | P-Value^{c} |
| Day 2 | −1.328 | −0.154 | −1.174 | −2.024 | 0.008 |
| | | | | to −0.323 | |
| Day 4 | −2.340 | −1.186 | −1.153 | −2.126 | 0.021 |
| | | | | to −0.181 | |
| Day 6 | −3.344 | −2.341 | −0.903 | −1.961 | 0.094 |
| | | | | to 0.156 | |

^{a}Change in baseline reduction in SARS-CoV-2 (log₁₀) viral load measured as cycle threshold (Ct). Ct is reported as a whole number to quantitate changes in viral load from nasal swabs without the need for viral cultures. Ct correlates inversely to live virus and viral load and was used as the endpoint measurement to determine the antiviral effectiveness of the treatments.
^{b}95% CI = 95% confidence interval.
^{c}Two-tailed probability

All viral samples were sequenced for the presence of known SARS-CoV-2 variants of concern (VOC). Thirty-four (87.2%) of the NONS group subjects were determined to have the B.1.1.7 lineage (VOC202012/01) with the remainder determined not to be VOC. Thirty-four (85.0%) of the placebo group subjects were determined to be B.1.1.7 lineage with the remainder determined not to be a known variant of concern.

The proportion of subjects achieving a range of SARS-CoV-2 (log₁₀) viral load thresholds, i.e., (log)viral load 1, 2, and 3 (Ct threshold with unmeasurable viral load) at Day 2, Day 4 and Day 6 were compared between the treatment groups using a logistic regression model with fixed effects for randomized group, age, and baseline comorbidity. Separate models were fitted for Days 2, 4, and 6, as depicted in Table 3J-5. Imputation similar to that performed in the analysis of the endpoint were followed for this analysis. Rates and 95% CIs were calculated using least-square means.

No statistically significant difference between NONS treatment and placebo at any of the three threshold values were established, with the exception of the day 4 analysis for the (log) viral load threshold of 3 (p=0.012). A numerical benefit for NONS treatment was observed in all analyses, with the exception of Day 2 analysis for the log viral load threshold of 1. The study was likely underpowered to demonstrate a consistent statistically significant impact for this secondary endpoint. No additional analyses were conducted on the PP population.

TABLE 3J-5
Proportion of Subjects Achieving SARS-CoV-2 (log)Viral
Load Reduction Below a Range of Thresholds at
Day 2, Day 4 and Day 6 (ITT Population)

| | NONS | | |
|---|---|---|---|
| | Compared | | |
| | to Placebo | | |
| | Odds Ratio | 95% CI^{b} | P-Value |
| (log)Viral Load | | | |
| Threshold ≤ 1.00^{a} | | | |
| Day 2 | 0.186 | 0.019 to 1.844 | 0.151 |
| Day 4 | 1.991 | 0.434 to 9.124 | 0.375 |
| Day 6 | 3.033 | 0.831 to 11.069 | 0.093 |
| (log)Viral Load | | | |
| Threshold ≤ 2.00^{a} | | | |
| Day 2 | 5.377 | 0.542 to 53.311 | 0.151 |
| Day 4 | 1.991 | 0.434 to 9.124 | 0.375 |
| Day 6 | 3.033 | 0.831 to 11.069 | 0.093 |
| (log)Viral Load | | | |
| Threshold ≤ 3.00^{a} | | | |
| Day 2 | 2.015 | 0.582 to 6.974 | 0.269 |
| Day 4 | 4.179 | 1.372 to 12.724 | 0.012 |
| Day 6 | 1.208 | 0.491 to 2.973 | 0.681 |

^{a}Change in baseline reduction in SARS-CoV-2 (log₁₀) viral load measured as cycle threshold (Ct). Ct is reported as a whole number to quantitate changes in viral load from nasal swabs without the need for viral cultures. Ct correlates inversely to live virus and viral load and was used as the endpoint measurement to determine the antiviral effectiveness of the treatments.
^{b}95% CI = 95% confidence interval.

The difference in time to reduction of SARS-CoV-2 (log₁₀) viral load below a range of thresholds (1, 2 and 3), over treatment Days 2, Day 4 and Day 6 from baseline, were modeled to provide an alternative method of assessing if NONS treatments reduced nasal viral load faster than Placebo.

The models for each of the three threshold evaluations used a Cox proportional hazards ratio including fixed effects for treatment group, age, and baseline co-morbidity. Individuals who did not achieve threshold were considered censored at the last available Viral load measurement time. The median time and 95% confidence intervals were calculated for each threshold, and results over the 6 treatment days were displayed via Kaplan Meier curves.

The hazard ratio results for each of three (log)viral load thresholds for the treatment group comparisons are summarized in Table 3J-6. None of the differences between the NONS treatments and placebo at each of the thresholds were statistically significant. No additional analyses were conducted on the PP population.

TABLE 3J-6
Summary of the Treatment Difference in Time to SARS-CoV-2
(log)Viral Load Reduction Below a Range of Thresholds
(1, 2 and 3) at Day 2, Day 4 and Day 6 (ITT Population)

| Hazard Ratio | Survival | 95% | |
|---|---|---|---|
| Result NONS | Time | Confidence | |
| Compared to Placebo | Estimate^{a} | Interval | P-Value |
| (Log)Viral Load | 0.48 | 0.16 to 1.44 | 0.191 |
| Threshold of 1.00 | | | |
| (Log)Viral Load | 0.61 | 0.30 to 1.25 | 0.176 |
| Threshold of 2.00 | | | |
| (Log)Viral Load | 0.73 | 0.41 to 1.30 | 0.284 |
| Threshold of 3.00 | | | |

^{a}Values for the effects of age and comorbidities for each threshold (not displayed) were not statistically significant.

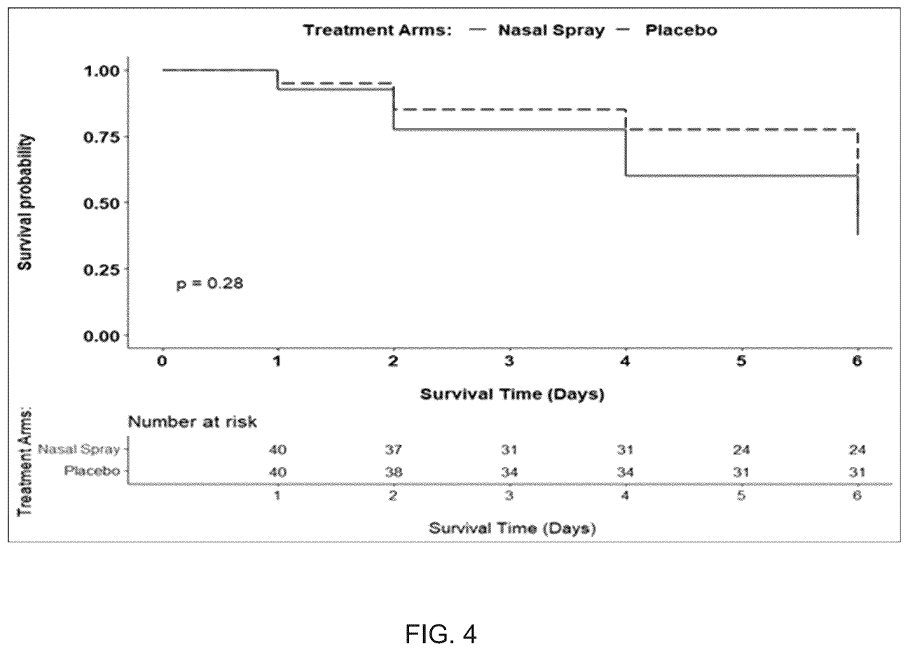

The survival probabilities for the time to SARS-CoV-2 (log₁₀) viral load reduction at Day 2, Day 4 and Day 6 are displayed as Kaplan Meier curves for each of the thresholds as shown in FIG. 4. The median and mean times (95% CI) for all or most of the thresholds could not be reported since few treatments reached 0.5. The Kaplan Meier curve for the time to (log)viral load threshold 3 achievement is displayed in FIG. 4. The threshold 3 median time was the same for both the NONS treatment and Placebo (6 hours), while the mean time was 4 hours on NONS treatment and 6 hours for Placebo. No additional analyses were conducted on the PP population.

Subjects were to be modeled using a logistic regression with fixed effects as in the analysis (arm, age, co-morbidity) for this assessment. Point estimates and 95% confidence intervals were to be provided. Subjects for whom hospitalization or ER/ED visits could not be definitively determined for Day 18 were to be imputed as having a visit. This could have included some blinded data reviewed by the sponsor for subjects who were deemed recovered, and withdrew consent or were lost to follow-up prior to Day 18.

Two subjects had hospitalization (one in each treatment group) which were considered non-treatment related and not adverse events per the investigator. No additional ED/ER visits were used by patients subsequent to their recovery. No statistical analyses were carried out for this outcome on the ITT or PP populations.

Modified Jackson scores, i.e., COVID-19 Patient Reported Outcome (PRO) Scores were derived from data collected on twelve symptom questions asked each day (Day 1-9) regarding: nasal congestion/runny nose, new loss of smell/taste, muscle or body aches, sore/scratchy throat, cough, sneezing, headache, malaise, fever/chills, shortness of breath, nausea/vomiting, and diarrhea. For each, the subject was to provide an answer of absent (0), mild (1), moderate (2) or severe (3). Total symptom scores could range from 0 to 36.

Scores were modeled using a GLM, as in the analysis of the endpoint, for all days from randomization through Day 9. Scores that were missing due to hospitalization or death were imputed with the maximum possible score. If scores were missing and a subject subsequently died or was hospitalized prior to Day 9, they were imputed with the maximum possible score. All other missingness were treated as missing at random and all available data for the subject included the model.

Results were available for 48 of the 79 subjects in the PP population on Day 1, declining to 32 of the 79 subjects on Day 9. A descriptive statistically analysis for the aggregate total Modified Jackson symptom scores on each of the 9 days is listed in Table 3J-7. There was no statistically significant difference between treatments for any of the 9-day aggregate total scores.

TABLE 3J-7
Daily Modified Jackson Total Symptom Scores (Day 1-9) (PP)

| | | | | | | | | |
|---|---|---|---|---|---|---|---|---|
| Day 1 | Day 2 | Day 3 | Day 4 | Day 5 | Day 6 | Day 7 | Day 8 | Day 9 |

NONS

| | | | | | | | | | |
|---|---|---|---|---|---|---|---|---|---|
| N | 23 | 22 | 21 | 19 | 19 | 17 | 16 | 16 | 16 |
| Mean | 7.35 | 7.18 | 6.67 | 5.74 | 5.42 | 4.47 | 4.38 | 4.00 | 4.25 |
| Median | 7.00 | 5.00 | 5.00 | 3.00 | 3.00 | 3.00 | 3.00 | 2.50 | 2.50 |

Placebo

| | | | | | | | | | |
|---|---|---|---|---|---|---|---|---|---|
| N | 25 | 25 | 25 | 23 | 22 | 19 | 19 | 18 | 16 |
| Mean | 8.52 | 7.24 | 6.28 | 5.30 | 4.77 | 4.00 | 3.95 | 2.56 | 1.75 |
| Median | 9.00 | 6.00 | 5.00 | 4.00 | 4.00 | 3.00 | 3.00 | 1.00 | 1.00 |
| p (NONS vs | 0.255 | 0.341 | 0.799 | 0.603 | 0.519 | 0.482 | 0.442 | 0.972 | 0.347 |
| Placebo | | | | | | | | | |
| Treatment | | | | | | | | | |
| Difference)^{a} | | | | | | | | | |

^{a}Mann-Whitney test

A repeat serial measurements analysis was carried out on the 32 subjects with symptom scores documented throughout the 9-day period. The scores from baseline to day 9 were a mean reduction in the NONS group of −5.50 (±4.033) and for the placebo group of −4.38 (±5.110), for a treatment difference of 1.125 (p=0.495).

An AUC analysis was also carried out on the serial measurements assessment from baseline to day 9 to evaluate the impact of all intermediate data points. The baseline values for each curve was set to zero, in order to take into account differences in starting symptom scores. The AUC from baseline to day 9 was a mean of 46.19 (±50.349) for the NONS group and for the placebo group of 32.56 (±18.975), for a treatment difference of 13.63 (p=0.319).

The proportion of subjects experiencing modified Jackson scores, i.e., COVID-19 PRO Scores change from baseline of ≥5 or a reduction to zero were to be modeled using a logistic regression as described for other secondary endpoints at Day 2, 4, 6, and 9. Scores that were missing due to hospitalization or death were to be imputed with the maximum possible score. Scores that were missing and a subject subsequently died or was hospitalized were also to be imputed with the maximum possible score. All other missingness were to be treated as missing at random. No analysis was undertaken for the modified Jackson symptom scores change from baseline of greater than or equal to 5 for either the ITT or PP populations.

The time-to-event analysis to achieve a sustained symptom score of zero was limited in that there were 11 subjects overall that achieved this endpoint. Due to this small sample, a second analysis based on the achievement of a sustained symptom score of <3 was performed which yielded 32 subjects.

In both cases, the Kaplan-Meier curves showed an apparent benefit for NONS, although not statistically significant in either case (p=0.498 and p=0.653, respectively). However, subjects treated with placebo had a higher baseline symptom score, and consequently, if there is no difference between treatments, the time to reach a fixed threshold will inevitably be greater with placebo.

During the study an interim analysis was supplied in strict confidence to the independent data monitoring committee (DMC). The DMC requested such analysis at a frequency relevant to the emerging data from the current trial and other NO studies.

The statistical results on the viral load data from the first 52 completed subjects suggested an improved virus load clearance time for subjects treated with NONS compared to placebo (AUC for Days 1 to 6=−10.803 vs −6.702; p=0.088). There was no adverse safety signal observed.

The DMC extend the study, since the original power calculation used a number of assumptions including a clear lack of understanding of the clinical behavior of early-stage COVID-19 at the time the study was designed. A recalculation of the sample size, based on the actual observed trial performance, estimated a total of 45-50 subjects per arm (90 to 100 total). Recruitment could be extended to a total of 55 subjects per arm (110 total) as a buffer against attrition.

One geographical site in England was used to randomized subjects (St. Peter's Hospital/Clinics, Guildford St. Lyne, Chertsey). Subjects self-administered the nasal spray solution. Pooling of sites to create one larger virtual sites did not occur.

The protocol describes the endpoint population and analysis. A slight adjustment was made for the endpoint. The analyses as documented in the protocol as originally plan was predicated on having semi-quantitative metrics for viral load (Ct). The current analyses uses a fully quantitative estimate of the viral load.

The secondary endpoints are stated in the protocol. No adjustments to the Type I error for secondary efficacy endpoints were made to account for the multiple endpoints, as all tested hypotheses were considered exploratory.

The evaluable efficacy population consisted of the full analysis ITT population. The PP population consisted of the group of subjects that were not lost-to-follow-up, had not withdrawn consent, and had no major protocol violations that could influence efficacy. Results were generally consistent for the two populations.

The objective was to demonstrate the efficacy of NONS to shorten COVID-19 infectivity duration in mild COVID-19 patients (ITT Population). The efficacy variable was the change from baseline reduction through Day 6 of treatment in ‘Cycle Threshold’, i.e., the difference in SARS-CoV-2 viral load. The analysis performed utilized a linear mixed effect model for (log)viral load with fixed effects for the randomized treatment groups. The mean baseline SARS-CoV-2 viral load was 2.415×10¹³ RNA copies per cm³ (mL) across all study subjects.

SARS-CoV-2 (log₁₀) Viral Load Reduction: Viral load was significantly decreased over the first 6 days of treatment with the NO Nasal Spray Treatments compared to Placebo. The treatment difference was statistically significant for Day 2 (p=0.006), Day 4 (p=0.007), and Day 6 (p=0.035), respectively. The PP population results were comparable to the ITT population results.

Likelihood Ratio Test: Impact of the main covariate (treatment group) on viral load over time was assessed using the full model compared to a null model, excluding the main covariate and the interaction effect between the main covariate and the study day. The likelihood ratio test suggests that the full model significantly differs from the null model (p=0.01). The combination of the assessment day and use of active treatment significantly predict the viral load level. The null hypothesis is therefore excluded. The PP population results were comparable to the ITT population results.

Area Under the Curve (log₁₀) Viral Load Change from Baseline: Mean treatment difference using the area under curve from baseline (CFB) over the first 6 days of NONS treatment was −5.220 with a 95% CI −9.136 to −1.305 (p=0.01). Arapid reduction (95%) in the high SARS-CoV-2 viral load was observed within 24 hours, and a 99% reduction within 72 hours, with NONS treatments. The PP Population results were comparable to the ITT Population results.

SARS-CoV-2 (log₁₀) viral load reduction and the likelihood ratio test using sensitivity analyses with random effects for treatment groups was comparable to the fixed effects assessment.

Secondary efficacy analyses for the most part broadly support the efficacy of nitric oxide nasal spray treatment in mild COVID-19 patients.

Mean SARS-CoV-2 (log₁₀) Viral Load CFB at Day 2, Day 4 and Day 6: The change in baseline reduction in SARS-CoV-2 (log) viral load were statistically significant at Day 2 (p=0.008) and Day 4 (p=0.021) with NONS treatments compared to Placebo. The CFB reduction in SARS-CoV-2 (log)viral load was greater, but not statistically significant at Day 6 (p=0.094) on NONS for the ITT population. Similar results were observed for the PP population.

Proportion of Subjects Achieving SARS-CoV-2 (log₁₀) Viral Load Reduction Below Threshold Ranges: No statistically significant difference between NONS treatment and placebo at any of the three threshold values were established, with the exception of the day 4 analysis for the (log) viral load threshold of 3 (p=0.012) for the ITT population. Similar results were observed for the PP population.

Time to SARS-CoV-2 (log₁₀) Viral Load Reduction Below a Range of Thresholds: None of the differences between the NONS treatments and placebo at each of the thresholds were statistically significant for the ITT population. No analyses were conducted on the PP population.

Modified Jackson Scores Modeled on the Endpoint Analysis: There was no statistically significant difference between treatments for any of the 9-day aggregate total scores.

Proportion of Subjects Experiencing Modified Jackson Scores Change from Baseline ≥5 or Reduction to Zero at Days 2, 4, 6 and 9: No statistical analyses were carried on the proportion of subjects experiencing a CFB 5 or reduction to zero at Days 2, 4, 6, 9 and 18. A time-to-event analysis to achieve a sustained symptom score of zero to Day 9 was performed which showed an apparent benefit for NONS, although not statistically significant. No statistical analyses were carried out on the ITT and PP populations for the proportion of subjects requiring hospitalization or ED/ER visits.

Overall, a difference in treatment across the efficacy and multiple secondary efficacy endpoints was observed between NONS and placebo. SARS-CoV-2 viral loads were immediately and consistently lower with NONS treatments, suggesting a shortened COVID-19 infectivity duration in mild COVID-19 patients. All viral samples were sequenced for the presence of known SARS-CoV-2 variants of concern (VOC). Thirty-four (87.2%) of the NONS group subjects were determined to have the B.1.1.7 lineage (VOC202012/01) with the remainder determined not to be VOC. Thirty-four (85.0%) of the placebo group subjects were determined to be B.1.1.7 lineage with the remainder determined not to be a known variant of concern. A PHE matched cohort analysis has reported a death risk ratio for VOC-infected individuals compared to non-VOC of 1.65 (95% CI 1.21-2.25). It is therefore a possibility VOC B.1.1.7 infection is associated with an increased risk of mortality compared to infection with non-VOC viruses.

### EXAMPLE 3-K—Safety Evaluation

NONS treatments were well-tolerated and considered safe by the investigator, in that:
No deaths and no life-threatening TEAEs occurred during the study. No subject withdrew from the study due to an AE.
No subjects reported experiencing a TEAE per the investigator related to treatment. One subject in each treatment group reported a hospitalization unrelated to treatment.
There were no complications, misuse, abuse or overdose situations with the self-administration of NONS.

Overall, the 54 total nitric oxide nasal spray treatments, administered 6 times per day for 9 continuous days, with each treatment of 4 sprays (560 μL total), delivered as 2 sprays per nostril for a total dosage regimen of approximately 30 mL was well-tolerated in this Phase IIb efficacy and safety study. No new safety concerns were identified with this novel NO therapy (NONS) and its administration to mild COVID-19 patients.

### EXAMPLE 3-L—Conclusions

The nasal cavity is the major route for host entry and infectivity of the COVID-19 virus. An effective COVID-19 antiviral therapy that could shorten or prevent the course of the disease, reduce immune mediated pathological damage and lower disease severity is currently lacking. Nitric oxide has antimicrobial activity against bacteria, yeast, fungi, and viruses both in vitro and in vivo animal studies. NO also prevents the fusion between the SARS-CoV-2 Spike protein and its cognate receptor, ACE-2.

The current study was devised to determine the clinical efficacy of Nitric Oxide Nasal Spray (NONS) for the treatment of mild COVID-19 infection. The goal was to characterize nasal viral concentrations by quantitative real-time reverse-transcriptase polymerase chain reaction and establish the ability of treatment to clear the virus from the nasal cavity. The outcome measure was the difference in SARS-CoV-2 viral concentrations from baseline through Day 6 between NONS treatment and placebo. Eighty adults diagnosed with mild COVID-19 in the community were randomized in this double-blinded, placebo-controlled, phase IIb clinical trial.

NONS treatment started on or before day 5 of COVID-19 symptom onset was independently associated with an accelerated decrease in SARS-CoV-2 RNA concentration of −1.21 and −1.21 log₁₀ copies/mL on days 2 and 4 compared to placebo. Mean SARS-CoV-2 RNA concentration was lower on NONS treatment by a factor of 16.2 at both day 2 and 4. Day 6 equated to at least 10 days since symptom onset when a reduction of SARS-CoV-2 viral load with or without treatment was expected. Nevertheless, the SARS-CoV-2 RNA concentration was still lower on NONS treatment at day 6. The area under the curve analysis revealed a mean difference of −5.22 log₁₀ copies/mL with NONS treatment compared to placebo over the first 6 days of treatment. A rapid reduction (95%) in the high SARS-CoV-2 viral load occurred within 24 hours, and 99% reduction within 72 hours, on NONS treatments.

The majority of patients in this study were positive for the SARS-CoV-2 variant of concern (VOC202012_01). By comparison, a study of SARS-CoV-2 neutralizing antibody LY-CoV555 revealed a relative SARS-CoV-2 RNA concentration decrease versus placebo of −0.64 and −0.45 log₁₀ copies/mL at days 3 and 7, respectively. Oseltamivir has been shown to be independently associated with an influenza RNA concentration decrease of −1.19 and −0.68 log₁₀ copies/mL on days 2 and 4, respectively.

The reduction of SARS-CoV-2 viral load reduction in COVID-19 infected patients strongly correlates with better clinical outcomes, with viral load kinetics and duration of viral shedding being some determinants of disease transmission. Outcomes are best if viral load reduction begins within the first 5 days of symptom onset resulting in the lack of persistence of live viruses after 8-9 days, as demonstrated in monoclonal antibodies studies and vaccine trials using measured nasal viral load techniques comparable to the current trial.

Nasal deliver of NONS is effective in reducing COVID-19 viral load as are systemic therapies under investigation or currently being used to treat viral infections. Nitric oxide therapy is often overlooked as a COVID-19 treatment when potential beneficial treatments and pathogenic new insights are reviewed.

Overall, a difference in treatment across the efficacy and multiple secondary efficacy endpoints was observed between NONS compared to placebo. SARS-CoV-2 viral concentrations were immediately and consistently lowered with NONS treatment by a factor of 16.2-fold, suggesting a shortened COVID-19 infectivity duration in mild COVID-19 patients.

NONS could be used across the population for prevention or early treatment if new variants reduce the efficacy of the current vaccines. NONS could also provide antiviral treatment to those infected who may not yet have been fully vaccinated, those who are unable to be vaccinated or those with infection despite vaccination.

The 54 total nitric oxide nasal spray treatments, administered 6 times per day for 9 continuous days, with each treatment of 4 sprays (560 μL total), delivered as 2 sprays per nostril for a total dosage regimen of approximately 30 mL was well-tolerated in this Phase IIb efficacy and safety study. No new safety concerns were identified with this novel NO therapy (NONS) and its nasal administration to mild COVID-19 patients.

### EXAMPLE 4—Spray Testing

### SUMMARY

The testing involved three separate tests and was performed as follows:
Test Set 1: 6 total samples tested; Sample hand actuated at 30 mm distance from measurement zone.
Test Set 2: 6 total samples tested; Sample hand actuated at 60 mm distance from measurement zone.
Test Set 3: 2 samples tested (sample 1 and sample 4 were selected); Sample hand actuated at 30 mm distance from measurement zone; Samples tested by six total different individuals.

All Testing involved the following:
Data acquisition rate 500 Hz for is spray duration.
Data averaged from 0-250 ms to obtain fully developed spray data.
Refractive Index values of 1.33 and 0 were used.
All samples were primed and sprayed by hand throughout testing.

### Methods and Abbreviations:

The Malvern Spraytec was used for analysis and each spray was tested at 500 Hz for 1 second spray duration. The first 250 ms were averaged to identify the fully developed spray droplet size.

Beam Steering—Beam steering is a phenomenon in droplet size testing of aerosols where propellant causes false peaks of very large droplets to be apparently measured. From a measurement standpoint, this is caused by a change in the refractive index of the measurement zone. It is observed by the instrument's inner detectors. These detectors, when testing aerosols similar to the sample submitted, can be ignored in the analysis.

%<10 μm Reporting—The percentage of spray by volume at droplet sizes of 10 μm and less is thought to be inhalable and often requested in health and safety studies for consumer goods.

The following data include several size distribution values. The values are defined below:
Dv(10)—Indicative of the 10th percentile by volume of the spray.
Dv(50)—Indicative of the 50th percentile by volume of the spray. The median droplet size.
Dv(90)—Indication of the 90th percentile by volume of the spray.
Span—A calculation of breadth of distribution noted by (Dv(90)-Dv(10))/Dv(50).
% Volume <(um)—Measurement of the sample below a specific droplet size.
D[4,3]—Volume weighted mean of a spray.
D[3,2]—Surface weighted mean of a spray.

### Results:

TABLE 4-1

| | | | | % | % | |
|---|---|---|---|---|---|---|
| | Dv | Dv | Dv | V < | V < | |
| | (10) | (50) | (90) | 10μ | 5μ | Span |
| Sample 1 1 30 mm | 101.262 | 243.843 | 512.189 | 0.000 | 0.000 | 1.685 |
| Sample 1 2 30 mm | 140.994 | 355.327 | 693.430 | 0.000 | 0.000 | 1.555 |
| Sample 1 3 30 mm | 152.234 | 361.910 | 693.011 | 0.000 | 0.000 | 1.494 |
| Average | 131.497 | 320.360 | 632.877 | 0.000 | 0.000 | 1.578 |
| Sample 2 1 30 mm | 222.872 | 454.265 | 759.124 | 0.000 | 0.000 | 1.180 |
| Sample 2 2 30 mm | 221.623 | 457.970 | 767.876 | 0.000 | 0.000 | 1.193 |
| Sample 2 3 30 mm | 127.540 | 351.797 | 692.412 | 0.000 | 0.000 | 1.606 |
| Average | 190.678 | 421.344 | 739.804 | 0.000 | 0.000 | 1.326 |
| Sample 3 1 30 mm | 136.588 | 330.704 | 661.234 | 0.000 | 0.000 | 1.586 |
| Sample 3 2 30 mm | 130.490 | 324.886 | 656.586 | 0.000 | 0.000 | 1.619 |
| Sample 3 3 30 mm | 127.612 | 327.289 | 660.988 | 0.000 | 0.000 | 1.630 |
| Average | 131.563 | 327.626 | 659.603 | 0.000 | 0.000 | 1.612 |
| 30 mm Average | 151.246 | 356.443 | 677.428 | 0.000 | 0.000 | 1.505 |

TABLE 4-2

| | | | | % | % | |
|---|---|---|---|---|---|---|
| | Dv | Dv | Dv | V < | V < | |
| | (10) | (50) | (90) | 10μ | 5μ | Span |
| Sample 4 1 30 mm | 122.029 | 301.719 | 614.164 | 0.000 | 0.000 | 1.631 |
| Sample 4 2 30 mm | 142.992 | 330.202 | 656.950 | 0.000 | 0.000 | 1.556 |
| Sample 4 3 30 mm | 154.370 | 330.690 | 647.151 | 0.000 | 0.000 | 1.490 |
| Average | 139.797 | 320.870 | 639.422 | 0.000 | 0.000 | 1.559 |
| Sample 5 1 30 mm | 394.825 | 587.198 | 795.017 | 0.000 | 0.000 | 0.682 |
| Sample 5 2 30 mm | 392.598 | 578.568 | 801.787 | 0.147 | 0.022 | 0.707 |
| Sample 5 3 30 mm | 366.546 | 558.278 | 797.681 | 0.133 | 0.026 | 0.772 |
| Average | 384.656 | 574.681 | 798.162 | 0.093 | 0.016 | 0.720 |
| Sample 6 1 30 mm | 243.500 | 455.451 | 755.612 | 0.000 | 0.000 | 1.124 |
| Sample 6 2 30 mm | 275.587 | 481.120 | 759.873 | 0.000 | 0.000 | 1.007 |
| Sample 6 3 30 mm | 246.835 | 447.999 | 739.645 | 0.000 | 0.000 | 1.100 |
| Average | 255.307 | 461.523 | 751.710 | 0.000 | 0.000 | 1.077 |
| 30 mm Average | 259.920 | 452.358 | 729.764 | 0.031 | 0.005 | 1.119 |

TABLE 4-3

| | | | | % | % | |
|---|---|---|---|---|---|---|
| | Dv | Dv | Dv | V < | V < | |
| | (10) | (50) | 90) | 10μ | 5μ | Span |
| Sample 1 1 60 mm | 152.514 | 386.281 | 714.601 | 0.000 | 0.000 | 1.455 |
| Sample 1 2 60 mm | 107.093 | 347.103 | 690.122 | 0.000 | 0.000 | 1.680 |
| Sample 1 3 60 mm | 109.096 | 339.090 | 679.544 | 0.000 | 0.000 | 1.682 |
| Average | 122.901 | 357.491 | 694.756 | 0.000 | 0.000 | 1.606 |
| Sample 2 1 60 mm | 72.467 | 340.656 | 698.964 | 0.000 | 0.000 | 1.839 |
| Sample 2 2 60 mm | 77.487 | 381.818 | 725.977 | 0.000 | 0.000 | 1.698 |
| Sample 2 3 60 mm | 67.666 | 352.255 | 708.501 | 0.000 | 0.000 | 1.819 |
| Average | 72.540 | 358.243 | 711.148 | 0.000 | 0.000 | 1.786 |
| Sample 3 1 60 mm | 75.445 | 249.704 | 561.301 | 0.000 | 0.000 | 1.946 |
| Sample 3 2 60 mm | 89.285 | 341.403 | 679.405 | 0.000 | 0.000 | 1.729 |
| Sample 3 3 60 mm | 84.185 | 325.247 | 673.094 | 0.000 | 0.000 | 1.811 |
| Average | 82.972 | 305.451 | 637.933 | 0.000 | 0.000 | 1.828 |
| 60 mm Average | 92.804 | 340.395 | 681.279 | 0.000 | 0.000 | 1.740 |

TABLE 4-4

| | | | | % | % | |
|---|---|---|---|---|---|---|
| | Dv | Dv | Dv | V < | V < | |
| | (10) | (50) | (90) | 10μ | 5μ | Span |
| Sample 4 1 60 mm | 140.840 | 336.082 | 659.939 | 0.000 | 0.000 | 1.545 |
| Sample 4 2 60 mm | 143.321 | 343.788 | 667.306 | 0.000 | 0.000 | 1.524 |
| Sample 4 3 60 mm | 154.273 | 359.728 | 688.211 | 0.000 | 0.000 | 1.484 |
| Average | 146.144 | 346.533 | 671.818 | 0.000 | 0.000 | 1.518 |
| Sample 5 1 60 mm | 379.650 | 577.786 | 812.706 | 0.146 | 0.030 | 0.750 |
| Sample 5 2 60 mm | 432.757 | 584.474 | 764.496 | 0.000 | 0.000 | 0.568 |
| Sample 5 3 60 mm | 417.434 | 582.508 | 777.061 | 0.000 | 0.000 | 0.617 |
| Average | 409.947 | 581.589 | 784.755 | 0.049 | 0.010 | 0.645 |
| Sample 6 1 60 mm | 213.869 | 434.029 | 741.156 | 0.000 | 0.000 | 1.215 |
| Sample 6 2 60 mm | 226.458 | 452.035 | 754.750 | 0.000 | 0.000 | 1.169 |
| Sample 6 3 60 mm | 224.597 | 449.448 | 752.489 | 0.000 | 0.000 | 1.175 |
| Average | 221.641 | 445.171 | 749.465 | 0.000 | 0.000 | 1.186 |
| 60 mm Average | 259.244 | 457.764 | 735.346 | 0.016 | 0.003 | 1.116 |

TABLE 4-5

| | | | | % | % | |
|---|---|---|---|---|---|---|
| | Dv | Dv | Dv | V < | V < | |
| | (10) | (50) | (90) | 10μ | 5μ | Span |
| Sample 1 1 30 mm | 101.262 | 243.843 | 512.189 | 0.000 | 0.000 | 1.685 |
| Sample 1 2 30 mm | 140.994 | 355.327 | 693.430 | 0.000 | 0.000 | 1.555 |
| Sample 1 3 30 mm | 152.234 | 361.910 | 693.011 | 0.000 | 0.000 | 1.494 |
| Average | 131.497 | 320.360 | 632.877 | 0.000 | 0.000 | 1.578 |
| Sample 1 Person 2 1 | 155.124 | 330.574 | 641.942 | 0.000 | 0.000 | 1.473 |
| Sample 1 Person 2 2 | 111.973 | 249.584 | 505.732 | 0.000 | 0.000 | 1.578 |
| Sample 1 Person 2 3 | 117.379 | 270.817 | 552.242 | 0.000 | 0.000 | 1.606 |
| Average | 128.159 | 283.658 | 566.639 | 0.000 | 0.000 | 1.552 |
| Sample 1 Person 3 1 | 146.181 | 321.294 | 635.651 | 0.000 | 0.000 | 1.523 |
| Sample 1 Person 3 2 | 140.653 | 292.301 | 574.648 | 0.000 | 0.000 | 1.485 |
| Sample 1 Person 3 3 | 127.068 | 271.158 | 525.124 | 0.000 | 0.000 | 1.468 |
| Average | 137.968 | 294.918 | 578.474 | 0.000 | 0.000 | 1.492 |
| Sample 1 Person 4 1 | 127.310 | 282.311 | 567.591 | 0.000 | 0.000 | 1.560 |
| Sample 1 Person 4 2 | 129.144 | 288.962 | 578.900 | 0.000 | 0.000 | 1.556 |
| Sample 1 Person 4 3 | 119.532 | 276.141 | 563.163 | 0.000 | 0.000 | 1.607 |
| Average | 125.328 | 282.471 | 569.885 | 0.000 | 0.000 | 1.574 |
| Sample 1 Person 5 1 | 115.992 | 275.030 | 545.491 | 0.000 | 0.000 | 1.562 |
| Sample 1 Person 5 2 | 149.494 | 303.484 | 592.519 | 0.000 | 0.000 | 1.460 |
| Sample 1 Person 5 3 | 136.821 | 295.233 | 588.674 | 0.000 | 0.000 | 1.530 |
| Average | 134.102 | 291.249 | 575.561 | 0.000 | 0.000 | 1.517 |
| Sample 1 Person 6 1 | 108.952 | 285.052 | 593.417 | 0.000 | 0.000 | 1.700 |
| Sample 1 Person 6 2 | 90.469 | 253.564 | 526.969 | 0.000 | 0.000 | 1.721 |
| Sample 1 Person 6 3 | 97.869 | 266.085 | 561.405 | 0.000 | 0.000 | 1.742 |
| Average | 99.096 | 268.234 | 560.597 | 0.000 | 0.000 | 1.721 |
| Overall Average | 126.025 | 290.148 | 580.672 | 0.000 | 0.000 | 1.572 |

TABLE 4-6

| | | | | % | % | |
|---|---|---|---|---|---|---|
| | Dv | Dv | Dv | V < | V < | |
| | (10) | (50) | (90) | 10μ | 5μ | Span |
| Sample 4 1 30 mm | 122.029 | 301.719 | 614.164 | 0.000 | 0.000 | 1.631 |
| Sample 4 2 30 mm | 142.992 | 330.202 | 656.950 | 0.000 | 0.000 | 1.556 |
| Sample 4 3 30 mm | 154.370 | 330.690 | 647.151 | 0.000 | 0.000 | 1.490 |
| Average | 139.797 | 320.870 | 639.422 | 0.000 | 0.000 | 1.559 |
| Sample 4 Person 2 1 | 172.016 | 334.082 | 632.883 | 0.000 | 0.000 | 1.380 |
| Sample 4 Person 2 2 | 158.809 | 317.066 | 613.892 | 0.000 | 0.000 | 1.435 |
| Sample 4 Person 2 3 | 151.897 | 300.449 | 582.810 | 0.000 | 0.000 | 1.434 |
| Average | 160.907 | 317.199 | 609.862 | 0.000 | 0.000 | 1.416 |
| Sample 4 Person 3 1 | 156.337 | 338.023 | 653.529 | 0.000 | 0.000 | 1.471 |
| Sample 4 Person 3 2 | 134.414 | 317.845 | 651.932 | 0.000 | 0.000 | 1.628 |
| Sample 4 Person 3 3 | 135.615 | 336.450 | 671.657 | 0.000 | 0.000 | 1.593 |
| Average | 142.122 | 330.773 | 659.040 | 0.000 | 0.000 | 1.564 |
| Sample 4 Person 4 1 | 157.070 | 334.560 | 644.057 | 0.000 | 0.000 | 1.456 |
| Sample 4 Person 4 2 | 159.722 | 325.183 | 628.428 | 0.000 | 0.000 | 1.441 |
| Sample 4 Person 4 3 | 139.143 | 299.934 | 592.070 | 0.000 | 0.000 | 1.510 |
| Average | 151.979 | 319.892 | 621.518 | 0.000 | 0.000 | 1.469 |
| Sample 4 Person 5 1 | 168.296 | 345.293 | 657.279 | 0.000 | 0.000 | 1.416 |
| Sample 4 Person 5 2 | 170.439 | 348.169 | 661.881 | 0.000 | 0.000 | 1.412 |
| Sample 4 Person 5 3 | 186.346 | 341.521 | 653.906 | 0.000 | 0.000 | 1.428 |
| Average | 168.361 | 344.994 | 657.689 | 0.000 | 0.000 | 1.418 |
| Sample 4 Person 6 1 | 170.411 | 340.558 | 646.722 | 0.000 | 0.000 | 1.399 |
| Sample 4 Person 6 2 | 159.569 | 327.904 | 633.138 | 0.000 | 0.000 | 1.444 |
| Sample 4 Person 6 3 | 142.897 | 321.273 | 643.189 | 0.000 | 0.000 | 1.557 |
| Average | 157.626 | 329.912 | 641.017 | 0.000 | 0.000 | 1.467 |
| Overall Average | 153.465 | 327.273 | 638.091 | 0.000 | 0.000 | 1.482 |

### Discussion:

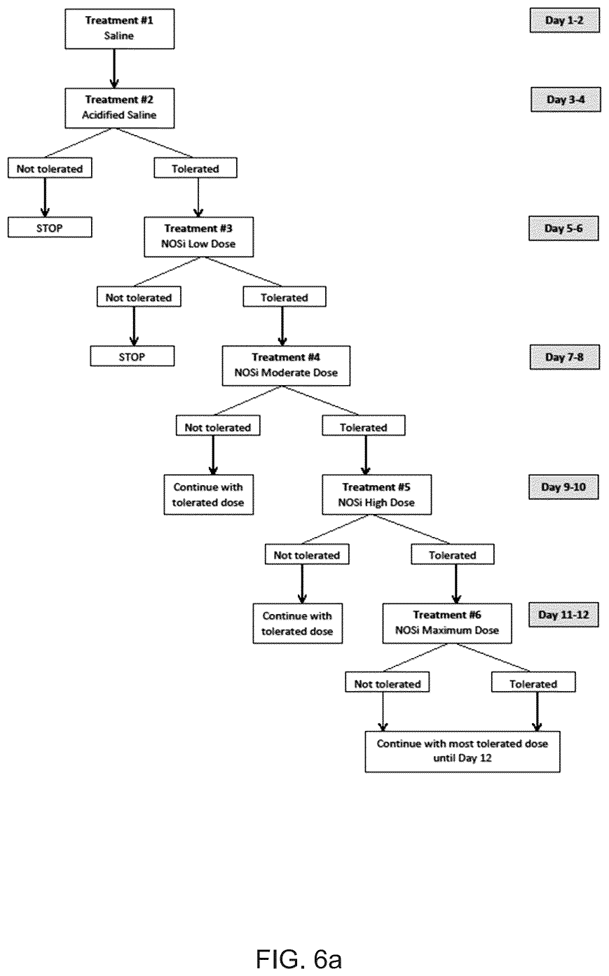

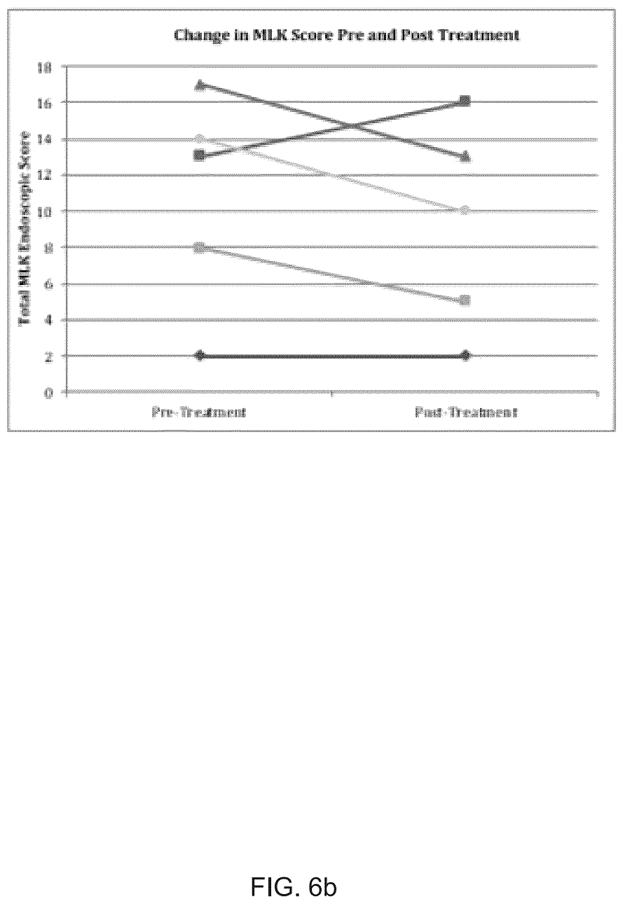

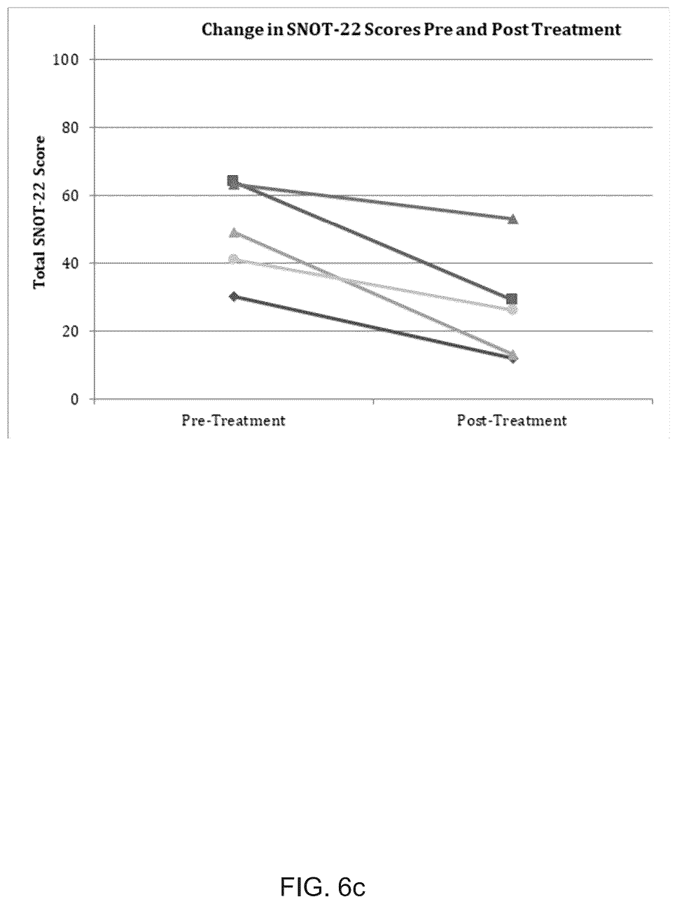

30 mm Data for Triplicate Analysis and Sets of Three Samples. As depicted in FIGS. 6a and 6b, the overlay plots for the triplicate analysis of each individual sample with all samples represented. The goal of these plots is to assess consistency both within a sample bottle as well as from sample to sample. In this case, the same individual sprayed each sample and did so in a consistent manner. The first 250 ms was determined to be the stable fully developed phase of the spray throughout the testing. This was utilized in all of the data averaging to ensure the spray is most properly characterized.

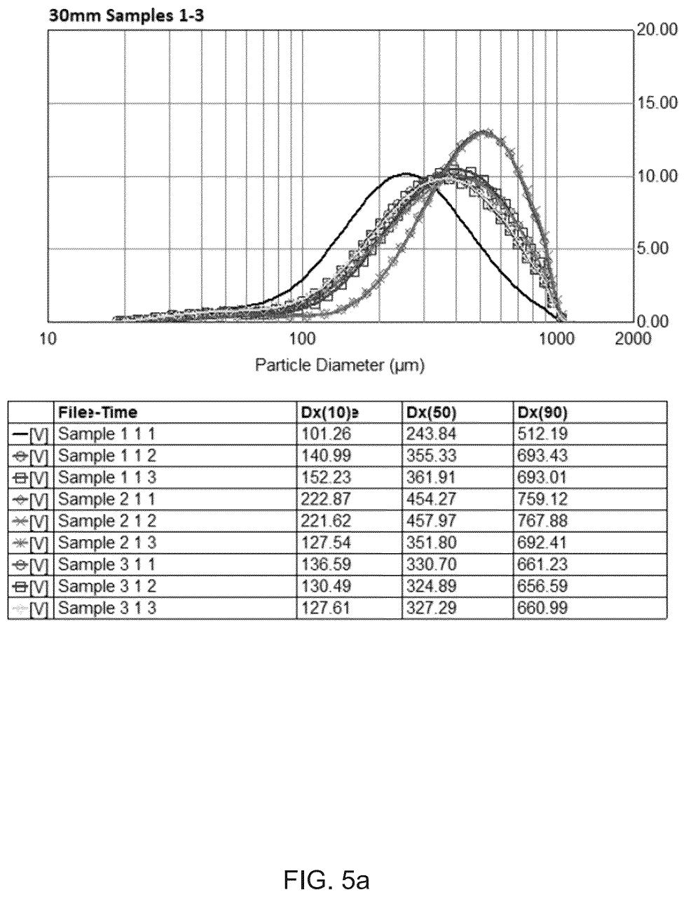

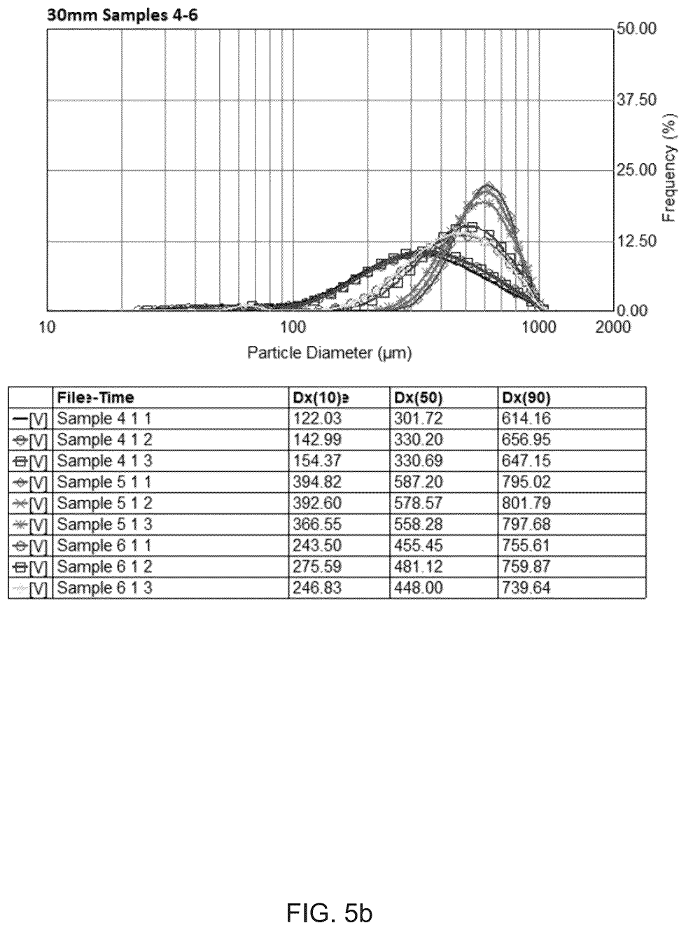

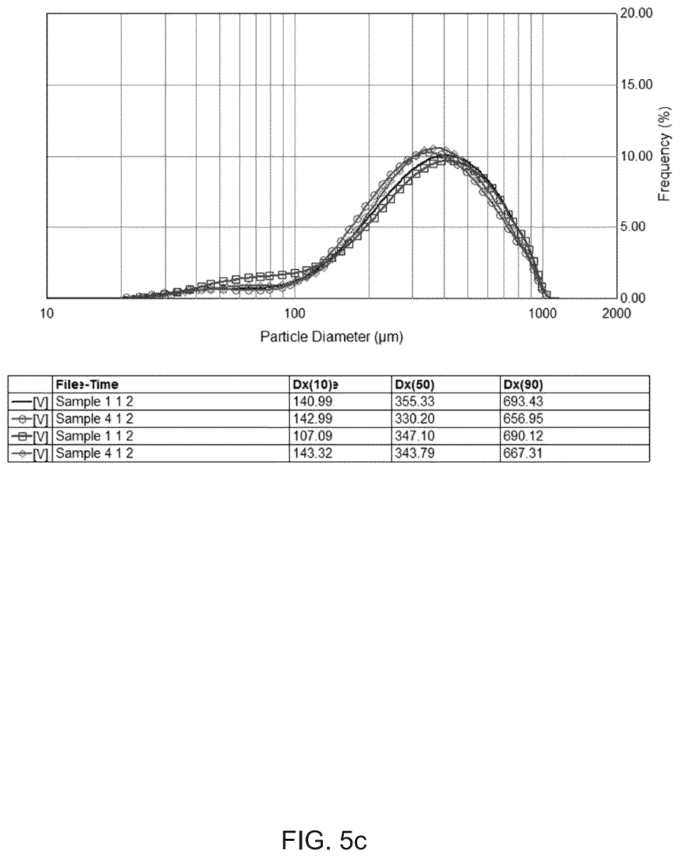

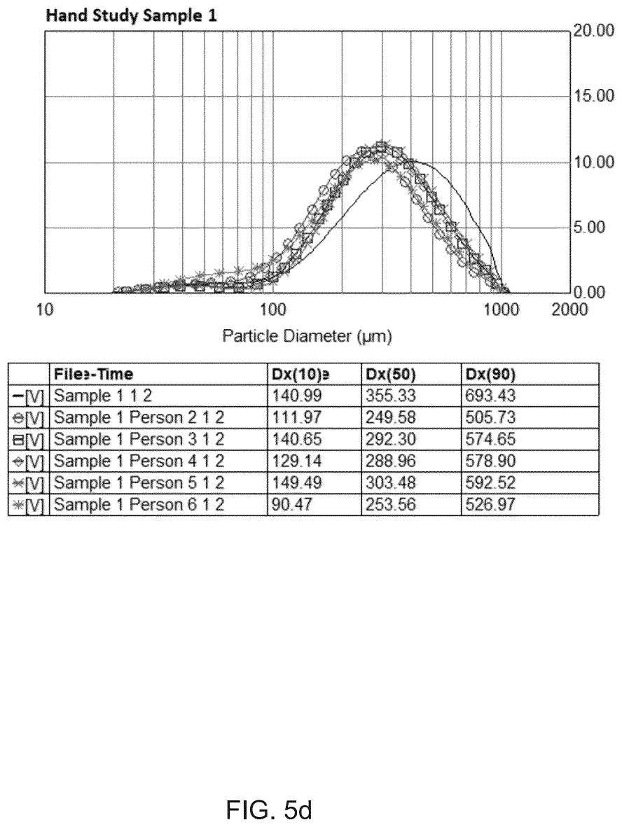

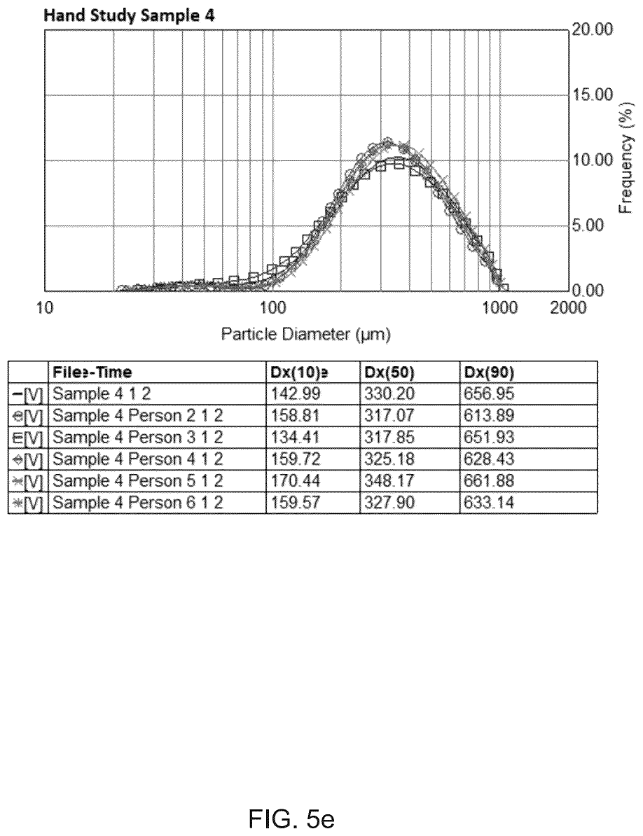

As depicted in FIG. 5a, the results of samples 1-3 are shown. There appears to be a fairly consistent plot trace in the center of the plot with a few outlier plot traces. Sample 2 was found to be larger than the other plots in terms of droplet size. This plot was used to determine sample 1 would be used in the hand study to be conducted with several different operators. Overall, the differences involved in this plot may be significant and indicate a difference from bottle to bottle in terms of output. The droplet size distributions output were large in comparison with the traditional nasal delivery systems.

As depicted in FIG. 5b, the results of samples 4-6 are shown. Sample 5 specifically exhibited no atomization and instead just a stream of material being output. The reported results of sample 5 were governed by the top size range of the instrument as opposed to the actual spray. The other two samples show some difference as well. Sample 4 was selected for the hand study due to its similarity to Sample 1 results at the same testing.

The 60 mm results also mimic the results above and the data can be seen on the tables on the prior page tables.

### Sample 1 and Sample 4 at 30 mm and 60 mm

As depicted in FIG. 5c, four total tests are plotted. As depicted in FIG. 5c, Sample 1 and Sample 4 each at 30 mm distance. As depicted in FIG. 5c, Sample 1 and Sample 4 each at 60 mm.

As depicted in FIG. 5c, there similarity between the two samples tested by the same individual. The output at the two distances is nearly identical. This was expected as the size output of the devices is quite large. With that, the full spray is more likely to make it to both distances versus if smaller droplets are involved which may not have the momentum to travel the distances.

As depicted in FIG. 5c, the two samples as very similar making them a reasonable choice for a hand study to compare the outputs with different users.

### Hand Study Results and Information

A hand study was conducted with six total users to emulate a range of users with the device. The users could be classified as follows:
User 1 Male 35-49
User 2 Male 60+
User 3 Male 35-49
User 4 Male 11-19
User 5 Female 11-19
User 6 Female 35-49

The users above all sprayed samples 1 and 4 three times at the 30 mm distance in the same manner as was done in the prior testing plan.

As depicted in FIGS. 6d and 6e, there is variability of the two samples during the hand study. This shows that under normal usage, the bottle performs fairly similarly regardless of the person spraying. This would indicate the packaging is able to output a similar droplet size across a wider user base and that the results could be replicated with an automatic actuator system. There appears to be a wider range of droplet size output from bottle to bottle than there is from user to user.

### Spray Weights:

The spray weights per dose were found to be consistently in the 0.13-0.15 g for all bottles. Sample 5 was on the lower end of this consistently but the other samples were all in this same range throughout the testing.

### Conclusion:

The droplet size output in general of this device is fairly large in terms of droplet size. There was almost no material under 5 μm and 10 μm respectively. The atomization of the material was minimal leaving large droplets. In one bottle, sample 5, there was no atomization at all present and instead just a stream of material coming out of the spray tip.

In terms of repeatability, the consistency within a specific bottle, this was found to be fairly high. Once the sample was primed, the triplicate analysis of that bottle was for the most part consistent. This indicates that the bottles are most likely repriming and outputting a similar droplet size distribution during testing of that sample.

In terms of reproducibility, the consistency from bottle to bottle, both sets of samples displayed some inconsistency. This was clear at both distances and was clear in the overlay plots provided. This suggests some differences from bottle to bottle that is being shown by the droplet size distribution. While an automatic actuator may help to lessen the differences, the differences would be expected to be present under any testing procedure given the wide ranges experienced.

The hand study showed that the same sample sprayed by several different people and force profiles produced a small variance in droplet size. This suggests that the user of the device was not that determinative in terms of droplet size output. Instead, the physical device itself appears to have more variability at this time. That is, the difference in droplet size appears to be related to the bottle and pump itself much more than the user of the device.

The spray output for each bottle was very similar to the other bottles. One bottle to be slightly different was bottle 5 which sprayed a stream and had no atomization to it. Those sprays were on the lower spray weight side than the other bottles. The range was roughly 0.13-0.15 g per spray.

### EXAMPLE 5—Topical Nitric Oxide Sinus Irrigation

### Overview:

Recalcitrant chronic rhinosinusitis (RCRS) is a persistent inflammatory condition despite surgery and aggressive medical therapies. Nitric Oxide (NO) is an endogenously produced molecule that exhibits antimicrobial & anti-inflammatory properties. This study aimed to determine the tolerance and safety of escalating dose treatments of NO sinus irrigation (NOSi) in RCRS adults.

5 adult subjects with RCRS irrigated their sinuses twice daily for 12 days with NOSi with dose escalation every 2 days. Safety monitoring on days 3, 5, 7, 9 and 11 included tolerability as reported by Visual Analogue Scale (VAS), adverse events (AE), methemoglobin (MetHb), O₂ saturation (SaO₂) and ambient NO₂. Changes to Modified Lund-Kennedy (MLK) endoscopic score, sino-nasal mucosal culture, olfaction, mucociliary function, and quality of life as measured by Sino-Nasal Outcome Test (SNOT-22) were recorded at baseline and day 13.

⅘ subjects tolerated the highest dose of NOSi twice daily. No AE or changes to ambient NO₂, MetHb, or SaO₂ outside of normal range were reported. ⅗ subjects exhibited improvements in total MLK score (baseline median=13, mean=9.25; day 13 median=10, mean=9.2). Reduced growth of bacterial & fungal organisms was reported in ⅗ subjects. SNOT-22 score improved in all subjects (baseline median=49, mean=49.4; day 13 median=26, mean=26.6). Increases in mucociliary clearance time within normal ranges were noted in ⅗ subjects. No significant changes to olfaction or mucosal tissue were reported. Preliminary results suggest NOSi is a tolerable and safe sinus irrigation and could provide an efficacious treatment for RCRS.

### Methods:

Five (n=5) adult subjects aged 19 years or older and diagnosed with recalcitrant CRS with biofilm were included. Recalcitrant CRS was defined as continued symptoms of colored nasal discharge, post-nasal drip, nasal congestion, decreased sense of smell, and mucosal edema as indicated on nasal endoscopy for at least 3 months despite appropriate medical therapy including topical irrigation with corticosteroids and well-executed endoscopic sinus surgery. Individuals who presented with sino-nasal tumors, nasal polyps, autoimmune disorders with sino-nasal manifestations, pregnancy, history or presence of cardiovascular disease, stroke history, drug use that could induce methemoglobinemia or had used any investigational drug within the past 30 days were excluded from the study. All participants were asked to undergo a wash-out period of 30 days if using concurrent medications that may improve sinus symptoms (betadine irrigation, topical antibiotic treatment, systemic steroids etc.).

Demographic information and history of comorbidities were collected from all participants. Participants were instructed on how to prepare and administer NOSi in saline by self-administration as a sinus irrigation to their nasopharynx and sinuses twice daily (at least 6 hours apart) for 12 days. Doses included: saline, acidified saline, “low”, “moderate”, “high”, and “maximum” NOSi dose. NOSi dose escalations occurred every two days on days 3, 5, 7, 9 and 11 with research staff in clinic, as shown in FIG. 6a. Participants were blinded to the dosage of the irrigation; however, were made aware that they will receive increasing NOSi dosages throughout the 12 day period. Safety and tolerability monitoring occurred throughout the study duration whereas efficacy evaluation occurred at baseline and Day 13.

Tolerability was evaluated using patient reported visual analogue scores (VAS) for pain and discomfort after each treatment. The VAS included five 10 cm horizontal lines and participants were instructed to evaluate prickling, stinging, throbbing, cramping and stabbing pain post-irrigation. A total VAS score out of 50 including all pain descriptors was used to evaluate patient reported tolerability. Additionally, tolerability was determined by the participant's willingness to tolerate treatments of a maximum of two or minimum of one NOSi treatments for up to 6 consecutive days.

Safety was evaluated by close monitoring of methemoglobin, oxygen saturation and environmental NO₂ levels because of the known potential for NO/NO₂ to impact these parameters if exposed to vascular surfaces; as well adverse events were recorded. Vital signs, including blood pressure, respiratory rate and heart rate were measured pre- and post-each dose escalation in clinic. Ciliary function was measured by the saccharin time test and histopathology test. Olfaction was measured using the University of Pennsylvania Smell Identification Test (UPSIT). Changes in these safety values and adverse events are customary measures of safety.

Efficacy was evaluated by performing routine endoscopy and assessed with the modified Lund Kennedy (MLK) score for chronic rhinosinusitis. Bacterial load in the sinuses was obtained with swab samples sent to the laboratory for semi-quantitative culturing & sensitivity. Patient reported quality of life was assessed using the validated disease-specific Sinonasal Outcomes Test (SNOT-22). This clinical evaluation was intended to identify tolerance of NOSi in this patient population. Mean values and standard deviations were reported.

### Results:

5 patients were enrolled in the proof-of-concept study. Patient demographics and characteristics are summarized in Table 5-1.

TABLE 5-1
Summary of demographic of study population

| Characteristic | Result |
|---|---|
| Mean Age (SD) | 64.4 (±6.09) |
| Gender | |
| Male | 80% |
| Female | 20% |
| Ethnicity | |
| Caucasian | 100% |
| Smoking Status | |
| Current Smoker | 0% |
| Former Smoker | 40% |
| Non-Smoker | 60% |

Four of five (80%) subjects tolerated the “maximum” dose of NOSi twice daily. Due to a protocol amendment, one participant did not trial the “low” dose. This same patient was unable to tolerate the “high” dose and thus, irrigated with “moderate” dose of NOSi once daily for the remainder of the study. Participants reported a mean 2.53, 3.98, 2.53, and 3.32 point increase in total pain and discomfort between “low”, “moderate”, “high” and “maximum” doses, respectively, compared to saline, as shown in Table 5-2. In general, participants reported stinging and prickling pain in the nostrils that occurred immediately when rinsing with NOSi and resolved throughout the irrigation.

TABLE 2
Mean Visual Analogue Scale (VAS) scores
reported immediately post-irrigation

| | Mean Total | |
|---|---|---|
| Dose | VAS Score (SD) | |
| Saline | 0.1 | (SD) |
| Acidified Saline | 0.1 | (SD) |
| “Low” NOSi (n = 4) | 2.53 | (2.43) |
| “Moderate” NOSi (n = 5) | 3.98 | (3.88) |
| “High” NOSi (n = 4) | 2.53 | (2.43) |
| “Maximum” NOSi (n = 4) | 3.32 | (3.22) |

No significant changes to olfaction were reported in four patients (80%) whereas one participant (200%) experienced a clinically significant decrease of 5 points between baseline pre and post NOSi treatment on day 13. No significant changes to mucosal tissue including, presence of vasculitis, necrosis, or dysplasia, were observed. Increases in mucociliary clearance time within normal ranges were noted in three of five (60%) subjects. No adverse events were reported by any participant. Furthermore, there were no events of ambient NO₂ increasing greater than 5 ppm reported. Increases in methemoglobin values within pre and post NOSi treatments varied between; however, all changes remained under 1.5%. Furthermore, no changes to 02 saturation outside of normal range were measured during treatment with NOSi. Post treatment vital signs (blood pressure, respiratory rate, heart rate) remained within normal range in all participants.

Three participants (60%) exhibited a mean improvement in total Modified Lund-Kennedy score of 3.33 points (baseline median=13, mean=9.25;day 13 median=10, mean=9.2). 1 participant (20%) experienced no change in MLK and 1 participant experienced a 3 point decrease, as shown in FIG. 6b. Reduced growth of bacterial & fungal organisms was reported in three of five (60%) subjects. Two patients (40%) demonstrated complete reduction of light or moderate growth of Staphylococcus aureus. One patient (20%) exhibited a complete reduction of fungal organism, Exophiala dermatitidis. Two patients (40%) either did not exhibit a change in bacterial growth or experienced a new fungal growth of Aspergillus niger complex after the 13-day study period. All (100%) patients demonstrated an improvement of at least one minimally clinically detectable difference (1 MCID=8.9 points) in their SNOT-22 scores with a mean improvement of 23.6 points (pre-treatment median=49, mean=49.4; post-treatment median=26, mean=26.6), as shown in FIG. 6c.

### Discussion:

The overarching goal of recalcitrant CRS management is to reduce mucosal edema, eradicate biofilm formation, and ultimately, improve patient outcomes. With the current lack of a safe, effective and long-term antimicrobial therapy, this study aimed to evaluate the safety and tolerability of Nitric Oxide sinus irrigation for treatment of recalcitrant chronic rhinosinusitis.

The purpose of this pilot study was to determine the tolerability and safety of nitric oxide sinus irrigation. To our knowledge, there are no studies that have previously examined the safety or efficacy of a topical nitric oxide sinus irrigation. Our preliminary results have identified a tolerable dose of NOSi for further safety and efficacy evaluation in a larger, double-blind, randomized controlled trial.

Our results indicate that 80% of participants tolerated the “maximum” dose of NOSi. One participant reported prickling and stinging pain greater than 5 (out of 10) for the “maximum” dose which resolved immediately after completing the irrigation. No adverse events were noted in this study including, ambient nitrogen dioxide levels of greater than 5 ppm or methemeglobin change of greater than 5%. Interestingly, one participant demonstrated a clinically significant decrease in olfaction compared to baseline which contradicts present literature suggesting that neuronal nitric oxide synthase is highly expressed in the mammalian olfactory bulb and involved in olfactory processing. This contradictory preliminary finding provides rationale for the inclusion of further olfactory measures in the randomized controlled trial.

One participant demonstrated novel fungal growth over the course of the study; however, this was the same participant who was unable to tolerate the “moderate” dose and irrigated with the “low” dose for the majority of the study. Two participants demonstrated a partial reduction in bacterial sino-nasal culture growth while one participant demonstrated a complete reduction in fungal growth. These preliminary results suggest that NOSi could be efficacious against both bacterial and fungal biofilms and thus, could be efficacious as a long-term recalcitrant CRS treatment. Furthermore, upon endoscopic examination, three participants indicated an overall improvement in MLK score while, one participant indicated no change and one deteriorated. Interestingly, the participant whose MLK score worsened reported a 35-point improvement in SNOT-22 questionnaire.

### Conclusion:

This prospective pilot study provided preliminary data of NOSi dose tolerance and short-term safety. This study provides a concrete base for the initiation of a phase II randomized controlled trial that will evaluate the efficacy and long-term safety of NOSi.

It is understood that the above-described various types of compositions, dosage forms and/or modes of applications are only illustrative of embodiments of the present invention. Numerous modifications and alternative arrangements may be devised by those skilled in the art without departing from the spirit and scope of the present invention and the appended claims are intended to cover such modifications and arrangements. Thus, while the present invention has been described above with particularity and detail in connection with what is presently deemed to be the most practical and embodiments of the invention, it will be apparent to those of ordinary skill in the art that variations including, but not limited to, variations in size, materials, shape, form, function and manner of operation, assembly and use may be made without departing from the principles and concepts set forth herein.

1-23. (canceled)

24. A method of minimizing subject-to-subject transmissibility for a pathogen comprising:
administering a therapeutically effective amount of a nitric oxide releasing solution (NORS) to the subject.

25. The method of claim 24, further comprising:
administering the NORS to the subject before or after exposure of the subject to the pathogen.

26. The method of claim 24, further comprising:
administering the NORS to the subject before or after exposure of the subject to a person displaying symptoms of the pathogen.

27. The method of claim 24, wherein the pathogen is a Severe Acute Respiratory Syndrome Coronavirus (SARSr-CoV) or a variant thereof.

28. The method of claim 27, wherein the Severe Acute Respiratory Syndrome Coronavirus (SARSr-CoV) is a SARS-CoV-2 virus including one or more of: Lineage A, Cluster 5, Lineage B.1.1.7, B.1.1.7 with E484K, Lineage B.1.1.207, Lineage B.1.1.317, Lineage B.1.1.318, Lineage B.1.351, Lineage B.1.429/CAL.20C, Lineage B.1.525, Lineage P.1, Lineage B.1.427, Lineage B.1.526, Lineage P.2, or a combination thereof.

29. The method of claim 24, wherein the therapeutically effective amount of the NORS is substantially equally effective for minimizing subject-to-subject transmissibility for a a Severe Acute Respiratory Syndrome Coronavirus (SARSr-CoV) or a variant thereof including one or more of: Lineage A, Cluster 5, Lineage B.1.1.7, B.1.1.7 with E484K, Lineage B.1.1.207, Lineage B.1.1.317, Lineage B.1.1.318, Lineage B.1.351, Lineage B.1.429/CAL.20C, Lineage B.1.525, Lineage P.1, Lineage B.1.427, Lineage B.1.526, Lineage P.2, or a combination thereof.

30. The method of claim 24, wherein the therapeutically effective amount of the NORS reduces a viral RNA load compared to a baseline level by greater than one or more of: 80%, 90%, 95%, or 99% after a treatment duration of one or more of: 1 day, 2 days, 3 days, 4 days, 5 days, or 6 days.

31. The method of claim 24, wherein the therapeutically effective amount of the NORS:
provides a low pH that reduces SARS-CoV-2 viral load, or
provides a physical barrier over epithelial cells of the subject that prevents SARS-CoV-2 access to host cells of the subject, or
provides an angiotensin-converting enzyme 2 (ACE-2) receptor blocking agent.

32. The method of claim 24, wherein the NORS is administered to a mucosal membrane.

33. The method of claim 32, wherein the mucosal membrane is a subject's nasal passage or sinus and the NORS is administered as a sprayed solution or as a lavage.

34. The method of claim 32, wherein the mucosal membrane is a subject's mouth or throat and the NORS is administered as a gargle solution.

35. A method of treating a subject for infection of a pathogen in an upper respiratory tract comprising:
administering a therapeutically effective amount of a nitric oxide releasing solution (NORS) to the subject as a spray having an average droplet volume which contains treatment within the upper respiratory tract.

36. The method of claim 35, further comprising:
providing a median droplet size (Dv50) of greater than one or more of 100 μm, 150 μm, 200 μm, 250 μm, 300 μm, 350 μm, 400 μm, 450 μm, 500 μm, 550 μm, or 600 μm when measured a 30 mm or 60 mm distance from actuation.

37. The method of claim 35, further comprising:
providing a percentage of spray by volume at droplet sizes of less than 10 μm (%<10 μm) of less than one or more of: 0.5%, 0.4%, 0.3%, 0.2%, or 0.1% when measured a 30 mm or 60 mm distance from actuation.

38. The method of claim 35, further comprising:
providing a percentage of spray by volume at droplet sizes of less than 5 μm (%<5 μm) of less than one or more of: 0.5%, 0.4%, 0.3%, 0.2%, or 0.1% when measured a 30 mm or 60 mm distance from actuation.

39. The method of claim 35, further comprising:
providing a 10^{th} percentile by volume of spray (Dv(10)) of greater than one or more of 100 μm, 150 μm, 200 μm, 250 μm, 300 μm, 350 μm, or 400 μm when measured a 30 mm or 60 mm distance from actuation.

40. The method of claim 35, further comprising:
providing a 90^{th} percentile by volume of spray (Dv(90)) of greater than one or more of 500 μm, 550 μm, 600 μm, 650 μm, 700 μm, 750 μm, 800 μm, 850 μm, 900 μm, 950 μm, or 1000 μm when measured a 30 mm or 60 mm distance from actuation.

41. The method of claim 35, further comprising:
providing a droplet size distribution of about 0.5 to about 2.0, wherein the droplet size distribution is: (Dv(90)−Dv(10))/Dv(50).

42. A nitric oxide releasing solution (NORS) comprising:
at least one nitric oxide releasing compound and at least one acidifying agent, wherein the NORS releases a therapeutically effective amount as a spray having an average droplet volume which contains treatment within an upper respiratory tract.

43. The solution of claim 42, wherein the NORS provides a median droplet size (Dv50) of greater than one or more of 100 μm, 150 μm, 200 μm, 250 μm, 300 μm, 350 μm, 400 μm, 450 μm, 500 μm, 550 μm, or 600 μm when measured a 30 mm or 60 mm distance from actuation.

44. The solution of claim 42, wherein the NORS provides a percentage of spray by volume at droplet sizes of less than 10 μm (%<10 μm) of less than one or more of: 0.5%, 0.4%, 0.3%, 0.2%, or 0.1% when measured a 30 mm or 60 mm distance from actuation.

45. The solution of claim 42, wherein the NORS provides a percentage of spray by volume at droplet sizes of less than 5 μm (%<5 μm) of less than one or more of: 0.5%, 0.4%, 0.3%, 0.2%, or 0.1% when measured a 30 mm or 60 mm distance from actuation.

46. The solution of claim 42, wherein the NORS provides a 10^{th} percentile by volume of spray (Dv(10)) of greater than one or more of 100 μm, 150 μm, 200 μm, 250 μm, 300 μm, 350 μm, or 400 μm when measured a 30 mm or 60 mm distance from actuation.

47. The solution of claim 42, wherein the NORS provides a 90^{th} percentile by volume of spray (Dv(90)) of greater than one or more of 500 μm, 550 μm, 600 μm, 650 μm, 700 μm, 750 μm, 800 μm, 850 μm, 900 μm, 950 μm, or 1000 μm when measured a 30 mm or 60 mm distance from actuation.

48. The solution of claim 42, wherein the NORS provides a droplet size distribution of about 0.5 to about 2.0, wherein the droplet size distribution is: (Dv(90)−Dv(10))/Dv(50).

49. The solution of claim 42, wherein the at least one nitric oxide releasing compound is selected from the group consisting of a nitrite, a salt thereof, and any combinations thereof.

50. The solution of claim 42, wherein the at least one acidifying agent is an acid.

51-222. (canceled)
